(19) United States
(12) Patent Application Publication
(10) Pub. No.: US 2009/0068158 A1
(43) Pub. Date: Mar. 12, 2009

(54) THYMIDYLATE KINASE MUTANTS AND USES THEREOF

(76) Inventors: Jeffrey A. Medin, Toronto (CA); Arnon Lavie, Chicago, IL (US); Daniel H. Fowler, Bethesda, MD (US); Boro Dropulic, Ellicott City, MD (US)

Correspondence Address: FOX ROTHSCHILD LLP, 2000 MARKET STREET, 10th Floor, PHILADELPHIA, PA, 19103, US

(21) Appl. No.: 12/052,565

(22) Filed: Mar. 20, 2008

Related U.S. Application Data
(63) Continuation-in-part of application No. 11/559,757, filed on Nov. 14, 2006.
(60) Provisional application No. 60/748,828, filed on Dec. 9, 2005.

(51) Int. Cl. A61K 35/12; A61K 31/7088; C12N 15/87; C12N 5/10; C12N 15/63

(52) U.S. Cl. 424/93.21; 514/44; 435/455; 435/320.1; 435/325

(57) ABSTRACT

The invention relates to a composition comprising: a stably integrating delivery vector; a modified mammalian thymidylate kinase (tmpk) wherein the modified mammalian tmpk increases phosphorylation of a prodrug relative to phosphorylation of the prodrug by wild-type human tmpk. The invention also relates to use of these compositions in methods of treatment of diseaseuuius such as graft versus host disease and cancer.

### CROSS REFERENCE TO RELATED APPLICATION

This application is a continuation in part of U.S. patent application Ser. No. 11/559,757 filed Nov. 14, 2006 which claims priority from U.S. patent application No. 60/748,828, filed Dec. 9, 2005, the disclosures of which are incorporated herein by reference.

### FIELD OF THE INVENTION

The invention relates to compositions comprising a vector and modified thymidylate kinase polynucleotides. The compositions are useful in treatment of diseases such as cancer and graft versus host disease (GVHD).

### BACKGROUND OF THE INVENTION

Integrating viral vectors are a good choice for gene therapy because they offer fairly efficient transduction and consistent long-term gene expression. Much research has been directed towards improving vector design to increase safety and reliability. A promising approach is to establish control over the fate of transduced cells in vivo. Incorporating an effective suicide gene into a therapeutic vector can ensure that any malignant clones arising from deleterious insertion of the vector can be specifically killed. Likewise, such a control schema could be used as an inserted safety component for a variety of stem cell transplantations, reducing teratomas, for example, should these outgrowth events develop as occurred in one very recent study². A suicide gene schema can also be used to control post-transplant complications.

The infusion of donor lymphocytes in allogenic bone marrow transplant (BMT) recipients provides potent antitumor activity to treat recurrent malignancies. One complication, however, is severe GVHD (Graft Versus Host Disease), which is mediated by T cells in the graft. One approach to control GVHD is to employ suicide gene therapy.

Donor T cells mediate both GVHD and a GVL (Graft Versus Leukemia)-effect after allogenic haematopoietic stem cell transplantation (HCT), and the separation of GVL from GVHD has proven to be a formidable problem. The expression of an inducible suicide gene in donor T cells was conceived as a potential way to provide for the abrogation of GVHD after leukemic cells were eradicated. The most extensively studied suicide genes are derived from pathogens and include the HSV-tk and bacterial cytosine-deaminase genes, which encode enzymes that metabolize ganciclovir and 5-FU, respectively, and generate toxic active compounds (Carolina Berger, Mary E. Flowers, Edus H. Warren, Stanley R. Riddel. Analysis of transgene-specific immune responses that limit the in vivo persistence of adoptively transferred HSV-TK-modified donor T cells after allogenic hematopoietic cell transplantation. Blood 2006, 107:2294-302.)

In the customary adaptation of this approach, the herpes simplex virus type 1 thymidine kinase (HSV1-tk) gene, combined with the antiviral prodrug ganciclovir (GCV), is used to control GvHD after introduction of this suicide gene into donor T lymphocytes. However, the efficiency of HSV1-tk is suboptimal and the issue of host immunogenicity against this heterologous effector gene product can hamper outcomes. In addition, prophylactic GCV is often used to control cytomegalovirus infection after BMT. This confounds the broad clinical implementation of this approach.

HSV1-tk mediated cell killing requires cellular proliferation for its cytotoxic effect. This limits the effectiveness of gene therapies employing tk to only dividing cells. Quiescent disease cells will escape destruction and may persist. Tumor cells have been shown to remain quiescent for long periods of time (Trends Cell Biol. 15(9):494-501, 2005).

### SUMMARY OF THE INVENTION

The inventors' novel suicide gene therapy strategy combines the use of human thymidylate monophosphate kinase (tmpk) in a lentiviral vector (LV) format and the prodrug Zidovudine (AZT). Since tmpk is endogenously expressed in human cells, immunogenic responses will be limited.

It is an object of the invention to provide a vector construct comprising:
(i) a stably integrating delivery vector;
(ii) a modified mammalian thymidylate kinase (tmpk) wherein the modified mammalian tmpk increases phosphorylation of a prodrug relative to phosophorylation of the prodrug by wild-type mammalian (eg. human) tmpk.

It is an object of the invention to provide a composition optionally comprising:
(i) a stably integrating delivery vector;
(ii) a modified mammalian thymidylate kinase (tmpk) wherein the modified mammalian tmpk increases phosphorylation of a prodrug relative to phosophorylation of the prodrug by wild-type mammalian (eg. human) tmpk.

Optionally, increased phosphorylation can be determined in a side by side phosphorlyation assay comparing modified mammalian tmpk to wild-type mammalian (eg. human) tmpk.

The invention also optionally relates to use of these compositions in methods of treatment of diseases such as graft versus host disease and cancer.

It is another object of the invention to optionally provide a method of killing mammalian cells expressing a modified mammalian thymidylate kinase polynucleotide comprising:
i) contacting the mammalian cells with a composition of the invention;
ii) isolating the cells; and
iii) contacting the cells with a prodrug, such as AZT.

Another embodiment of the invention relates to a method of killing mammalian cells expressing a modified mammalian thymidylate kinase polynucleotide, comprising:
i) contacting the mammalian cells with a composition of the invention;
ii) isolating the cells;
iii) transplanting the cells into a transplant recipient; and
iv) administering a prodrug to the transplant recipient wherein the prodrug kills the cells.

It is another object of the invention to optionally provide a method of killing mammalian cells expressing a modified mammalian thymidylate kinase polynucleotide comprising:
i) contacting mammalian cells with a composition of the invention to produce modified cells expressing a modified mammalian thymidylate kinase;
ii) isolating said modified cells; and
iii) contacting said modified cells with a prodrug, such as AZT.

Another embodiment of the invention relates to a method of killing mammalian cells expressing a modified mammalian thymidylate kinase polynucleotide, comprising:
i) contacting the mammalian cells with a composition of the invention to produce modified cells expressing a modified mammalian thymidylate kinase;
ii) isolating said modified cells;
iii) transplanting said modified cells into a transplant recipient; and
iv) administering a prodrug to the transplant recipient wherein the prodrug kills the modified cells.

In another embodiment, the invention relates to a method of transplanting cells into a subject comprising administering mammalian cells of the invention expressing modified mammalian tmpk (preferably human cells expressing modified tmpk) or other suitable polynucleotide described herein, to the subject.

The invention also relates to a method of treating a transplant recipient exhibiting symptoms of a transplant mediated disease comprising administering a prodrug to the transplant recipient. The modified tmpk activates a prodrug by phosphorylation and the activated drug kills the modified tmpk-transduced cells. The method optionally further comprises detecting the presence of the mammalian cells in said transplant recipient one or more times during treatment. Graft versus host disease is an example of a transplant-mediated disease.

The invention also optionally relates to a suicide gene safety system for killing a genetically modified cell, the system comprising a vector comprising a suicide gene, such as modified mammalian tmpk or other suitable polynucleotide described herein, capable of activating a prodrug and a polynucleotide of interest to be expressed in said genetically modified cell. A polynucleotide of interest optionally includes a therapeutic molecule. Therapeutic molecules optionally include a normal gene, toxic molecules, cell growth enhancing molecules, or anti-sense molecules. Examples of therapeutic molecules of interest are described in this application, for example therapeutic molecules for treating Fabry disease.

In one embodiment, the invention relates to a composition comprising:
(i) a stably integrating delivery vector;
(ii) a modified mammalian thymidylate kinase (tmpk) polynucleotide wherein the modified mammalian tmpk polynucleotide encodes a modified mammalian tmpk polypeptide that increases phosphorylation of a prodrug relative to phosophorylation of the prodrug by wild type mammalian tmpk polypeptide.

Optionally the modified mammalian tmpk polypeptide increases phosphorylation of a prodrug relative to phosophorylation of the prodrug by the wild type mammalian tmpk polypeptides identified by a sequence identifier number in this application. The tmpk polynucleotide optionally comprises a polynucleotide with at least 80% sequence identity to a modified tmpk polynucleotide of any one of SEQ ID NOS: 15, 21, and 22. The modified mammalian tmpk polynucleotide optionally comprises a modified human tmpk polynucleotide. The polynucleotide optionally comprises a human polynucleotide and the polypeptides optionally comprise human polypeptides. The modified mammalian tmpk optionally comprises a truncated mammalian tmpk. The modified mammalian tmpk polynucleotide optionally comprises a mammalian tmpk polynucleotide with a point mutation. The point mutation optionally comprises a mutation in a codon of the polynucleotide selected from the group consisting of a mutation that encodes a F to Y mutation at amino acid position 105 (SEQ ID NO: 21), a mutation that encodes a R to G point mutation at amino acid position 16 (SEQ ID NO: 22), and a mutation that encodes a R to A mutation at amino acid position 200 (SEQ ID NO: 16). The polynucleotide optionally further comprises all or part of the large lid or small lid domain of E. coli (SEQ ID NO: 17). It will be readily apparent that one could make 2 or 3 or more amino acid changes. For example F105Y (SEQ ID NO:21) can be combined with R200A (SEQ ID NO: 22). Other combinations are also contemplated. In addition, other mutations not herein disclosed may be combined with the aforementioned mutations. For example, other mutations are readily modeled and derived from the crystal structure of tmpk. Mutations are optionally designed that are inert relative to the active site of the enzyme.

The polynucleotide optionally further comprises all or part of the large lid or small lid domain of E. coli (SEQ ID NO: 17). It will be readily apparent that all or part of large lid or small lid domains from other species of bacteria as well as other organisms such as yeast are useful. Utility is readily established by determining if the large lid or small lid from other sources increases phosphorylation of a prodrug relative to phosophorylation of the prodrug by wild type mammalian tmpk polypeptide.

The modified mammalian tmpk optionally comprises one or more deletions. The modified mammalian tmpk polynucleotide optionally has been modified by substituting a portion of wild type tmpk polynucleotide sequence with an exogenous polynucleotide sequence. The substituted portion comprises all or part of a large lid or small lid domain, for example, from E. coli. The exogenous sequence optionally comprises all or part of a bacterial sequence, optionally all or part of a bacterial small lid or large lid domain sequence, optionally an E. coli sequence, optionally TPEVGLKRARARGEL (SEQ ID NO: 17). The small lid domain optionally comprises all or part of amino acids AFGH corresponding to positions 145-148 of human tmpk of SEQ ID NO: 1. The exogenous sequence optionally comprises all or part of a bacterial sequence, optionally all or part of a bacterial small lid sequence, optionally an E. coli sequence, optionally all or part of the amino acid sequence RARGEL corresponding to positions 151-156 of SEQ ID NO: 17. The composition optionally further comprises a detection cassette (eg. detection/transduced cell enrichment cassette). The detection cassette is optionally selected from the group consisting of CD19, truncated CD19, EGFP, CD25, LNGFR, truncated LNGFR, CD24, truncated CD34, EpoR, HSA and CD20. The detection cassette optionally includes a drug resistance polynucleotide selected from the group comprising neomycin resistance polynucleotide, Bsr, Hph, Pac, Sh ble, FHT, bleomycin resistance polynucleotide and ampicillin resistance polynucleotide. The integrating viral vector optionally comprises an IRES sequence operably linked to the detection polynucleotide. The integrating viral vector optionally comprises a promoter operably linked to the detection polynucleotide. The composition optionally further comprises a therapeutic polynucleotide cassette.

The delivery vector is in one embodiment, selected from the group comprising a retroviral vector, an adenoviral vector, an adeno-associated viral vector, spumaviral, a lentiviral vector and a plasmid or other vector, such as transposons, described in the application. The retroviral vector optionally comprises an oncoretroviral vector. The retroviral vector optionally comprises a lentiviral vector. The vector is optionally a lentiviral vector that has a pHR′ backbone and comprises 5′-Long terminal repeat (LTR), HIV signal sequence, HIV Psi signal 5′-splice site (SD), delta-GAG element, Rev Responsive Element (RRE), 3′-splice site (SA), Elongation factor (EF) 1-alpha promoter and 3′-Self inactivating LTR (SIN-LTR). Optionally, one makes vectors with the CMV promoter. The lentiviral vector optionally comprises a central polypurine tract (cPPT; SEQ ID NO: 18) and a woodchuck hepatitis virus post-transcriptional regulatory element (WPRE; SEQ ID NO: 19), optionally the polypurine tract comprises nucleotide nos. 2023 to 2140 and the woodchuck hepatitis virus post-transcriptional regulatory element comprises nucleotide nos. 5802 to 6393 of (SEQ ID NO: 13 or the corresponding nucleotide numbers in SEQ ID NO:14); in a variation, optionally the vector comprises sequences comprising at least 70% sequence identity to one of the foregoing sequences. The lentiviral vector optionally comprises the nucleotides corresponding to the vector backbone portions of SEQ ID NO:13 or SEQ ID NO:14. The lentiviral delivery vector optionally comprises pLTG690 The vector construct optionally comprises pHR′-cppt-EF-tmpk(R16GLL)-IREShCD19-W-SIN (SEQ ID NO: 13). The vector construct optionally comprises pHR′-cppt-EF-tmpk-(F105Y)-IREShCD19-W-SIN (SEQ ID NO: 14). In another embodiment, the vector construct comprises pLTG690(pHR.RRE-cPPT-EF1a-Tmpk-1-CD19-WC-mPkg).

The composition optionally further comprises an additional kinase wherein the additional kinase contributes to activation of the prodrug. The compositions of the invention are optionally combined with a carrier and form a pharmaceutical composition.

The invention also includes a method of expressing a modified mammalian tmpk polynucleotide in a mammalian cell comprising contacting the mammalian cell with a composition of the invention. The mammalian cell is optionally a tumor cell. The tumor cell is optionally contacted with the composition in vivo, for example, using a method selected from the group consisting of microinjection, in vivo electroporation and liposome based methods. The method optionally further comprises administering an effective amount of a prodrug to eradicate the tumor cell. The prodrug optionally comprises AZT. The cells are optionally contacted using a method selected from the group consisting of transfection, infection and electroporation. The method optionally further comprises isolating the cells. The mammalian cells are optionally selected from the group consisting of stem cells, hematopoietic cells, T cells and human cells. The mammalian cells are optionally isolated by contacting the cells with an antibody that binds to a detection cassette protein wherein the detection cassette protein is selected from the group consisting of CD19, truncated CD19, EGFP, CD25, LNGFR, truncated LNGFR, CD24, truncated CD34, EpoR, HSA and CD20. The method optionally further comprises a step wherein the isolated mammalian cells are transplanted into a mammal. The mammalian cells are optionally transplanted to mediate tumor regression.

Another aspect of the invention relates to a method of killing mammalian cells expressing a modified mammalian tmpk polynucleotide comprising:
contacting the mammalian cells with a composition of the invention;
isolating the cells; and
contacting the cells with an effective amount of a prodrug to kill the cells.

The mammalian cells optionally comprise human cells, such as stem cells or hematopoietic cells (eg. T-cells, such as a CTL cell). The prodrug optionally comprises a substrate that is phosphorylated by a thymidylate kinase polypeptide. The prodrug is optionally selected from the group consisting of thymidine analog, uracil analog, AZT, dT4 and 5-FU.

Another embodiment of the invention relates to a method of killing mammalian cells expressing a thymidylate kinase polynucleotide comprising:
(i) contacting the mammalian cells with a composition of the invention;
(ii) isolating the cells;
(iii) transplanting the isolated cells into a transplant recipient; and
(iv) administering an effective amount of a prodrug to the transplant recipient to kill the transplanted, isolated cells.

The mammalian cells optionally comprise human cells, such as stem cells or hematopoietic cells (eg. T-cells, such as a CTL cell). The prodrug optionally comprises a substrate that is phosphorylated by a thymidylate kinase polypeptide. The prodrug is optionally selected from the group consisting of thymidine analog, uracil analog, AZT, dT4 and 5-FU.

The mammalian cells expressing said thymidylate kinase polynucleotide are optionally isolated by contacting the cells with an antibody that binds to a detection cassette protein wherein the detection cassette protein is optionally selected from the group consisting of CD19, truncated CD19, EGFP, CD25, LNGFR, truncated LNGFR, CD24, truncated CD34, EpoR, HSA and CD20. The transplant recipient is typically a human and, in certain embodiments, the transplant recipient has, or exhibits, symptoms of graft versus host disease.

Another aspect relates to a method of expressing a modified mammalian tmpk polynucleotide in a subject comprising:
administering a composition described herein to the subject wherein administration of the composition results in expression of the modified tmpk polynucleotide.

Also provided is a method of killing a mammalian cell in a subject expressing a modified mammalian tmpk polynucleotide comprising:
administering a composition of described herein; and
administering an effective amount of a prodrug to kill the cell.

Another aspect of the invention relates to a suicide gene system comprising:
a stably integrating delivery vector;
a modified mammalian tmpk wherein the modified mammalian tmpk increases phosphorylation of a prodrug relative to phosophorylation of the prodrug by wild type human tmpk; and
a prodrug that is phosphorylated and activated by the modified mammalian tmpk.

Another aspect of the invention relates to a suicide gene vector comprising a modified mammalian tmpk; and a detection cassette. The vector optionally further comprises a therapeutic cassette. The therapeutic cassette is optionally under the control of a tissue specific promoter and/or an inducible promoter.

Another aspect of the invention relates to an actuable cell destruction component of an expression vector comprising:
a modified mammalian tmpk polynucleotide wherein the modified mammalian tmpk polynucleotide increases phosphorlyation of a prodrug relative to phosphorylation of the prodrug by wild type mammalian tmpk;
a therapeutic polynucleotide for expression.

In the actuable cell destruction component, the therapeutic polynucleotide is optionally selected from the group comprising: adenosine deaminase, γc interleukin receptor subunit, α-galactosidase A, acid ceramidase, galactocerebrosidase, and CFTR molecules.

Another aspect of the invention relates to a method of killing a cell expressing a modified tmpk polynucleotide comprising contacting the cell with a prodrug that is activated by a composition of the invention. The prodrug is optionally a thymidine analog, such as AZT. The modified tmpk polynucleotide is optionally selected from the group comprising SEQ ID NO: 11, SEQ ID NO: 12, SEQ ID NO: 15; SEQ ID NO: 16, SEQ ID NO: 21 and SEQ ID NO: 22.

Another aspect of the invention relates to a method of killing a cell expressing a modified tmpk polynucleotide in a transplant recipient comprising administering an effective amount of a prodrug that is activated by the modified tmpk polynucleotide. The prodrug is optionally a thymidine analog, such as AZT. In one embodiment, the transplant recipient developed a transplant related adverse event, such as graft versus host disease.

Another aspect of the invention relates to a method of reducing cell proliferation, such as treating cancer, in a mammal in need thereof comprising:
contacting a mammalian cell with a composition of the invention to produce modified cells expressing the modified mammalian tmpk;
isolating the modified cells; and
transplanting said modified cells in the mammal wherein the modified cells induce a graft versus cancer effect.

The method optionally further comprises determining if the transplanted cells induce symptoms of graft versus host disease in the transplant recipient. The method optionally further comprises administering an effective amount of a prodrug to a transplant recipient who exhibits symptoms of graft versus host disease. In a variation, the cancer is leukemia.

Another embodiment of the invention relates to a method of identifying novel thymidine and uracil analog compounds that are useful as prodrugs in combination with a modified tmpk molecule comprising determining if a thymidine or uracil analog is phosphorylated by the modified tmpk molecule. Optionally the determining step comprises, a cell based assay comprising the steps of:
i) introducing a modified tmpk molecule into a cell;
ii) providing a thymidine analog; and
iii) determining whether said thymidine analog is a substrate for said modified tmpk.

The determining step optionally comprises a cell free assay comprising the steps of:
i) providing an enzymatically active modified tmpk,
ii) providing a thymidine analog;
iii) determining whether said thymidine analog is a substrate for said modified tmpk.

The application also provides for use a composition described herein.

In one embodiment, the application provides use of a composition described herein for expressing a modified mammalian tmpk polynucleotide in a mammalian cell or subject. In another embodiment the application provides use of a composition described herein for gene therapy. A further embodiment provides use of a composition described herein for treating a disease selected from the group consisting of cancer, GVHD or diseases resulting from a deficient gene product. Yet a further embodiment provides use of an effective amount of a prodrug for killing a cell expressing a modified mammalian tmpk polynucleotide wherein the expression of the modified mammalian tmpk results from contact with a composition described herein.

Other features and advantages of the present invention will become apparent from the following detailed description. It should be understood, however, that the detailed description and the specific examples while indicating preferred embodiments of the invention are given by way of illustration only, since various changes and modifications within the scope of the invention will become apparent to those skilled in the art from this detailed description.

### BRIEF DESCRIPTION OF THE DRAWINGS

Preferred embodiments of the invention will be described in relation to the drawings in which:

FIG. 1 is a schematic diagram of recombinant lentiviral transfer vector constructs. A. pHR′-tmpk-IRES-hCD19 is a schematic diagram of a lentiviral vector used to express wild-type tmpk, and mutants F105Y, R-16G-large lid and R200A in combination with a truncated CD19 detection molecule. B. pHR′-IRES-hCD19 is a schematic diagram of a lentiviral vector used to express a truncated CD19 detection molecule. C. pHR′-EGFP is a schematic diagram of a lentiviral vector used to express an EGFP detection molecule. The vector elements illustrated are: LTR—long terminal repeat; ψ—HIV packaging signal, SD—5′ splice signal, RRE—Rev responsive element; SA—3′ splice site, cPPT—central polypurine tract, EF1α—elongation factor 1α promoter; WPRE woodchuck hepatitis virus post-transcriptional regulatory element; SIN—self-inactivating LTR.

FIG. 2 shows a Western blot analysis of tmpk-overexpression by LV-transduction in Jurkat cells. NT: Non-transduced Jurkat cells, IRES: LV-IRES-hCD19-transduced Jurkat cells, WT: LV-(tmpk wild-type)-IRES-hCD19-transduced Jurkat cells, LL: LV-tmpk (R16G, Large lid)-IRES-hCD19-transduced Jurkat cells, F105Y: LV-tmpk (F105Y)-IRES-hCD19-transduced Jurkat cells.

FIG. 3 is a series of graphs comparing transduction efficiencies and hCD19 expression levels in LV-transduced Jurkat cells. Percentages indicate EGFP or CD19 expression and mean fluorescence intensity (MFI) values indicate the levels of expression levels in the cells.

FIG. 4A is a graph illustrating the AZT-sensitivity of Jurkat cells (human T cell line) transduced with LV-tmpk-IRES-hCD19 and mutant tmpk forms. Cell viability was determined by MTT assay (Promega). **, P<0.01, n=3. Data are expressed as mean±standard error of mean (SEM).

FIG. 4B is a series of plots showing annexinV staining.

FIG. 5 is a graph illustrating the induction of apoptosis by the addition of 100 μM AZT in LV-tmpk-transduced Jurkat cells. Cells were seeded in 24 well plates (10⁶/well) in 1 ml of medium with or without 100 μM of AZT. The medium was changed daily. After 4 days of culture, induction of apoptosis in the cells was analyzed by annexin-V staining according to the manufacturer's protocol (Annexin V-APC: BD Pharmingen). **, P<0.01, n=3. Data are expressed as mean±SEM.

FIG. 6A is a graph showing the levels of AZT-metabolites in the cells treated with 100 μM AZT. The cells were cultured in the presence of 100 μM AZT for 36 hrs. 10⁷ cells were homogenized by sonication in 100 ml of 5% (w/v) trichloroacetic acid. The supernatant is collected after homogenate had been centrifuged at 10,000×g for 15 min at 4° C. The trichloroacetic acid was removed by extraction with an equal volume of 20% tri-n-octylamine in pentane. The neutralized aqueous fraction is directly injected into HPLC. Separation of AZT and its metabolites was performed on a C18 column (Waters, Milford Mass.) with a mobile phase composed of 0.2 M phosphate buffer containing 4 mM tetrabutylammonium hydrogen sulfate (pH 7.5) and acetonitrile in the ratio of 97:3 (v/v). The mobile phase was pumped at a flow rate of 1.5 ml/min. The UV absorbance was monitored at 270 nm. Five million cell equivalents were injected and analyzed in triplicate.

FIG. 6B Determination of AZT metabolites in transduced clonal Jurkat cell lines and controls treated with 100 μM AZT. (a) Representative chromatograms for the NT cells and the tmpk R16G-mutant expressing cells. Each arrow indicates the position of a peak of the standard for AZT-MP, AZT-DP, and AZT-TP, respectively. (b) Comparison of the ratio of the intracellular AZT-TP to AZT-MP in the AZT-treated cells. Data are mean±SEM, n=3. The statistical differences were evaluated by the one-way analysis of variance (ANOVA) followed by a Bonferroni post-hoc test with the level of significance set at P<0.05.

FIG. 7 is a graph showing that LV-tmpk-transduced Jurkat cells exhibit an increase in the loss of mitochondrial membrane potential following incubation of the cells with AZT. Cells (10⁶ cells) treated with (shown (+) in figure) or without (−) 100 μM AZT were stained with JC-1 for 15 min at 37° C., and then were analyzed by flow cytometry. ***, P<0.001, n=3.

FIG. 8 is a series of graphs showing that AZT can induce apoptosis in the absence of cellular proliferation. Cellular proliferation is not always a prerequisite for AZT-induced apoptosis. Cells were seeded in 24-well plates (10⁶/well) in 1 ml of medium containing 0 (shown in AZT (−) in figure) or 100 μM of AZT (shown in AZT (+)) with or without 5 μM indirubin-3′-oxime (Figure (B) and (A), respectively). The medium was refreshed daily. After 2 days of culture, induction of apoptosis by AZT was analyzed by annexin V staining according to the manufacturer's protocol described. **, P<0.01, n=3. Data are expressed as mean±SEM.

FIG. 9 is a graph showing that mutant forms of tmpk prevent growth of transduced K562 cells xenografted into AZT-treated NOD/SCID mice. Female or male 5 to 8-week-old non-obese diabetic/sever combined immunodeficient (NOD/SCID) mice were purchased from Jackson Laboratory. Lentivirally-transduced or non-transduced K562 cells (20×10⁶ cells) were resuspended in 0.5 mL Dulbecco's phosphate-buffered saline (D-PBS) per inoculum and injected subcutaneously (SC) into the right flanks of recipient mice. AZT treatment, which was administered intraperitoneally (IP) at the dose of 2.5 mg/kg/day, was started one day after injection and conducted for 14 days. In vivo tumor cell growth was monitored by measuring tumor size for up to 32 days post-inoculations. All experimental data were reproduced at least twice.

FIG. 10 is a series of graphs evaluating the transduction efficiencies in the infected primary human T cells by detecting the transgene expression 6 days after transduction. 1-3: Transgene expression in primary human T cells transduced with 1:LV-EGFP, 2: LV-(tmpk R-16GLL)-IRES-hCD19, 3: LV-IRES-hCD19 Condition A-No transduction, Condition B-Single transduction using fibronectin (FN), Condition C-Three repeated transductions using FN, Condition D-Single transduction without FN, Condition E-Three transductions without FN. The cells are transduced repeatedly every 24 h at the MOI indicated.

FIG. 11 is a graph confirming transgene expression in the primary cultured mouse T cells isolated from spleen. Primary murine splenic T cells were isolated from the spleen of a Balb/c mouse. The cells were cultured for 3 days using anti-CD3/28 beads and 20 IU/ml recombinant human interleukin-2 (rhIL2). Cells were transduced using fibronectin (FN)-coated plates using an MOI of 20. EGFP-expression in the infected cells was confirmed 6 days post-transduction. Data are expressed as mean±SEM. P<0.001, n=3.

FIG. 12 is a graph comparing transgene expression in the cultured primary mouse T cells isolated from the spleen. The activated murine T cells were transduced with LVs indicated in the figure using either an FN-coated plate or transduction-on-ice methods. Transgene expression in the infected cells was confirmed 6 days post-transduction, n=2.

FIG. 13 is a listing of sequences.

FIG. 14 is a graph showing the measurement of AZT sensitivity of clonally-derived Jurkat cells transduced with LV-tmpk-IRES-huCD19Δ and control vectors. Cell viability was measured by MTT assay following 4 days incubation with or without AZT. The results were shown as percentage of the A595 nm value from the assay. The negative control values (without AZT) and the values without cells were deemed as 100% and 0%, respectively. Data are presented as the mean±SEM, n=3. The statistical significance of experimental observation was determined by the one-way ANOVA followed by a Dunnett post-hoc test with the level of significance set at P<0.05 compared with the values of the control group of cells that were not treated with AZT. *, P<0.05, and **, P<0.01 vs. the cells without AZT-treatment in each group.

FIG. 15 shows the induction of apoptosis by addition of 100 μM AZT in clonal Jurkat cells transduced with LV-tmpk-IRES-huCD19Δ and control vectors. Cells were cultured in the absence (A) or presence (B) of 5 μM indirubin-3′-monoxime for 4 days with or without 100 μM AZT. To compare the effect of AZT on induction of apoptosis in each group, measurement of flow cytometric analyses obtained from the cells treated with AZT were normalized by dividing values by those obtained without AZT. Data are mean±SEM, n=3. The statistical differences were evaluated by the one-way ANOVA followed by a Bonferroni post-hoc test with the level of significance set at P<0.05. **, P<0.01 vs. the NT cells.

FIG. 16 shows the transduction of primary murine and human T cells. (A) Observed levels of huCD19Δ expression on primary murine T cells 5 days after cells were transduced a single time with concentrated LV at an MOI of 20. (B) Observed levels of huCD19Δ expression on transduced primary human T cells. (C) Fold increases in the apoptotic index in the presence of 100 μM AZT.

FIG. 17 presents an analysis of the mechanism of induction of apoptosis by AZT in the tmpk-mutant expressing cells. (A) The tmpk mutant expressing cells treated with AZT showed an increase in the loss of mitochondrial membrane potential. Following 4 days incubation with or without 100 μM AZT, cells were stained with JC-1 for 15 min at 37° C., and then were analyzed by flow cytometry. To compare the effect of AZT on the increase in the loss of mitochondrial membrane potential at the day 4, the statistical differences were evaluated by the one-way ANOVA followed by a Bonferroni post-test with the level of significance set at P<0.05. ***, P<0.001, n=3. (B) Activation of caspase 3 in transduced cells by AZT treatment. Cells were cultured for 4 days with or without 100 μM AZT. To compare the effect of AZT on activation of caspase 3 in each group, measurement of flow cytometric analysis obtained from the cells treated with AZT were normalized by dividing those without AZT. Data are mean±SEM, n=3. The statistical differences were evaluated by the one-way ANOVA followed by a Bonferroni post-test with the level of significance set at P<0.05. **, P<0.01 and ***, P<0.001 vs. NT.

FIG. 18 shows that a daily injection of AZT prevents growth of K562 cells transduced with LV-tmpk-mutant in NOD/SCID mice. (A) NOD/SCID mice were subcutaneously injected with 2×10⁷ cells of either the NT or the LV-transduced K562 cells into the dorsal right flank. Starting one day after the cell injection, the mice received daily intraperitoneal injections of AZT (2.5 mg/kg/day) for two weeks. Tumor volume was monitored at the day indicated in the figure. (B) The tumor volume on day 14 (at the end point of the experiment) is shown. Data are mean±SD, n=5. The statistical comparison of means was performed by a two-tailed unpaired Student's t test.

FIG. 19 is a plasmid map of pLG690 comprising tmpk-IRES-truncated CD19.

FIG. 20 provides the sequence of a tmpk IRES truncated CD19.

FIG. 21 is a graph showing the AZT sensitivity in LV-TMPK transduced Jurkat cells.

FIG. 22 is a graph showing the AZT sensitivity in LV-TMPK transduced primary human T cells.

FIG. 23 is a FACS analysis of LV-TMPK transduced HEK 293 cells.

### DETAILED DESCRIPTION OF THE INVENTION

The inventors herein present a novel prodrug/enzyme combination for suicide gene therapy. Catalytically improved variants of human tmpk were delivered into target cells by novel lentiviruses (LVs), and the ability to selectively clear these cells in vitro and in vivo in response to increasing AZT concentrations was thoroughly evaluated. The inventors demonstrate the highly efficient transfer of these suicide genes and truncated huCD19 marker into murine and human T cells and cell lines. AZT sensitivity in transduced cells was further analysed. The inventors additionally demonstrate that increased accumulation of intracellular AZT-TP in tmpk-mutant-transduced cells decreases cell viability and that this is in part due to the activation of a mitochondria-mediated apoptosis pathway. These results show that the rationally designed minimal mutants of tmpk employed are a practical choice for suicide gene therapy and establish the next generation of safer integrating viral vectors. In addition, this system is useful to endow stem cells (both embryonic and of later ontogeny) destined for utility in clinical transplantation, for example, with a reliable safety system.

Accordingly, the invention relates to methods of using tmpk gene mutants inserted in transplant cells for treatment of cancer and controlling transplant-associated graft versus host disease. A lentrivirus is optionally used to deliver tmpk. Other methods of delivery are also useful.

The invention works by increasing phosphorylation of prodrugs such as AZT. For example, the prodrug AZT is converted through a series of phosphorylation steps into AZT-triphosphate (AZT-TP)¹². This is the active metabolite that inhibits replication of the human immunodeficiency virus (HIV)¹³⁻¹⁵, and to a lesser extent, DNA replication in eukaryotic cells¹⁶. Safety profiles for this compound are well known and concentrations of AZT in the bloodstream of AIDS patients being treated with this agent can reach high levels. The rate-limiting step in the conversion of AZT to the toxic AZT-TP form is the intermediate step of phosphorylation of AZT-monophosphate (AZT-MP) to AZT-diphosphate (AZT-DP) catalyzed by the cellular thymidylate kinase (tmpk), which has a low enzymatic efficiency for AZT-MP¹⁷. Accumulation of AZT-metabolites in the cells of AZT-treated AIDS patients reportedly induces toxic mitochondrial myopathy¹⁸⁻²². To harness this dual toxicity of AZT-TP, the inventors developed a novel suicide gene therapy approach based on the engineered overexpression of human tmpk. In order to improve the processing of AZT-MP to AZT-DP, thereby increasing intracellular AZT-TP concentrations, the inventors have engineered minimally modified tmpk mutants (F105Y+R200A and R16G-Large lid (RG16GLL)) with approximately 200-fold enhanced activity for AZT-MP^{23,24}.

Phosphorylation of the prodrug leads to its activation and increases its effectiveness in killing vector transduced cells (also called “suicide gene therapy”). The invention is useful in the event of a transplant related adverse event. A transplant related adverse event typically comprises graft versus host disease where following T-cell (or other cell) transplant to a recipient the transplanted cells attack the host. A transplant adverse event also comprises any situation where it would be beneficial to eliminate the transplanted cells, including where transplanted cells comprise integrations that can cause disease. The transplanted cells express mutant tmpk so that upon detection of graft versus host disease, a prodrug such as AZT is optionally administered to the patient to kill the transplanted cells.

For cancer treatment, the above method is useful to treat leukemia where donor transplant cells are used to kill leukemic cells. The transplanted cells expressing tmpk are likely to also attack the host, so the invention allows the transplanted cells to be killed after detection of the onset of graft versus host disease.

In a variation of the invention, tmpk vectors are inserted directly into the solid tumor and expression of tmpk sensitizes the cells to the prodrug.

Additionally, the tmpk gene mutants are useful as a general ‘safety component’ in gene therapy. For example in patients with Severe Combined Immunodeficiency Disease (SCID), gene therapy has been used successfully to introduce deficient genes however at least one clinical trial was halted due to safety concerns arising from inappropriate DNA integrations. The prior art also includes much discussion about the dangers of gene therapy due to vector integrations that can cause cancer. The safety component overcomes this problem by allowing the transplanted cells to be destroyed upon administration of a prodrug.

### Tmpk Variants

Thymidylate kinase is a kinase that catalyzes the addition of a phosphoryl group to thymidylate as well as thymidine analogs such as AZT. Several wild-type human sequences have been reported. SEQ ID NOS: 1, 3, 5 and 7 are reported nucleotide sequences of human thymidylate kinase (SEQ ID NO: 7 does not have a stop codon). The different sequences represent natural polymorphic variations present in the population and it will be recognized in the art that future identified molecules with polymorphic variantions will also be considered to be wildtype tmpk. SEQ ID NO: 9 is the reported mouse thymidylate kinase sequence. The mouse sequence shares 82% nucleotide identity 81% amino acid identity and several residues that have been identified as limiting the nucleoside analog activity of the human tmpk enzyme and which result in increased enzymatic activity when modified, are conserved in the murine sequence. The corresponding amino acid sequences are reported in SEQ ID NOS: 2, 4, 6, 8, and 10. SEQ ID NO: 2 provides the amino acid sequence for the wild-type tmpk polynucleotide described in SEQ ID NO: 1; SEQ ID NO: 4 provides the amino acid sequence for the wild-type tmpk polynucleotide reported in SEQ ID NO: 3, SEQ ID NO: 6 provides the amino acid sequence for the wild-type tmpk polynucleotide described in SEQ ID NO: 5; SEQ ID NO: 8 provides the putative sequence of the wild-type tmpk polynucleotide reported in SEQ ID NO: 7; and SEQ ID NO: 10 provides the amino acid sequence of the wild-type murine tmpk polynucleotide described in SEQ ID NO: 9. Modified tmpk molecules and mutant tmpk refer to mammalian tmpk molecules that have been modified compared to wild-type. Among the mutant tmpks, some of these showed a superior enzymatic activity to convert deoxy-thymidine-monophosphate (dTMP) to dTMP-diphosphate (dTDP) or AZT-MP to AZT-DP. Increased kinase activity relative to wild-type refers to modified tmpk molecules that exhibit improved enzymatic kinetics compared to tmpk wild-type. The improved activity comprises increases in binding and or enzymatic turnover to convert the monophosphate-form of the substrate of tmpk to the diphosphate form.

Mutations which showed superior enzymatic activity included the F105Y mutant (SEQ ID NO: 11, SEQ ID NO: 21), R16GLL mutant (SEQ ID NO: 12, SEQ ID NO: 22) and the R200A mutant (SEQ ID NOS: 15 and 16). Combinations of mutations also showed enhanced enzymatic activity. For example, the combination of mutations F105Y+R200A also showed superior enzymatic activity.

One aspect of the invention provides delivery vectors comprising modified tmpk enzymes with increased nucleoside analog kinase activity relative to wild-type. In one aspect, the modification that increases tmpk nucleoside analog kinase activity comprises one or more deletions. The deletions can be internal or can result in a truncated variant. In an alternate embodiment the modification that increases tmpk nucleoside analog kinase activity comprises one or more point mutations. In another embodiment an exogenous sequence replaces an endogenous sequence. For example, in one embodiment all or part of the large lid domain of human tmpk (SEQ ID NO:20) is replaced with all or part of the large lid domain of a different species. In one embodiment the different species is a bacteria species. In one embodiment, all or part of the large lid domain of human tmpk (SEQ ID NO:20) is replaced with all or part of the large lid domain of E. coli tmpk (SEQ ID NO:17). In another embodiment, residues 145-148 of SEQ ID NO: 1 (AFGH) are replaced with all or part of the small lid region of E. coli residues 151-156 in SEQ ID NO: 17 (RARGEL). In another embodiment the modified tmpk is selected from the group including the F105Y mutant (SEQ ID NO: 11, SEQ ID NO: 21), R16GLL mutant (SEQ ID NO: 12, SEQ ID NO: 22), a tmpk molecule modified by the substitution of all or part of a bacterial large lid domain such as the E. coli large lid domain in SEQ ID NO: 17, a tmpk molecule modified by the substitution of all or part of a bacterial small lid domain such as the E. coli small lid domain at 151-156 of SEQ ID NO: 17, the R200A mutant (SEQ ID NOS: 15 and 16) and combinations thereof.

In another embodiment, the exogenous sequence is optionally synthesized or obtained from a non-mammalian thymidylate kinase such as a bacterial thymidylate kinase. As used herein a modified mammalian tmpk molecule includes a modified tmpk molecule that comprises non-mammalian sequences such as all or part of either a large lid domain or a small lid domain sequence from bacteria such as E. coli. A variant may comprise one or more of the aforementioned modifications. Examples of modifications are described above.

### Delivery Vectors

It will be appreciated by one skilled in the art that a variety of delivery vectors and expression vehicles are usefully employed to introduce a modified tmpk molecule into a cell. Vectors that are useful comprise lentiviruses, oncoretroviruses, expression plasmids, adenovirus, and adeno-associated virus. Other delivery vectors that are useful comprise herpes simplex viruses, transposons, vaccinia viruses, human papilloma virus, Simian immunodeficiency viruses, HTLV, human foamy virus and variants thereof. Further vectors that are useful comprise spumaviruses, mammalian type B retroviruses, mammalian type C retroviruses, avian type C retroviruses, mammalian type D retroviruses, HTLV/BLV type retroviruses, and lentiviruses.

Vectors such as those listed above have been employed to introduce thymidine kinase molecules into cells for use in gene therapy. Examples of vectors used to express thymidine kinase in cells include: Kanazawa T, Mizukami H, Okada T, Hanazono Y, Kume A, Nishino H, Takeuchi K, Kitamura K, Ichimura K, Ozawa K. Suicide gene therapy using AAV-HSVtk/ganciclovir in combination with irradiation results in regression of human head and neck cancer xenografts in nude mice. Gene Ther. 2003 January; 10(1):51-8. Fukui T, Hayashi Y, Kagami H, Yamamoto N, Fukuhara H, Tohnai I, Ueda M, Mizuno M, Yoshida J Suicide gene therapy for human oral squamous cell carcinoma cell lines with adeno-associated virus vector. Oral Oncol. 2001 April; 37(3):211-5.

### Lentirviral Vectors

The safety facet of suicide gene therapy relies on efficient delivery and stable, consistent expression of both the therapeutic and the cytotoxic effector genes. LVs transduce a wide range of dividing and non-dividing cell types with high efficiency, conferring stable, long-term expression of the transgene²⁵⁻²⁷.

The use of lentivirus-based gene transfer techniques relies on the in vitro production of recombinant lentiviral particles carrying a highly deleted viral genome in which the transgene of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, incapsidation, and expression, in which the sequences to be expressed are inserted.

In one embodiment the Lentigen lentiviral vector described in Lu, X. et al. Journal of gene medicine (2004) 6:963-973 is used to express the modified tmpk molecules.

In certain embodiments the invention comprises a lentiviral vector expressing a modified tmpk molecule. In one embodiment the lentiviral vector comprises a 5′-Long terminal repeat (LTR), HIV signal sequence, HIV Psi signal 5′-splice site (SD), delta-GAG element, Rev Responsive Element (RRE), 3′-splice site (SA), Elongation factor (EF) 1-alpha promoter and 3′-Self inactivating LTR (SIN-LTR). It will be readily apparent to one skilled in the art that optionally one or more of these regions is substituted with another region performing a similar function.

Gene therapy requires the transgene product to be expressed at sufficiently high levels. Enhancer elements can be used to increase expression of modified tmpk molecules or increase the lentiviral integration efficiency. In one embodiment the lentiviral vector further comprises a nef sequence. In certain embodiment the lentiviral further comprises a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. In an alternate embodiment, the lentiviral vector further comprises a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to lentiviral vector results in a substantial improvement in the level of transgene expression from several different promoters, both in vitro and in vivo. In a further embodiment, the lentiviral vector comprises both a cPPT sequence and WPRE sequence. The vector also comprises in an alternate embodiment an internal ribosome entry site (IRES) sequence that permits the expression of multiple polypeptides from a single promoter. In another embodiment the lentiviral vector comprises a detection cassette. In another embodiment, the detection cassette comprises a CD19 molecule or fragment thereof. In another embodiment the plasmid is pHR′-cppt-EF-IRES-W-SIN. SEQ ID NO: 13 provides the nucleotide sequence of pHR′-cppt-EF-tmpk(R16GLL)-IRES-hCD19-W-SIN. SEQ ID NO: 14 provides the nucleotide sequence of pHR′-cppt-EF-tmpk(F105Y)-IRES-hCD19-W-SIN. Additionally it will be readily apparent to one skilled in the art that optionally one or more of these elements can be added or substituted with other regions performing similar functions.

In addition to IRES sequences, other elements which permit expression of multiple polypeptides are useful. In one embodiment the vector comprises multiple promoters that permit expression more than one polypeptide. In another embodiment the vector comprises a protein cleavage site that allows expression of more than one polypeptide. Examples of protein cleavage sites that allow expression of more than one polypeptide comprise those listed in the following articles which are incorporated by reference: Retroviral vector-mediated expression of HoxB4 in hematopoietic cells using a novel coexpression strategy. Klump H, Schiedlmeier B, Vogt B, Ryan M, Ostertag W, Baum C. Gene Ther. 200; 8(10):811-7; A picornaviral 2A-like sequence-based tricistronic vector allowing for high-level therapeutic gene expression coupled to a dual-reporter system Mark J. Osborn, Angela Panoskaltsis-Mortari, Ron T. McElmurry, Scott K. Bell, Dario A. A. Vignali, Martin D. Ryan, Andrew C. Wilber, R. Scott Mclvor, Jakub Tolar and Bruce R. Blazar. Molecular Therapy 2005; 12 (3), 569-574; Development of 2A peptide-based strategies in the design of multicistronic vectors. Szymczak A L, Vignali D A. Expert Opin Biol Ther. 2005; 5(5):627-38; Correction of multi-gene deficiency in vivo using a single ‘self-cleaving’ 2A peptide-based retroviral vector. Szymczak A L, Workman C J, Wang Y, Vignali K M, Dilioglou S, Vanin E F, Vignali D A. Nat Biotechnol. 2004; 22(5):589-94. It will be readily apparent to one skilled in the art that other elements that permit expression of multiple polypeptides which identified in the future are useful and may be utilized in the vectors of the invention.

### Detection/Selection Cassettes

In suicide gene therapy, it is typically desirable that the majority of transduced cells express the suicide gene. This need can be met by co-introducing a cell surface marker gene. Transduced cells can be identified and enriched based on expression of this marker. A good cell surface marker should be inert in itself, devoid of signaling capacity and non-immunogenic²⁸. A variety of cell surface markers can be used in this context: human CD24²⁹, murine HSA³⁰, human CD25 (huCD25)³¹ and a truncated form of LNGFR³².

While huCD25 has been an efficient and malleable marker for murine studies^{27,33}, it is not useful for gene transfer applications into T cells since expression of this molecule is up-regulated when this population is activated. Other groups have also used the truncated form of LNGFR³², but overexpression of this marker has been reported to promote transformation of myeloid cells in an unusual, highly context-dependent manner³⁵. A novel truncated form of CD19 (CD19Δ) is optionally adopted as a marker (SEQ ID NOS: 29-31). CD19 (SEQ ID NOS: 27-28) is a 95-kDa glycoprotein of the immunoglobulin superfamily. It forms a complex with CD21, CD81, and Leu-13, and collectively functions to modulate the activation threshold of the B cell receptor^{36,37}. As expression of CD19 and CD21 is restricted to B cell lineages from immature progenitors to blasts³⁸, it is suitable for use in murine and human T cells. To further decrease any signaling capacity from the CD19 molecule, the cytoplasmic tail³⁹ has been deleted for the present adaptation. In one embodiment truncated CD19 comprises all or a portion of SEQ ID NO: 29. In another embodiment truncated CD19 comprises all or a portion of SEQ ID NO: 30. In another embodiment truncated CD19 comprises all or a portion of SEQ ID NO: 31.

“Detection cassette” is used to refer to a polynucleotide that directs expression of a molecule that acts as a cell marker and that optionally provides for a mode of isolating cells expressing said marker. The molecule is optionally used to select transduced or transfected cells or to determine the efficiency of cell transduction or transfection. Molecules that are useful as cell markers or detection agents comprise CD19, truncated CD19, CD25 and EGFP. EGFP is variably referred to as enGFP herein. One skilled in the art will recognize that other fluorescent molecules can similarly be used.

As mentioned, the detection cassette encodes a molecule that is typically used to isolate transduced or transfected cells. The detection cassette is useful in vectors comprising modified tmpk or control molecules. Control molecules include molecules that do not function as suicide gene therapy molecules which that are typically employed to assess the effect of tmpk mutants in similarly related cells. A person skilled in the art would recognize that many molecules are useful to permit isolation of modified tmpk or control expressing cells. Choice of molecule will depend on the cell type to be transfected or transduced. The detection cassette molecule is not expressed on the cell type to be transfected or transduced in appreciable levels permitting isolation of cells expressing the detection cassette. In one embodiment the detection cassette encodes a CD19 (SEQ ID NOS: 27-28). In one embodiment, the detection cassette encodes a truncated CD19 (SEQ ID NOS: 29-31). In an alternate embodiment, the detection cassette encodes CD25. In another embodiment, the detection cassette encodes a fluorescent protein such as EGFP. In another embodiment, the molecules encoded by the detection cassette comprise CD20, CD25, low affinity nerve growth factor receptor (LNGFR), truncated CD34, or erythropoietin receptor (EpoR). Additionally, the detection cassette can comprise a drug resistance gene permitting isolation of transduced or transfected cells by drug selection.

### Vector Constructs

One aspect of the invention provides a vector construct comprising:
(i) a stably integrating delivery vector;
(ii) a modified mammalian thymidylate kinase (tmpk) wherein the modified mammalian tmpk increases phosphorylation of a prodrug relative to phosophorylation of the prodrug by wild-type mammalian (eg. human) tmpk.

In certain embodiments, the vector construct further comprises a detection cassette. In other embodiments the vector construct comprises a therapeutic gene cassette. In still other embodiments, the vector construct further comprises both a detection cassette and a therapeutic gene. Other elements and components described herein can also be comprised in the vector construct.

In one embodiment the vector construct comprises a lentiviral delivery vector. In certain embodiments the lentiviral delivery vector is a Lentigen delivery vector described herein. In another embodiment the Lentigen delivery vector is pLTG690. In another embodiment the vector construct comprises Lentigen TMPK-CD19 vector. In a further embodiment the vector construct comprises pLTG690 (pHR.RRE-cPPT-EFla-Tmpk-1-CD19-WC-mPkg).

### Methods of Isolation

In one aspect of the present invention, methods for expressing a modified tmpk molecule in cells for transplant are provided. After transduction or transfection with vectors comprising a detection cassette and modified tmpk molecules or control molecules, cells expressing these molecules are optionally isolated by a variety of means known in the art. A molecule encoded by the detection cassette is used to isolate modified tmpk positive cells. In certain embodiments, the cells are isolated by cell sorting or flow cytometry using an antibody to the detection cassette encoded molecule. Additionally cell sorting is useful to isolate modified tmpk expressing cells where the detection cassette is a fluorescent protein such as EGFP. Cells expressing modified tmpk or control molecules are, in an alternate embodiment, isolated using magnetic sorting. Additionally, cells may be isolated by drug selection. In one embodiment, a vector comprising a drug resistance gene and a modified tmpk molecule is introduced into cells. Examples of drug resistance genes include, but are not limited to, neomycin resistance gene, blasticidin resistance gene (Bsr), hygromycin resistance gene (Hph), puromycin resistance gene (Pac), Zeocin resistance gene (Sh ble), FHT, bleomycin resistance gene and ampicillin resistance gene After transduction or transfection, cells expressing modified tmpk or control molecules and the drug resistance gene are selected by adding the drug that is inactivated by the drug resistance gene. Cells expressing the drug resistance gene survive while non-transfected or non-transduced cells are killed. A person skilled in the art would be familiar with the methods and reagents required to isolate cells expressing modified tmpk molecules.

### Cell Types for Transplant

Modified tmpk molecules are usefully introduced into any cell type ex vivo where it is desirable to provide a mechanism for killing the modified tmpk expressing cells. Cell types that are useful in one embodiment of the present invention include, but are not limited to, stem cells (both embryonic and of later ontogeny), cord blood cells, and immune cells such as T cells, bone marrow cells and peripheral blood mononuclear cells. T-cells are optionally CD4 positive, CD8 positive or double positive. In addition, T cells are optionally mature T cells. In one embodiment T cells are transduced with modified tmpk molecules, isolated and transplanted in a host. In another embodiment the T cells are mature T cells. In an alternate embodiment stem cells are transduced, isolated and transplanted in a host.

Cell lines are optionally transduced. For example human T cell leukemia Jurkat T cells, human erythro-leukemic K562 cells, human prostate cell lines DU145 and PC3 cells are optionally transduced or transfected with modified tmpk molecules.

### Prodrugs

A prodrug refers to a pharmacological substance (drug) which is administered in an inactive form (or significantly less active form, eg at least 90% or at least 95% less active than the active drug form). Once administered, the prodrug is metabolised in the body (in vivo) into the active compound and these metabolites provide cytotoxicity against the cells.

A prodrug is useful in combination with suicide gene therapy strategies. Suicide genes that make transduced cells susceptible to a molecule that is not ordinarily toxic function as a safety mechanism. The most commonly used suicide gene is the thymidine kinase gene from herpes simplex type I virus (HSV1-tk).

AZT is an example of a nucleoside prodrug that is poorly phosphorylated by thymidylate kinase enzymes. Other thymidine and uracil analogs are known and would be useful as prodrugs for killing cells expressing modified tmpk. Other known thymidine and uracil analogues that are useful comprise d4T and 5-FU. Additional thymidine and uracil analogs are known in the art. (J Med Chem. 1996 39(17):3412-7 Synthesis and evaluation of novel thymidine analogs as antitumor and antiviral agents. Chen X, Bastow K, Goz B, Kucera L S, Morris-Natschke S L, Ishaq K S).

In another embodiment, the prodrug administered is AZT. In an alternate embodiment the prodrug is a thymidine analog that is a substrate for modified tmpk enzymes. In another embodiment the prodrug is a uracil analog.

Prodrugs may require more than one enzyme activation. For example ganciclovir requires phosphorylation by thymidine kinase as well as a second phosphorylation guanylate kinase. In one embodiment of the present invention, a method of tandem expression of modified tmpk and additional kinases required for prodrug is provided.

### Methods of Treatment

### Treatment of Cancer

The present invention provides methods and uses of modified tmpk molecules for the reduction of cell proliferation, for example for treatment of cancer. The present invention also provides methods of expressing modified tmpk molecules for the reduction of cell proliferation, for example for treatment of cancer.

Modified tmpk is introduced into cells that are used for transplant or introduced directly in vivo in mammals, preferably a human. The modified tmpk molecules are typically introduced into cells ex vivo using methods known in the art. Methods for introducing tmpk molecules comprise transfection, infection, electroporation. These methods optionally employ liposomes or liposome like compounds.

In one embodiment, modified tmpk molecules are used to treat cancer by adoptive therapy. Adoptive therapy or adoptive (immuno)therapy refers to the passive transfer of immunologically competent tumor-reactive cells into the tumor-bearing host to, directly or indirectly, mediate tumor regression. The feasibility of adoptive (immuno)therapy of cancer is based on two fundamental observations. The first of these observations is that tumor cells express unique antigens that can elicit an immune response within the syngeneic (genetically identical or similar especially with respect to antigens or immunological reactions) host. The other is that the immune rejection of established tumors can be mediated by the adoptive transfer of appropriately sensitized lymphoid cells. Clinical applications include transfer of peripheral blood stem cells following non-myeloablative chemotherapy with or without radiation in patients with lymphomas, leukemias, and solid tumors.

In one aspect of the present invention, donor T cells or stem cells (either embryonic or of later ontogeny) are transduced with vectors comprising modified tmpk molecules. Cells expressing said modified tmpk are isolated and adoptively transferred to a host in need of treatment. In one embodiment the bone marrow of the recipient is T-cell depleted. Methods of adoptive T-cell transfer are known in the art (J Translational Medicine, 2005 3(17): doi;0.1186/1479-5876-3-17, Adoptive T cell therapy: Addressing challenges in cancer immunotherapy. Cassian Yee). This method is used to treat solid tumors and does not require targeting the modified tmpk expressing T-cells to the tumor since the modified tmpk donor T-cells will recognize the different MHC class molecules present in the recipient host resulting in cytotoxic killing of tumor cells.

Another aspect of the invention provides for the treatment of solid tumors by injecting vectors carrying modified tmpk molecules directly into the tumor. Methods of introducing modified tmpk directly in vivo in a mammal, preferably a human, comprise direct viral delivery, microinjection, in vivo electroporation, and liposome mediated methods.

Thymidine kinase has been introduced by injection directly into the site of a tumor to examine results of the technique as a cancer therapeutic treatment (Chevez-Barrios P, Chintagumpala M, Mieler W, Paysse E, Boniuk M, Kozinetz C, Hurwitz M Y, Hurwitz R L. Response of retinoblastoma with vitreous tumor seeding to adenovirus-mediated delivery of thymidine kinase followed by ganciclovir. J Clin Oncol. 2005 Nov. 1; 23(31):7927-35. Sterman D H, Treat J, Litzky L A, Amin K M, Coonrod L, Molnar-Kimber K, Recio A, Knox L, Wilson J M, Albelda S M, Kaiser L R. Adenovirus-mediated herpes simplex virus thymidine kinase/ganciclovir gene therapy in patients with localized malignancy: results of a phase I clinical trial in malignant mesothelioma. Hum Gene Ther. 1998 May 1; 9(7):1083-92). The tmpk molecules of the present invention are optionally introduced directly into the site of a tumor to reduce proliferation of tumor cells, for example, to treat cancer.

In one embodiment, cells are transfected or transduced ex vivo with modified tmpk vectors. In an optional embodiment, the vector comprises a lentiviral vector.

### Tissue Specific Expression

In an alternate embodiment of the invention, the modified tmpk expressing cells express tmpk under the control of a tissue or cell specific promoter providing expression in a tissue specific manner. Expression of modified tmpk molecules is optionally targeted to tumor cells using promoters that are active in tumor cells.

Accordingly, in one aspect of the invention, delivery vectors comprising modified tmpk molecules are provided that result in tissue or cell specific expression of the modified tmpk molecules. Tissue and cell specific expression of modified tmpk is typically accomplished using promoters operably linked with the modified tmpk, which limit expression of modified tmpk to cells or tissues. One skilled in the art will recognize that a variety of promoter sequences that direct tissue or cell specific expression are useful to direct tissue or cell specific expression of modified tmpk. For example, one skilled in the art will readily recognize that liver specific expression is accomplished using a liver specific promoter. Modified tmpk expression is readily limited to a variety of cell and tissue types. Examples include, but are not limited to, liver, pancreas and T cells. Examples of liver specific promoters include, but are not limited to, the transthyretin promoter, albumin promoter, alpha feto protein promoter. Examples of other cell specific promoters include, but are not limited to, islet cell specific promoters such as the insulin promoter, and T cell specific promoters such as CD4-promoter. In another embodiment, expression of modified tmpk is inducible. The hypoxia-inducible promoter is optionally used to direct expression of a cytoprotective gene such as but not limited to erythropoietin. Introduction of a cytoprotective gene under the control of a inducible promoter such as the hypoxia inducible promoter is useful, to prevent the severe tissue damage by hypoxia.

If the transduced cells cause some problems, the transduced cells are optionally cleared (killed) by suicide effect by administering prodrug to the transduced cells.

Tumor cell specific expression is accomplished using a tumor specific promoter. Tumor specific promoters comprise the progression elevated gene-3 (PEG-3) promoter. This promoter functions selectively in divergence cancer cells with limited activity in normal cells, for tumor cell-specific expression. The transduced tumor cells are specifically killed by the prodrug.

### Graft Versus Leukemia

In addition, the invention provides, in one aspect, a method of treating leukemia. Donor T cells or stem cells are transduced with vectors comprising modified tmpk molecules, cells expressing said modified tmpk are isolated and transplanted to a host in need of treatment. The transplanted cells induce a graft versus leukemia effect. If the transplanted cells induce graft versus host disease, the transplanted cells can be killed by administering a prodrug.

Graft versus leukemia refers to using donor transplant cells to kill host leukemic cells. Introduced cells will often also attack the cancer cells that still may be present after transplant. This was first documented in acute leukemia, and this phenomenon has been called “graft-versus-leukemia” effect. Similar effects have been observed in malignant lymphoma, myeloma, and perhaps even some solid tumors. For certain diseases, such as chronic myelogenous leukemia (CML), the graft-versus-leukemia (GvL) effect may well be the most important reason that allogeneic transplants are successful in curing the disease.

### Graft Versus Host Disease (GVHD)

Graft versus host disease is a common complication of allogeneic bone marrow transplantation (BMT). After bone marrow transplantation, T cells present in the graft, either as contaminants or intentionally introduced into the host, attack the tissues of the transplant recipient. Graft-versus-host disease can occur even when HLA-identical siblings are the donors. HLA-identical siblings or HLA-identical unrelated donors (called a minor mismatch as opposed to differences in the HLA antigens, which constitute a major mismatch) often still have genetically different proteins that can be presented on the MHC.

Graft versus host disease is a serious complication of transplant and can lead to death in patients that develop severe graft versus host disease (the clinical manifestations of graft versus host disease are reviewed in Socie G. Chronic graft-versus-host disease: clinical features and grading systems. Int J Hematol. 2004 April; 79(3):216-20). Viral thymidine kinase has been introduced into transplant cells and used in combination with drugs such as ganciclovir to determine the results in individuals who develop graft versus host disease. (Bonini C, Ferrari G, Verzeletti S, Servida P, Zappone E, Ruggieri L, Ponzoni M, Rossini S, Mavilio F, Traversari C, Bordignon C HSV-TK gene transfer into donor lymphocytes for control of allogeneic graft-versus-leukemia. Science. 1997 Jun. 13; 276(5319):1719-24; Bondanza A, Valtolina V, Magnani Z, Ponzoni M, Fleischhauer K, Bonyhadi M, Traversari C, Sanvito F, Toma S, Radrizzani M, La Seta-Catamancio S, Ciceri F, Bordignon C, Bonini C Suicide gene therapy of graft-versus-host disease induced by central memory human T lymphocytes. Blood. 2005.)

While donor T-cells are undesirable as effector cells of graft-versus-host-disease, they are valuable for engraftment by preventing the recipient's residual immune system from rejecting the bone marrow graft (host-versus-graft). Additionally, as bone marrow transplantation is frequently used to cure malignant disorders (most prominently the leukemias), donor T-cells have proven to have a valuable graft-versus-tumor (GVT, graft versus leukemia described above) effect. A great deal of current research on allogeneic bone marrow transplantation involves attempts to separate the undesirable graft-vs-host-disease aspects of T-cell physiology from the desirable graft-versus-tumor effect.

The present invention provides, in one embodiment, methods of treating transplant patients that develop graft versus host disease by administering compounds of the invention (ie. tmpk mutants used in combination with drugs) to a mammal in need thereof. In another embodiment, the invention provides a method of promoting graft versus tumor effect by administering compounds of the invention to a mammal in need thereof.

### Safety Component for Gene Therapy

One problem with the use of gene therapy to stably introduce exogenous polynucleotides is the potential to develop a gene therapy related disease such as cancer. A gene therapy vector can integrate into a DNA region that could causes cancer in the gene therapy patient.

In one embodiment of the invention, tmpk mutants are useful as a safety component in gene therapy constructs. It is clear to one skilled in the art that the tmpk mutants are useful in combination with different polynucleotides designed to treat a variety of conditions. The tmpk mutants are useful in combination with a polynucleotide that encodes a polypeptide that compensates for a deficient gene product. Examples of diseases that comprise a deficient gene product include, but not are limited to, Factor IX deficiency, Factor VIII deficiency, Gaucher disease, SCID, MPS, cystic fibrosis, Fabry disease, Farber disease, sickle cell disease, chronic granulomatous disorder (CGD). In this aspect of the invention, vectors comprising a tmpk mutant and a deficient gene are introduced into cells ex vivo such as bone marrow cells or provided systemically to a patient deficient in the gene product. Systemically introduced vectors can integrate into host cells forming gene-modified cells. If the gene-modified cells cause a gene therapy related disease in the recipient model, a prodrug is administered to the recipient that kills the gene-modified cells.

### Drug Discovery Platform

The present invention also provides assays for identifying novel thymidine and uracil analog compounds that are useful as prodrugs in combination with modified tmpk molecules. The thymidine analogs can be synthesized according to methods known in the art (J Med Chem. 1996 39(17):3412-7 Synthesis and evaluation of novel thymidine analogs as antitumor and antiviral agents. Chen X, Bastow K, Goz B, Kucera L S, Morris-Natschke S L, Ishaq K S) and tested for the use as substrates for modified tmpk. Alternatively libraries of thymidine or uracil analogs can be synthesized and screened for compounds that can act as substrates for modified tmpk. Methods for the synthesis of molecular libraries are known in the art (Novel nucleotide analogues as potential substrates for TMPK, a key enzyme in the metabolism of AZT. Muller H C, Meier C, Balzarini J, Reinstein J. Nucleosides Nucleotides Nucleic Acids. 2003; 22(5-8):821-3).

In one aspect of the present invention, compounds are identified using rational drug design and tested for their use as substrates for modified tmpk.

In one embodiment the assay comprises, a cell-based assay comprising the steps of:
i) introducing a modified tmpk molecule into a cell;
ii) providing a thymidine analog to the cell; and
iii) determining whether said thymidine analog is a substrate for said modified tmpk.

In an alternate embodiment, the assay comprises a cell free assay comprising the steps of:
i) providing an enzymatically active modified tmpk,
ii) providing a thymidine analog to the modified tmpk;
iii) determining whether said thymidine analog is a substrate for said modified tmpk.

The enzymatically active modified tmpk can comprise a fusion protein such as a GST fusion protein. In one embodiment the assay is conducted in a test tube. In an alternative embodiment the assay is conducted in a micro-titer plate. The enzymatically active modified tmpk can be free in solution or bound to beads such as sepharose beads. The determination of whether said thymidine analog is a substrate for said modified tmpk can comprise the use radioactive phosphorus or non-radioactive means. The methods of assessing kinase activity and substrate utilization are well known in the art.

### Viral Regulatory Elements

The viral regulatory elements are components of vehicles used to introduce nucleic acid molecules into a host cell. The viral regulatory elements are optionally retroviral regulatory elements. For example, the viral regulatory elements may be the LTR and gag sequences from HSC1 or MSCV. The retroviral regulatory elements may be from lentiviruses or they may be heterologous sequences identified from other genomic regions.

One skilled in the art would also appreciate that as other viral regulatory elements are identified, these may be used with the nucleic acid molecules of the invention.

### Polynucleotides of Interest/Therapeutic Nucleic Acid Molecules

Cells transfected or transduced in vitro can be used for ex vivo gene therapy or as a research tool or for protein production. The nucleic acid molecules are also useful for gene therapy by transfecting or transducing cells in vivo to express a therapeutic polynucleotide/protein in addition to modified tmpk. The therapeutic polynucleotide is alternatively referred to herein as the therapeutic cassette and/or therapeutic expression cassette. For example, if one were to upregulate the expression of a gene, one could insert the sense sequence into the nucleic acid molecule. If one were to downregulate the expression of the gene, one could insert the antisense sequence into the therapeutic expression cassette. Techniques for inserting sense and antisense sequences (or fragments of these sequences) would be apparent to those skilled in the art. The therapeutic nucleic acid molecule or nucleic acid molecule fragment may be either isolated from a native source (in sense or antisense orientations) or synthesized. It may also be a mutated native or synthetic sequence or a combination of these.

Examples of therapeutic coding nucleic acid molecules to be expressed include adenosine deaminase (ADA), γc interleukin receptor subunit, α-galactosidase A, acid ceramidase, galactocerebrosidase, and transmembrane conductance regulator (CFTR) molecules.

### Variations of Nucleic Acid Molecules

### Modifications

Many modifications may be made to the nucleic acid molecule DNA sequences including vector sequences disclosed in this application and these will be apparent to one skilled in the art. The invention includes nucleotide modifications of the sequences disclosed in this application (or fragments thereof) that are capable of directing expression or being expressed in mammalian cells. Modifications include substitution, insertion or deletion of nucleotides or altering the relative positions or order of nucleotides.

### Sequence Identity

The nucleic acid molecules of the invention also include nucleic acid molecules (or a fragment thereof having at least about: 70% identity, at least 80% identity, at least 90% identity, at least 95% identity, at least 96% identity, at least 97% identity, at least 98% identity or, most preferred, at least 99% or 99.5% identity to a nucleic acid molecule of the invention and which are capable of expression of nucleic acid molecules in mammalian cells. Identity refers to the similarity of two nucleotide sequences that are aligned so that the highest order match is obtained. Identity is calculated according to methods known in the art. For example, if a nucleotide sequence (called “Sequence A”) has 90% identity to a portion of [SEQ ID NO: 11], then Sequence A will be identical to the referenced portion of [SEQ ID NO: 11] except that Sequence A may include up to 10 point mutations (such as substitutions with other nucleotides) per each 100 nucleotides of the referenced portion of [SEQ ID NO: 11].

Sequence identity (each construct preferably without a coding nucleic acid molecule insert) is preferably set at least about: 70% identity, at least 80% identity, at least 90% identity, at least 95% identity, at least 96% identity, at least 97% identity, at least 98% identity or, most preferred, at least 99% or 99.5% identity to the sequences provided in SEQ ID NO:13 to SEQ ID NO:14 or its complementary sequence). Sequence identity will preferably be calculated with the GCG program from Bioinformatics (University of Wisconsin). Other programs are also available to calculate sequence identity, such as the Clustal W program (preferably using default parameters; Thompson, J D et al., Nucleic Acid Res. 22:4673-4680).

### Hybridization

The invention includes DNA that has a sequence with sufficient identity to a nucleic acid molecule described in this application to hybridize under stringent hybridization conditions (hybridization techniques are well known in the art). The present invention also includes nucleic acid molecules that hybridize to one or more of the sequences in [SEQ ID NO:11]-[SEQ ID NO:12] or its complementary sequence. Such nucleic acid molecules preferably hybridize under high stringency conditions (see Sambrook et al. Molecular Cloning: A Laboratory Manual, Most Recent Edition, Cold Spring Harbor Laboratory Press, Cold Spring Harbor, N.Y.). High stringency washes have preferably have low salt (preferably about 0.2% SSC) and a temperature of about 50-65° C. and are optionally conducted for about 15 minutes.

### Host Cells

The invention also relates to a host cell (isolated cell in vitro, a cell in vivo, or a cell treated ex vivo and returned to an in vivo site) containing a nucleic acid molecule of the invention. Cells transfected with a nucleic acid molecule such as a DNA molecule, or transduced with the nucleic acid molecule such as a DNA or RNA virus vector, are optionally used, for example, in bone marrow or cord blood cell transplants according to techniques known in the art. Examples of the use of transduced bone marrow or cord blood cells in transplants are for ex vivo gene therapy of Adenosine deaminase (ADA) deficiency. Other cells which are optionally transfected or transduced either ex vivo or in vivo include purified stem cells (of embryonic or later ontogeny), as described above.

### Pharmaceutical Compositions

The pharmaceutical compositions of this invention used to treat patients having diseases, disorders or abnormal physical states could include an acceptable carrier, auxiliary or excipient.

The pharmaceutical compositions are optionally administered by ex vivo and in vivo methods such as electroporation, DNA microinjection, liposome DNA delivery, and virus vectors that have RNA or DNA genomes including retrovirus vectors, lentivirus vectors, Adenovirus vectors and Adeno-associated virus (AAV) vectors, Semliki Forest Virus. Derivatives or hybrids of these vectors are also useful.

Dosages to be administered depend on patient needs, on the desired effect and on the chosen route of administration. The expression cassettes are optionally introduced into the cells or their precursors using ex vivo or in vivo delivery vehicles such as liposomes or DNA or RNA virus vectors. They are also optionally introduced into these cells using physical techniques such as microinjection or chemical methods such as coprecipitation.

The pharmaceutical compositions are typically prepared by known methods for the preparation of pharmaceutically acceptable compositions which are administered to patients, and such that an effective quantity of the nucleic acid molecule is combined in a mixture with a pharmaceutically acceptable vehicle. Suitable vehicles are described, for example in Remington's Pharmaceutical Sciences (Remington's Pharmaceutical Sciences, Mack Publishing Company, Easton, Pa., USA).

On this basis, the pharmaceutical compositions could include an active compound or substance, such as a nucleic acid molecule, in association with one or more pharmaceutically acceptable vehicles or diluents, and contained in buffered solutions with a suitable pH and isoosmotic with the physiological fluids. The methods of combining the expression cassettes with the vehicles or combining them with diluents is well known to those skilled in the art. The composition could include a targeting agent for the transport of the active compound to specified sites within cells.

### Method of Medical Treatment of Disease

Vectors containing the nucleic acid molecules of the invention are typically administered to mammals, preferably humans, in gene therapy using techniques described below. The polypeptides produced from the nucleic acid molecules are also optionally administered to mammals, preferably humans. The invention relates to a method of medical treatment of a mammal in need thereof, preferably a human, by administering to the mammal a vector of the invention or a cell containing a vector of the invention. A recipient, preferably human, who develops an adverse event, such as graft versus host disease, is typically administered a drug, such as AZT, that is a substrate for the modified tmpk molecules of the invention. Diseases, such as blood diseases or neural diseases (neurodegenerative), that are readily treated are described in this application and known in the art (eg. diseases, such as thalassemia or sickle cell anemia that are treated by administering a globin gene as described in Canadian patent application no. 2,246,005). Blood diseases treatable by stem cell transplant include leukemias, myelodysplastic syndromes, stem cell disorders, myeloproliferative disorders, lymphoproliferative disorders phagocyte disorders, inherited metabolic disorders, histiocytic disorders, inherited erythrocyte abnormalities, inherited immune system disorders, inherited platelet abnormalities, plasma cell disorders, malignancies (See also, Medical Professional's Guide to Unrelated Donor Stem Cell Transplants, 4th Edition). Stem cell nerve diseases to be treated by neural stem cell transplantation include diseases resulting in neural cell damage or loss, eg. paralysis, Parkinson's disease, Alzheimer's disease, ALS, multiple sclerosis). The vector of the invention is useful as a stem cell marker and to express genes that cause stem cells to differentiate (e.g. growth factor).

### Gene Therapy

The invention includes compositions and methods for providing a coding nucleic acid molecule to a subject such that expression of the molecule in the cells provides the biological activity of the polypeptide encoded by the coding nucleic acid molecule to those cells. A coding nucleic acid as used herein means a nucleic acid that comprises nucleotides which specify the amino acid sequence, or a portion thereof, of the corresponding protein. A coding sequence may comprise a start codon and/or a termination sequence.

The invention includes methods and compositions for providing a coding nucleic acid molecule to the cells of an individual such that expression of the coding nucleic acid molecule in the cells provides the biological activity or phenotype of the polypeptide encoded by the coding nucleic acid molecule. The method also relates to a method for providing an individual having a disease, disorder or abnormal physical state with a biologically active polypeptide by administering a nucleic acid molecule of the present invention. The method may be performed ex vivo or in vivo. Gene therapy methods and compositions are demonstrated, for example, in U.S. Pat. Nos. 5,869,040, 5,639,642, 5,928,214, 5,911,983, 5,830,880, 5,910,488, 5,854,019, 5,672,344, 5,645,829, 5,741,486, 5,656,465, 5,547,932, 5,529,774, 5,436,146, 5,399,346 and 5,670,488, 5,240,846. The amount of polypeptide will vary with the subject's needs. The optimal dosage of vector may be readily determined using empirical techniques, for example by escalating doses (see U.S. Pat. No. 5,910,488 for an example of escalating doses).

Various approaches to gene therapy may be used. The invention includes a process for providing a human with a therapeutic polypeptide including: introducing human cells into a human, said human cells having been treated in vitro or ex vivo to insert therein a vector of the invention, the human cells expressing in vivo in said human a therapeutically effective amount of said therapeutic polypeptide.

The method also relates to a method for producing a stock of recombinant virus by producing virus suitable for gene therapy comprising modified DNA encoding globin. This method preferably involves transfecting cells permissive for virus replication (the virus containing modified globin) and collecting the virus produced.

Cotransfection (DNA and marker on separate molecules) may be employed (see eg U.S. Pat. No. 5,928,914 and U.S. Pat. No. 5,817,492). As well, a detection cassette or marker (such as Green Fluorescent Protein marker or a derivative, CD19 or CD25) may be used within the vector itself (preferably a viral vector).

### Polypeptide Production and Research Tools

A cell line (either an immortalized cell culture or a stem cell culture) transfected or transduced with a nucleic acid molecule of the invention (or variants) is useful as a research tool to measure levels of expression of the coding nucleic acid molecule and the activity of the polypeptide encoded by the coding nucleic acid molecule.

The nucleic acid molecules are useful in research to deliver marker genes or antisense RNA to cells.

The invention includes a method for producing a recombinant host cell capable of expressing a nucleic acid molecule of the invention comprising introducing into the host cell a vector of the invention.

The invention also includes a method for expressing a polypeptide in a host cell of the invention including culturing the host cell under conditions suitable for coding nucleic acid molecule expression. The method typically provides the phenotype of the polypeptide to the cell.

In these methods, the host cell is optionally a stem cell or a T cell.

Another aspect of the invention is an isolated polypeptide produced from a nucleic acid molecule or vector of the invention according to a method of the invention.

### Uses of Tmpk

Also provided are a number of uses of tmpk. All the aforementioned tmpk variants, delivery vectors, detection cassettes, therapeutic genes, methods and composition embodiments are contemplated for the various uses herein described.

One embodiment provides use of a vector construct comprising a stably integrating delivery vector; and a modified mammalian thymidylate kinase (tmpk) polynucleotide for expressing a modified mammalian tmpk polynucleotide in a mammalian cell or subject.

Another embodiment provides a vector construct comprising a stably integrating delivery vector; and a modified mammalian thymidylate kinase (tmpk) polynucleotide for expressing a modified mammalian tmpk polynucleotide in a mammalian cell or subject.

A further embodiment provides use of a vector construct comprising a stably integrating delivery vector; and a modified mammalian thymidylate kinase (tmpk) polynucleotide in the manufacture of a medicament for expressing a modified mammalian tmpk polynucleotide in a mammalian cell or subject.

Another embodiment provides use of a composition comprising a stably integrating delivery vector and a modified mammalian thymidylate kinase (tmpk) polynucleotide for expressing a modified mammalian tmpk polynucleotide in a mammalian cell or subject.

A further embodiment provides a composition comprising a stably integrating delivery vector; and a modified mammalian thymidylate kinase (tmpk) polynucleotide for expressing a modified mammalian tmpk polynucleotide in a mammalian cell or subject.

Yet a further embodiment provides use of a composition comprising a stably integrating delivery vector; and a modified mammalian thymidylate kinase (tmpk) polynucleotide in the manufacture of a medicament for expressing a modified mammalian tmpk polynucleotide in a mammalian cell or subject.

Another embodiment, provides use of a vector construct or composition comprising a stably integrating delivery vector; and a modified mammalian thymidylate kinase (tmpk) polynucleotide for gene therapy.

A further embodiment provides a vector construct or composition comprising a stably integrating delivery vector; and a modified mammalian thymidylate kinase (tmpk) polynucleotide for gene therapy.

Yet a further embodiment provides a vector construct or composition comprising a stably integrating delivery vector; and a modified mammalian thymidylate kinase (tmpk) polynucleotide for the manufacture of a medicament for gene therapy.

Also provided is use of a vector construct or composition disclosed herein for treating a disease selected from the group consisting of cancer, GVHD or diseases resulting from a deficiency of a gene product.

Another embodiment provides a vector construct or composition disclosed herein for treating a disease selected from the group consisting of cancer, GVHD or diseases resulting from a deficiency of a gene product.

A further embodiment provides a vector construct or composition disclosed herein for the manufacture of a medicament for treating a disease selected from the group consisting of cancer, GVHD or diseases resulting from a deficiency of a gene product.

Another aspect provides use of an effective amount of a prodrug for killing a cell expressing a modified mammalian tmpk polynucleotide wherein the expression of the modified mammalian tmpk results from contact with a vector construct or composition comprising a stably integrating delivery vector; and a modified mammalian thymidylate kinase (tmpk) polynucleotide.

One embodiment provides an effective amount of a prodrug for killing a cell expressing a modified mammalian tmpk polynucleotide wherein the expression of the modified mammalian tmpk results from contact with a vector construct or composition comprising a stably integrating delivery vector; and a modified mammalian thymidylate kinase (tmpk) polynucleotide.

Yet another embodiment provides use of an effective amount of a prodrug for the manufacture of a medicament for killing a cell expressing a modified mammalian tmpk polynucleotide wherein the expression of the modified mammalian tmpk results from contact with a vector construct or composition comprising a stably integrating delivery vector; and a modified mammalian thymidylate kinase (tmpk) polynucleotide.

The following non-limiting examples are illustrative of the present invention:

### EXAMPLES

The following non-limiting examples are illustrative of the present invention:

### Example 1

### Materials and methods

cDNA Cloning of Human CD19 and Construction of Shuttle Vector

Full-length human CD19 (hCD19) cDNA was obtained by reverse transcriptase-polymerase chain reaction (RT-PCR) from the human Burkitt's lymphoma cell line (Raji) using primers CD19 F1 and CD19 R1 described below. The cloned PCR product was directly ligated into the TA-vector, pPCR-script SK(+)(Stratagene) to give pPCR-CD19full. A truncated form of hCD19 (CD19D), which has only the extracellular and transmembrane domains, but lacks the cytoplasmic domain, was generated by inverse-PCR from pPCR-script SK(+)-CD19 using primers CD19 F2 and CD19 R2 described below, to give pPCR-CD19D. Following the sequence confirmation of cDNA inserts in pPCR-script SK(+)-CD19D, the cloned cDNA fragments were then subcloned into the EcoRI site of the shuttle vector pSV-IRES to give pSV-IRES-CD19D. The primer sequences used for cloning of human CD19 cDNA as follows: CD19 F1: 5′-atgccacctcctcgcctcctcttcttcc-3′ (SEQ ID NO: 23) and CD19 R1: 5′-tcacctggtgctccaggtgccc-3′ (SEQ ID NO: 24). The truncated construct was made by inverse-PCR using primers CD19 F2: 5′-ccgccaccgcggtggagctccag-3′ (SEQ ID NO: 25) and CD19 R2: 5′-ttaaagatgaagaatgcccacaaggg-3′ (SEQ ID NO: 26).

cDNA Cloning of Human Thymidylate Kinase, Construction of Bicistoronic Lentiviral Expression Vectors and Preparation of High-Titer Virus

To clone wild-type human thymidylate monophosphate kinase (tmpk) cDNA, peripheral blood mononuclear cells (PBMNCs) were isolated from heparinized blood obtained from healthy human donors by Ficoll-Hypaque (Amersham-Pharmacia) separations. Wild-type human tmpk cDNA was amplified by PCR using first strand complementary DNA (cDNA) generated by reverse-transcription from total RNA extracted from the PBMNCs using TRIZOL reagent (Invitrogen). PCR products for wild-type tmpk and each modified version of human tmpk cDNA, such as R200A, F105Y, and R16GLL, which was constructed by Dr. A. Lavie at the University of Illinois at Chicago, were subcloned into pPCR-scriptSK(+) and sequenced. Following the sequence confirmation, each cDNA was first subcloned into shuttle vector pSV-IRES-CD19D to construct a bicistronic cassette consisting the suicide gene, internal ribosomal entry site (IRES) derived from encephalomyocardiTUS virus (EMCV), and the truncated form of human CD19. This bicistronic expression cassette with tmpk and hCD19, flanked by an EMCV IRES. Then subcloned into HIV-1 based recombinant lentiviral plasmid vector used in the production of lentivirus, pHR′-cPPT-EF-W-SIN (pHR′). The expression of gene of interests was controlled by the internal EF1a promoter. As a control for the transduction experiments, the inventors used both pHR′-cPPT-EF-IRES-hCD19-W-SIN and pHR′-cPPT-EF-EGFP-W-SIN vectors carrying either IRES-hCD19 or the enhanced GFP (EGFP) cDNAs, respectively.

VSVG-pseudotyped LVs, including an EGFP marking vector (LV-EGFP), were generated by transient transfection of 293T cells (kindly provided by Dr. Robert Pawliuk, Division of Health Sciences and Technology, MIT, Cambridge, Mass.) using the three-plasmid system (the aforementioned LV plasmid constructs, the packaging plasmid pCMVΔR8.91, and the VSVG envelope encoding plasmid pMD.G). The transfections were performed with either FuGENE6 (Roche Applied Science, Indianapolis, Ind.) or CaPO4-precipitation methods. Viral supernatants were harvested 48 h later and concentrated by ultracentrifugation at 50,000×g for 2 h at 4° C. The concentrated viral supernatants were serially diluted and titered on 293T cells (ATCC, Manassas, Va.). Table 1 lists the titers of virus used in these experiments. Flow cytometric analyses were performed 72 h later using a FACSCalibur (BD Biosciences, San Jose, Calif.) for evaluating EGFP or hCD19 expression after staining with monoclonal PE-conjugated mouse anti-human CD19. Titers are expressed as infectious particles (IP)/mL.

TABLE 1
Titer of LVs on 293T cells used in this study

| Transgene | Detection | Titer (IP/mL) |
|---|---|---|
| EGFP | EGFP | 1.4 × 10⁸ |
| Tmpk (wild-type)-IRES-hCD19 | CD19 | 2.3 × 10⁸ |
| Tmpk (R200A)-IRES- hCD19 | CD19 | 3.5 × 10⁸ |
| Tmpk (F105Y + R200A)-IRES- hCD19 | CD19 | 5.9 × 10⁸ |
| Tmpk (R16G Large Lid)-IRES- hCD19 | CD19 | 1.5 × 10⁹ |
| IRES- hCD19 | CD19 | 1.4 × 10⁹ |

### Transduction and Analysis of Transgene Expression by Flow Cytometric Analysis.

Human T lymphoma cell line, Jurkat, and human erythro-leukemic cell line, K562, were maintained in RPMI 1640 supplemented with 10% FBS, 100 U/ml of penicillin, and streptomycin to 100 μg/ml. Cells were infected with concentrated virus stocks using an MOI of 10 in the presence of 8 μg/ml protamine sulfate. Infected cells were then kept in culture for 5 days prior to evaluating gene transfer efficiency. Gene transfer efficiencies were measured by flow cytometry using a monoclonal anti-human CD19-antibody conjugated with phycoerythrin (PE). About 10⁶ non-transduced and virally transduced cells were incubated for 15 min with the antibody or the corresponding IgG₁ isotype control antibody at 4° C. Cells were washed with phospahate-buffered saline (PBS). Cell analysis was performed on a FACS Calibur and data were analyzed using Cell Quest software. Single-cell clones were obtained by limiting dilution and clones with the highest expression of CD19 were selected.

### Western Blot Analysis of Tmpk-Overexpression by LV-Transduction in Jurkat Cells.

Tmpk overexpression in the infected cells were examined by Western blot analysis using rabbit anti-human tmpk antibody (gift from Dr. Manfred Konrad, Maxplank Institute) as well as mouse anti-human beta-actin as an internal control for the blot. Total cell lysates were resolved by 12% SDS-polyacrylamide gels (SDS-PAGE) and transferred onto polyvinylidene difluoride filters (Millipore, Billerica, Mass.). Filters were blocked with 5% fat free skim milk in Tris-buffered saline (TBS) with 0.05% Tween 20 (TBST) for 1 hr at room temperature. Human tmpk overexpression was elucidated using rabbit anti-human tmpk antiserum, diluted to 1 in 5000. Protein loading amounts in each well was confirmed with an anti-beta actin antibody diluted 1:5000. Blots were probed with a secondary anti-rabbit IgG (diluted 1:5000) or anti-mouse IgG (diluted 1:5000) horseradish peroxidase-conjugated antibodies, and protein bands were detected using an enhanced chemiluminesence kit (Perkin Elmer, Norwalk, Conn.) and Kodak BioMAX XAR film.

Comparison of Transduction Efficiencies and hCD19 Expression Levels in LV-Transduced Jurkat Cells.

Cells were infected with concentrated virus stocks using an MOI of 10 in the presence of 8 μg/ml protamine sulfate. Infected cells were then kept in culture for 5 days prior to evaluating gene transfer efficiency. Gene transfer efficiencies were measured by flow cytometry using a monoclonal anti-human CD19-antibody conjugated to phycoerythrin (PE). About 10⁶ non-transduced and virally transduced cells were incubated for 15 min with the antibody or the corresponding IgG₁ isotype control antibody at 4° C. Cells were washed with phospahate-buffered saline (PBS). Cell analysis was performed on a FACS Calibur and data were analyzed using Cell Quest software. Single-cell clones were obtained by limiting dilution, and clones with the highest expression of CD19 were selected. Percentages indicate EGFP or CD19 expression and mean fluorescence intensity (MFI) values indicate the levels of expression levels in the cells.

Determination of AZT-Sensitivity of Jurkat (Human T Cell Line) Transduced with LV-Tmpk-IRES-hCD19 and Mutant Forms.

Transduced Jurkat cells and the single-cell clones were seeded in 96 well plates (2×10⁵/well) in 200 μl of medium containing increasing concentrations of AZT (0, 1, 10 and 100 μM). The medium was changed daily. After 4 days of culture, cell viability was determined by MTT assay (Promega). **, P<0.01, n=3. Data are expressed as mean±standard error of mean (SEM).

### Induction of Apoptosis by Addition of 100 μM AZT in LV-Tmpk-Transduced Jurkat Cells.

Cells were seeded in 24 well plates (10⁶/well) in 1 ml of medium with or without 100 μM of AZT. The medium was changed daily. After 4 days of culture, induction of apoptosis in the cells were analyzed by annexin-V staining according to the manufacturer's protocol (Annexin V-APC: BD Pharmingen). **, P<0.01, n=3. Data are expressed as mean±SEM.

Determination of AZT-Metabolites in the Cells Treated with 100 μM AZT.

The cells were cultured in the presence of 100 μM AZT for 36 hrs. 10⁷ cells were homogenized by sonication in 100 μl of 5% (w/v) trichloroacetic acid. The supernatant is collected after homogenate had been centrifuged at 10,000×g for 15 min at 4° C. The trichloroacetic acid was removed by extraction with an equal volume of 20% tri-n-octylamine in pentane. The neutralized aqueous fraction is directly injected into HPLC. Separation of AZT and its metabolites was performed on a C18 column (Waters) with a mobile phase composed of 0.2 M phosphate buffer containing 4 mM tetrabutylammonium hydrogen sulfate (pH 7.5) and acetonitrile in the ratio of 97:3 (v/v). The mobile phase was pumped at a flow rate of 1.5 ml/min. The UV absorbance was monitored at 270 nm. Five million cell equivalents were injected and analyzed in triplicate.

### AZT-Mediated Loss of Mitochondrial Function is Induced by Expression of TMPK-LargeLid.

Cells (10⁶ cells) treated with (shown (+) in figure) or without (−) 100 μM AZT were stained with JC-1 for 15 min at 37° C., and then were analyzed by flow cytometry. ***, P<0.001, n=3.

### Cellular Proliferation is not Always a Prerequisite for AZT-Induced Apoptosis.

Cells were seeded in 24-well plates (10⁶/well) in 1 ml of medium containing 0 (shown in AZT (−) in figure) or 100 μM of AZT (shown in AZT (+)) with or without 5 μM indirubin-3′-oxime (Figure (B) and (A), respectively). The medium was refreshed daily. After 2 days of culture, induction of apoptosis by AZT was analyzed by annexin V staining according to the manufacturer's protocol described. **, P<0.01, n=3. Data are expressed as mean±SEM.

Mutant Forms of Tmpk Prevent Growth of Transduced K562 Cells Xenografted into AZT-Treated NOD/SCID Mice.

Female or male 5 to 8-week-old non-obese diabetic/severe combined immunodeficient (NOD/SCID) mice were purchased from Jackson Laboratory. Lentivirally-transduced or non-transduced K562 cells (20×10⁶ cells) were resuspended in 0.5 mL Dulbecco's phosphate-buffered saline (D-PBS) per inoculum and injected subcutaneously (SC) into the right flanks of recipient mice. AZT treatment, which was administered intraperitoneally (IP) at the dose of 2.5 mg/kg/day, was started one day after injection and conducted for 14 days. In vivo tumor cell growth was monitored by measuring tumor size for up to 32 days post-inoculations. All experimental data were reproduced at least twice.

### Transduction of Primary Cultured Human or Mouse T Cells and Analysis of Transgene Expression

Human T lymphocytes are obtained from peripheral blood mononuclear cells (PBMNCs) isolated from heparinized blood obtained from healthy human donors by Ficoll-Hypaque (Amersham-Pharmacia) separations. Mouse T are prepared from the spleen following B cell depletion using goat anti-mouse IgG beads. T cells are activated by using anti-CD3 and anti-CD28 coated beads in a ratio of 1:3 (cell:beads) with 20 IU/mL of recombinant human interleukin 2 for 3 days. Cells were infected with concentrated virus stocks using an MOI of indicated in the presence of 8 μg/ml protamine sulfate. Infected cells were then kept in culture for 5 days prior to evaluating gene transfer efficiency. Gene transfer efficiencies were measured by flow cytometry using a monoclonal anti-human CD19-antibody conjugated with phycoerythrin (PE). About 10⁶ non-transduced and virally transduced cells were incubated for 15 min with the antibody or the corresponding IgG₁ isotype control antibody at 4° C. Cells were washed with phospahate-buffered saline (PBS). Cell analysis was performed on a FACS Calibur and data were analyzed using Cell Quest software.

### Statistical Analysis

Statistical analyses was performed using Instat 2.00 (GraphPad). The unpaired Student's t test was used to determine statistical significance. In some experiments, a one-way analysis of variance (ANOVA) with a Bonferroni post-test was used to determine statistically significant results.

### Example 2

### Generation and Titration of Tmpk cDNA Carrying Lentiviral Vectors

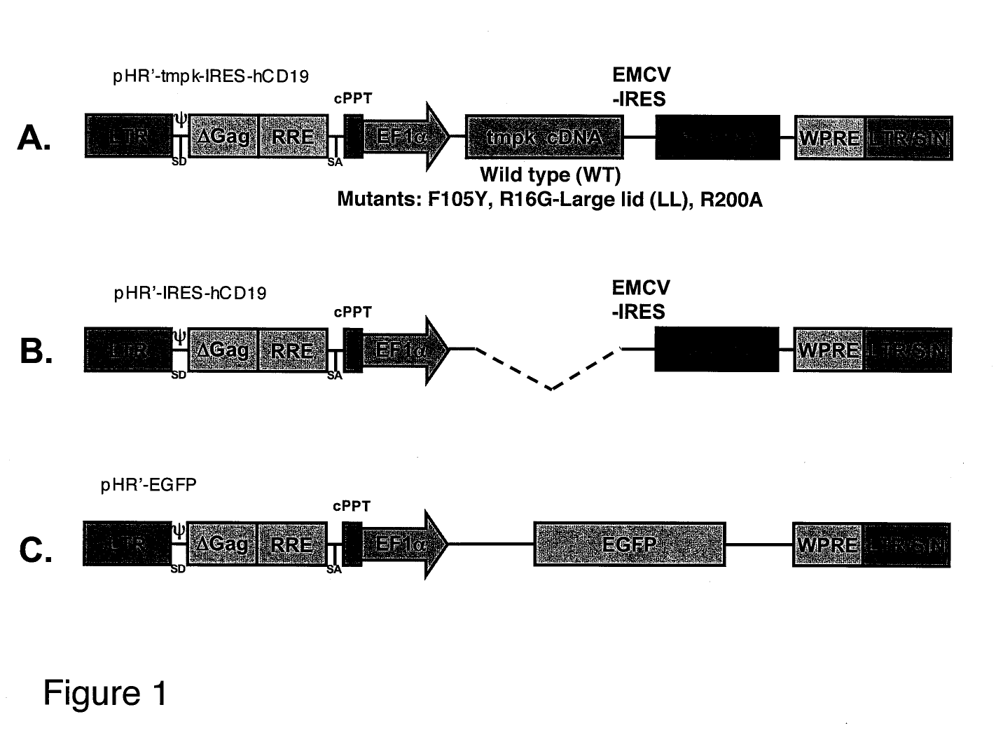

Two bi-cistronic lentiviral vectors with either wild-type or mutant human tmpk cDNA located at the upstream of EMCV-IRES sequence and mutant form of human CD19 which was deleted intracellular domain were constructed (FIG. 1). These vectors were derived from LV-EGFP which is a lentiviral vector expressing enhanced GFP under the control of the internal elongation factor-alpha (EF1-a) promoter. The virus titers obtained for each transfer vector were shown in Table 1.

It is known that the expression level of downstream gene by IRES-dependent manner in the bicistronic vector is in between 20 to 50% of that of upstream gene. However, the IRES-dependent expression of downstream gene also depends on the cell-type. While no CD19-expression was seen in transduced-HeLa cells, the expression was detected in the transduced 293T cells. We, however, could detect EGFP expression in the transduced HeLa cells as well as that in 293T cells. These data indicate that when the inventors used the IRES-element for expressing the gene of interests on both upstream and down stream of IRES-sequence in lentiviral system, the inventors need to use 293T cells to measure the functional titer of the virus.

### Example 3

### Transduction of Jurkat Cells with Recombinant Lentiviruses

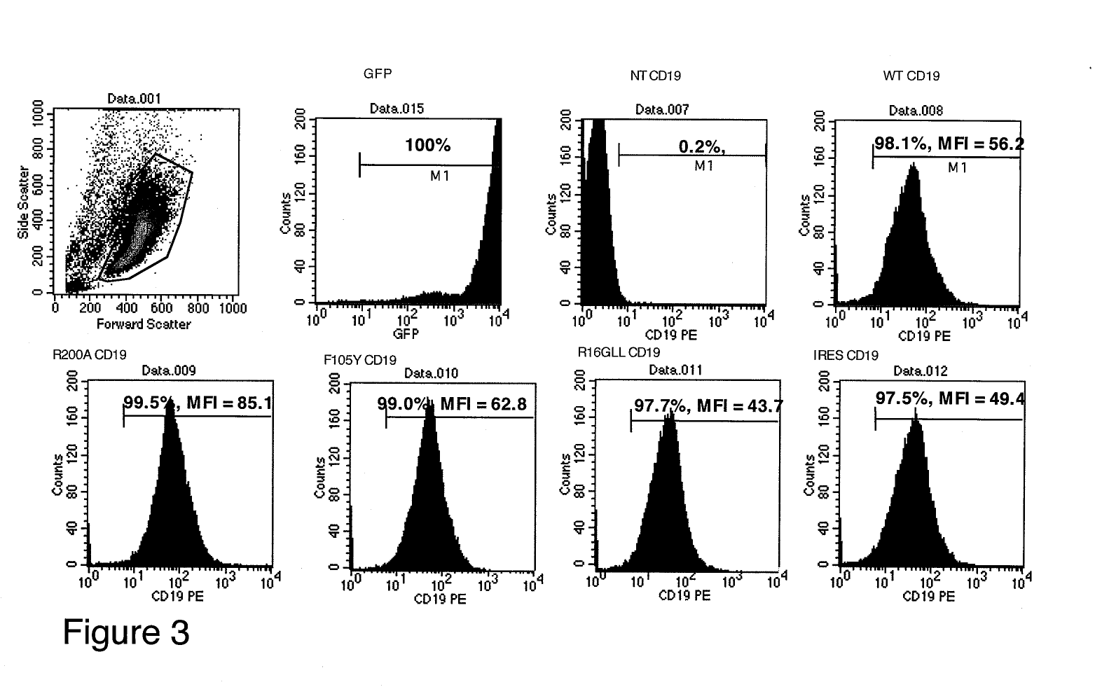

To compare the cell killing activity of each LV-constructs expressing tmpk cDNA, the inventors transduced human leukemia cell line Jurkat cells with using an MOI of 10 for 24 hrs. After 5 days of transduction, the inventors tested the CD19 expression in the transduced cells. While no CD19 expression was observed in non-transduced cells, strong CD19-expression was detected on each LV-transduced cells (FIG. 3). The mean fluorescent intensity of CD19 in each LV-transduced cells showed almost same levels indicates that that each LV-transduced cell expressed CD19 in a similar level.

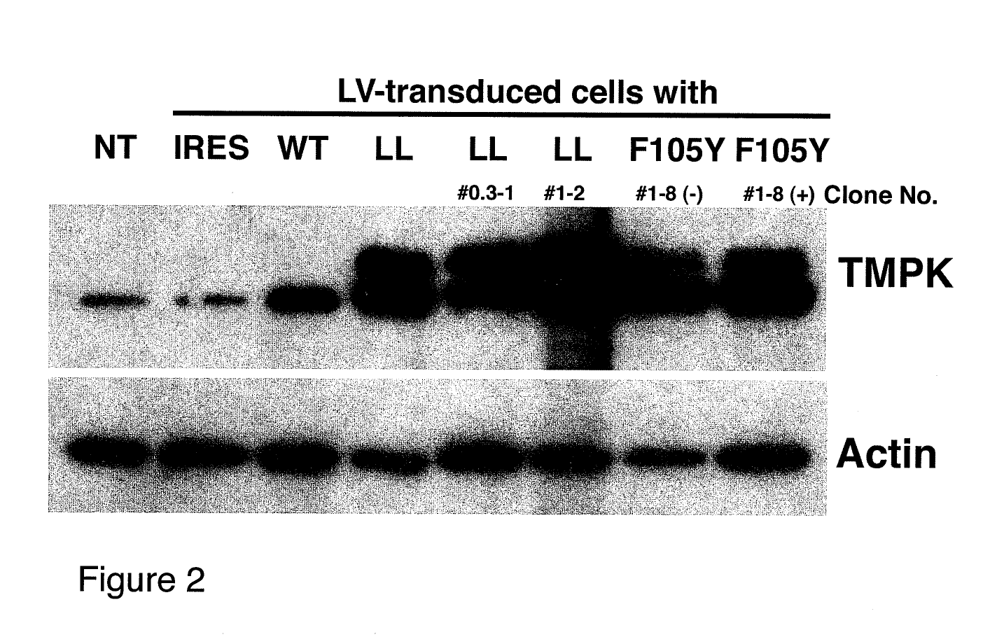

To test the expression levels of the upstream gene in each LV-construct, the inventors examined Western blot analysis using both rabbit anti-human tmpk as well as rabbit anti-human beta actin as an internal control. Since tmpk is expressed endogenously in the non-transduced Jurkat cells, the inventors could see the tmpk-gene expression in the cells. Comparing the LV-IRES-hCD19-transduced cells and non-transduced cells, LV-tmpk (wild-type; WT)-IRES-hCD19 or LV-tmpk-mutant cDNA-IRES-hCD19-transduced cells showed an increase of tmpk expression in the cells up to 10 times (FIG. 2).

### Example 4

### Measure AZT-Sensitivity of the Transduced Cells

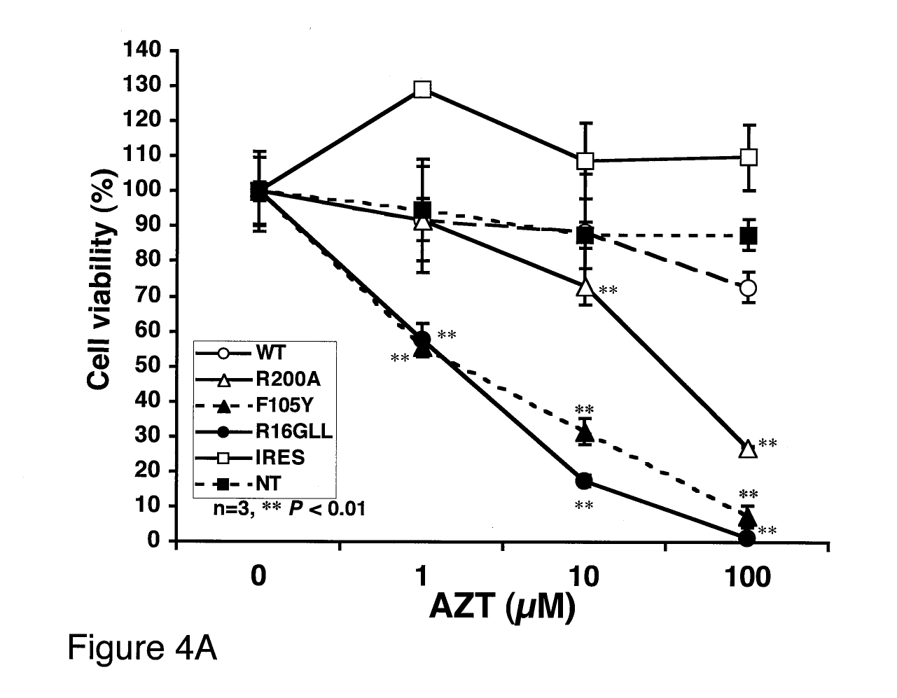

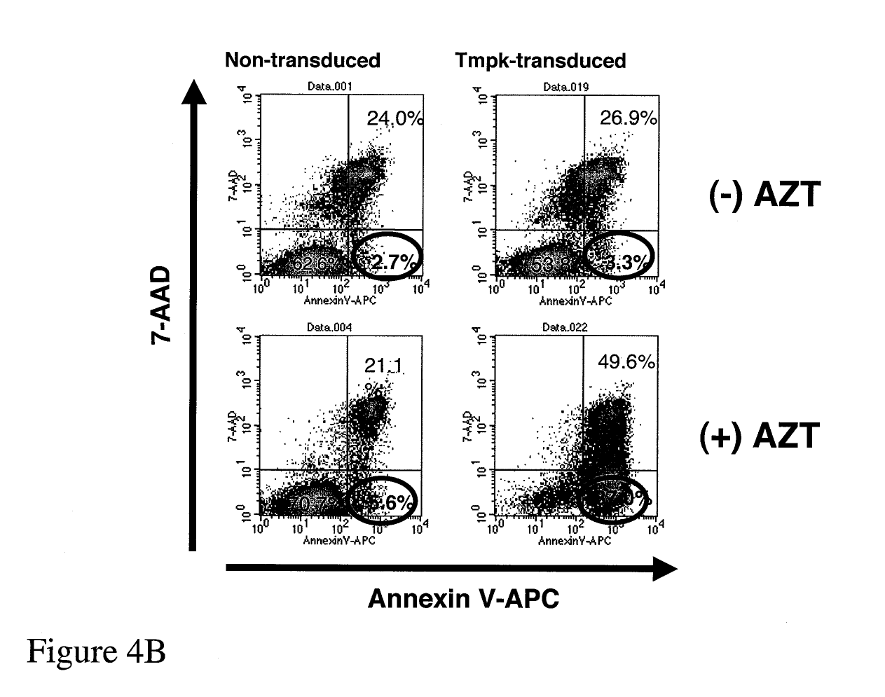

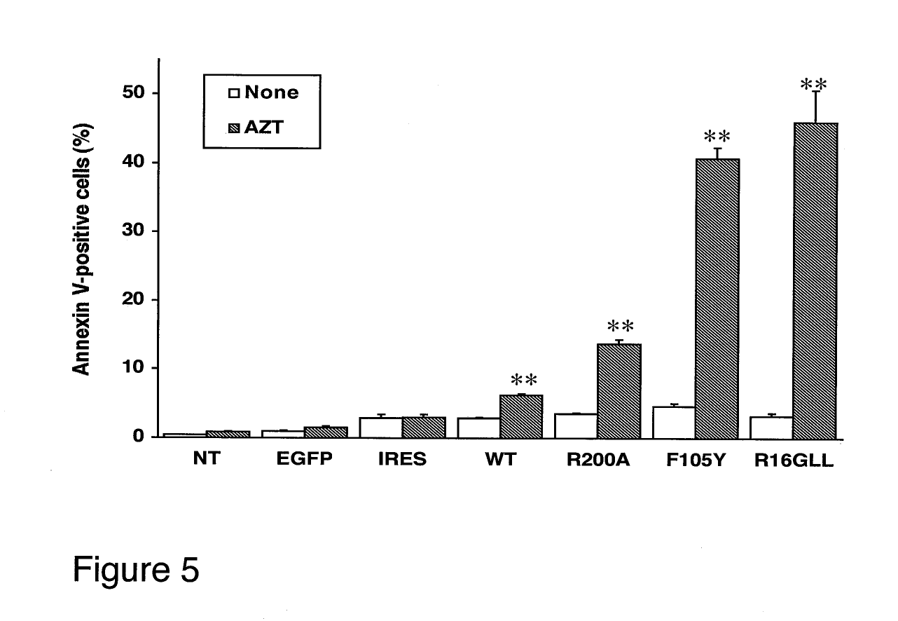

To examine the AZT-mediated celikilling activity of tmpk cDNA, each of transduced cells were incubated with the increasing concentration of AZT. After incubating both non-transduced and LV-transduced cells with AZT for 5 days, cell viability was determined using MTT assay (FIG. 4A). These transduced cells were efficiently and selectively killed in a dose-dependent manner by AZT (IC₅₀ of 2 μM), while wild-type tmpk transduced cells were non-sensitive to AZT up to 100 μM. Among of them, both LV-tmpk F105Y and LV-tmpk R16GLL transduced cells showed the dose-dependent cell killing activity. Since MTT assay reflects mitochondrial enzymatic activities in living cell to metabolize the MTT-assay substrate, AZT-metabolites supposed to inhibit mitochondrial function and induced cellular death. To confirm the induction of cellular death such as an apoptosis, the inventors next examined the induction of apoptosis following AZT-treatment in the tmpk-expressing cells by flow cytometric analysis following the annexin V-staining of the cells. In response to AZT treatment, the early apoptotic cell indices of cells transduced with wild-type tmpk, F105Y or LL were 6.2±0.3%, 40.7±1.7%, and 46.1±4.6%, respectively (n=3). No induction of apoptosis by AZT was observed in the group of negative control group including non-transduced cells and IRES (FIG. 5). In contrast, significant increases in the apoptosis-induced cells were observed in the LV-tmpk transduced cells following AZT-treatment.

### Example 5

### Intracellular AZT Metabolite Concentration

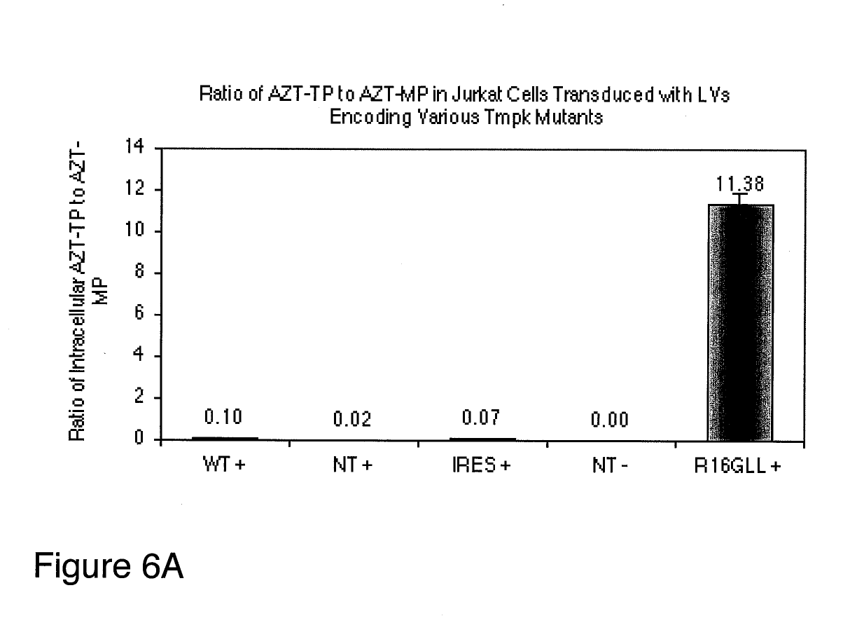

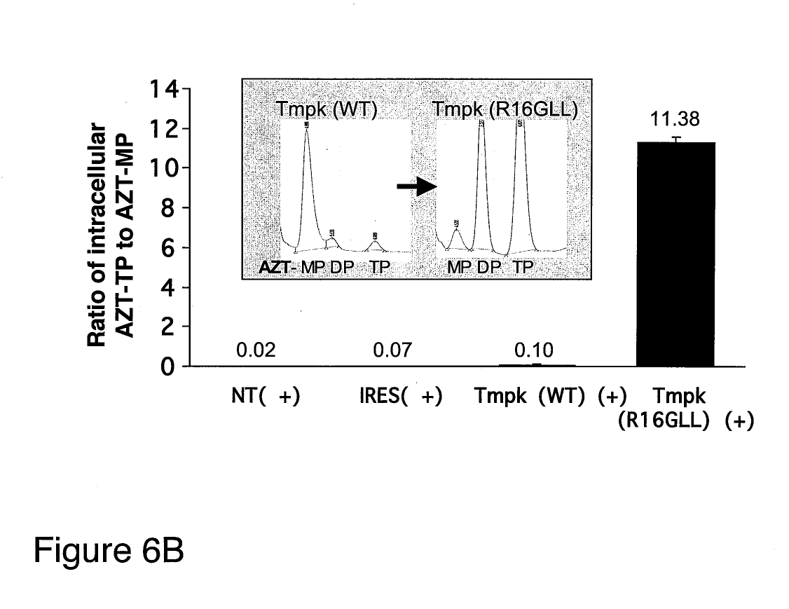

To evaluate the intracellular concentration of AZT-antimetabolites in the cells, the inventors have established by HPLC. After treatment of the cells transduced with the tmpk LL with AZT, they efficiently convert AZT into the active antimetabolite form, AZT-triphosphate (AZT-TP) (conversion ratio of AZT-TP to AZT MP 11.3 compared to 0.02 in non-transduced cells) (FIG. 6). Conversion of AZT-TP by cells transduced with wild-type tmpk (conversion ratio of 0.10) is only marginally better than the conversion in non-transduced cells (FIG. 6).

### Example 6

### AZT-Mediated Loss of Mitochondrial Function is Induced by Expression of TMPK-LargeLid

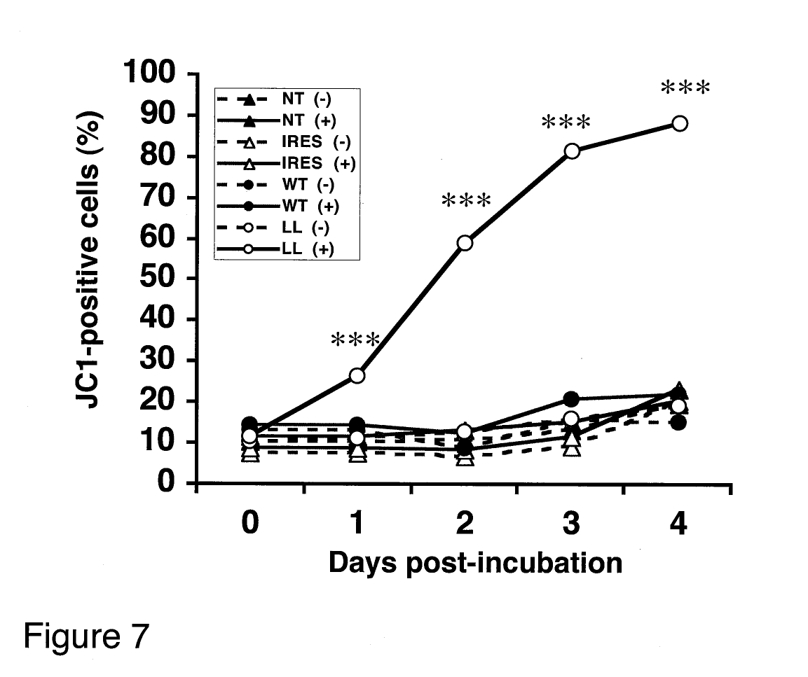

AZT is a potent inhibitor of HIV replication. However, many patients treated with AZT develop toxic mitochondrial myopathy. Long-term AZT treatment has been shown to induce mitochondrial biochemical dysfunction in AIDS patients. In order to prove the mechanism of the induction of cellular apoptosis after AZT-treatment in the tmpk-transduced cells, the inventors measured the membrane potential of mitochondria by analyzing the decrease of the percentage of red-fluorescence in the flow diagram followed by staining the cells with JC-1 reagent. A significant increase in the loss of mitochondrial membrane potential (Δψ) was found to occur in the LV-tmpk R16GLL transduced cells after AZT-treatment in a time dependent manner, however, negative control cell group cells did not increase the percentage of the mitochondrial membrane potential lose cells (FIG. 7).

### Example 7

### AZT/Tmpk Mediated Cell Killing Does Not Need Cellular Proliferation

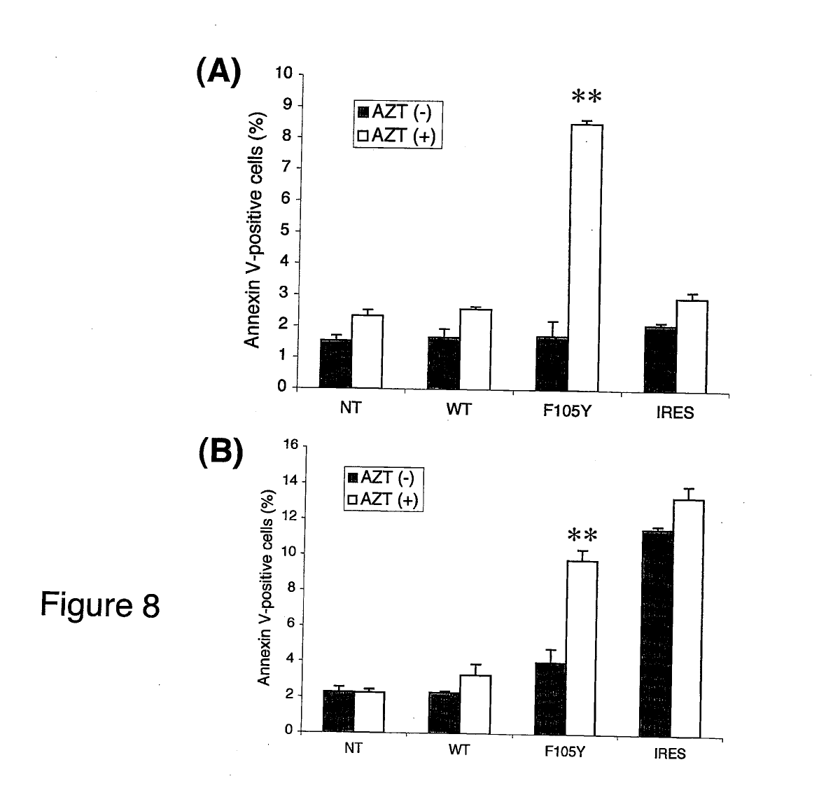

HSV1-tk mediated cell killing requires cellular proliferation for the cytotoxic effect. Here, the inventors have shown proliferation-independent cell killing using mutant tmpk and AZT. Indirubin-metabolites work as cyclin-dependent kinase inhibitors, which function by competing with ATP for binding to the catalytic subunit. They lead to G2/M arrest in many cell lines and G1/S arrest in Jurkat cells. Indirubin-3′-oxime was used to arrest cell cycling, and then transduced cells were treated with AZT. Only 2% of cells transduced with wild-type tmpk were killed, whereas the inventors attained 20% killing of cells transduced with LV-tmpkF105Y-IRES-hCD19 (FIG. 8).

### Example 8

### In Vivo Tumor Killing Effect Using the Tmpk-Transduced K562-Xenografted NOD/SCID Mouse Model

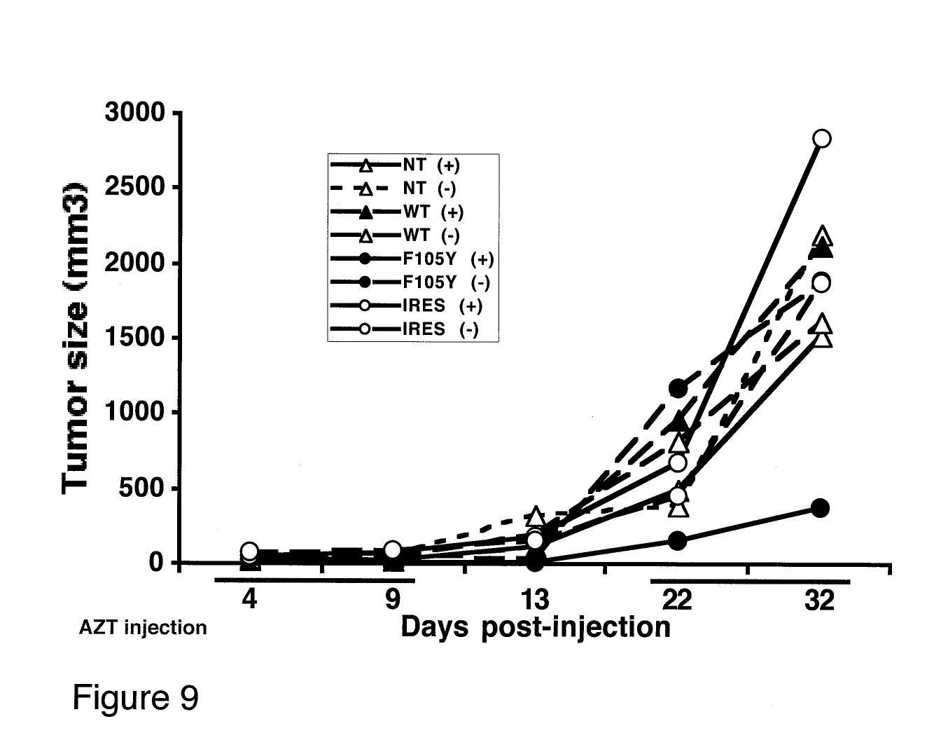

The inventors next addressed the cell killing ability of the various tmpk mutants in an in vivo tumor model. K562 erythro-leukemia cells were transduced with either wild-type tmpk or the F105Y mutant and injected subcutaneously into NOD/SCID mice. Mice were then treated with 2.5 mg/kg of AZT for the following two-weeks. Non-transduced K562 cells gave rise to tumors of an average 2000 mm² in size at four and a half weeks past injection. Strikingly, while no significant reduction in tumor volume was apparent in AZT-treated mice injected with K562 cells transduced with wild-type tmpk (2000 mm² on average), the inventors have observed a 6 to 20 fold reduction in tumor volume in mice that were injected with K562 cells transduced with the F105Y tmpk mutant following AZT treatment (100-300 mm² final tumor volume consisting primarily of non-transduced K562 cells) (FIG. 9).

### Example 9

### Transduction of Primary Cultured Human or Mouse T Cells

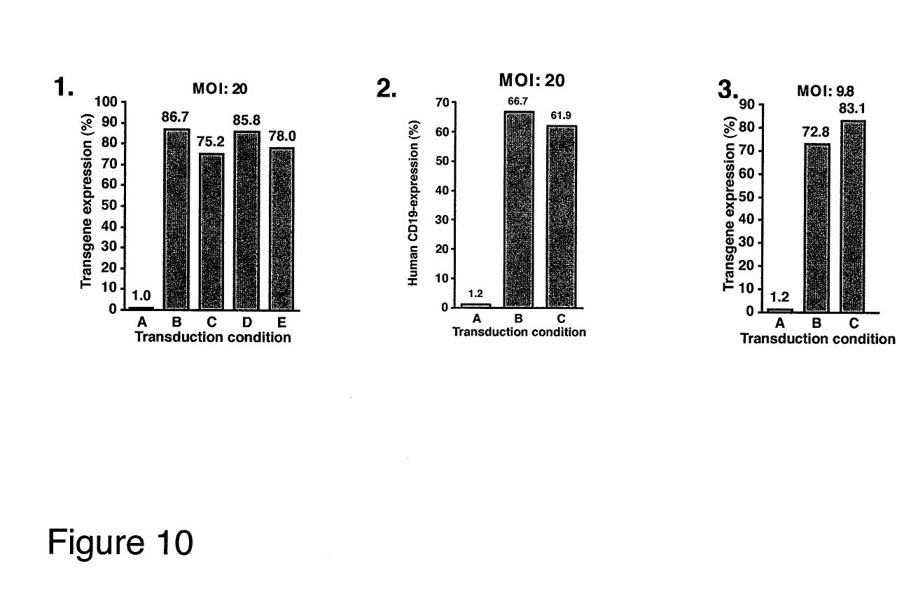

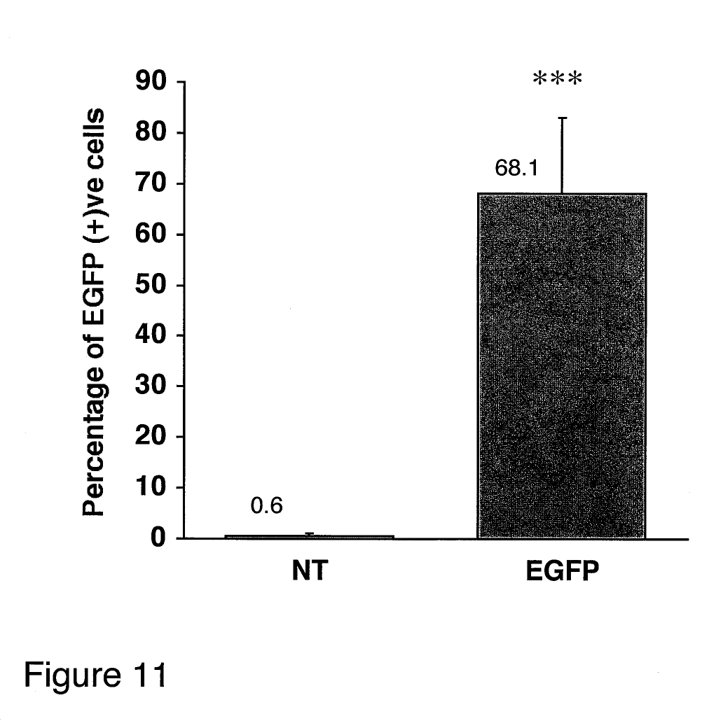

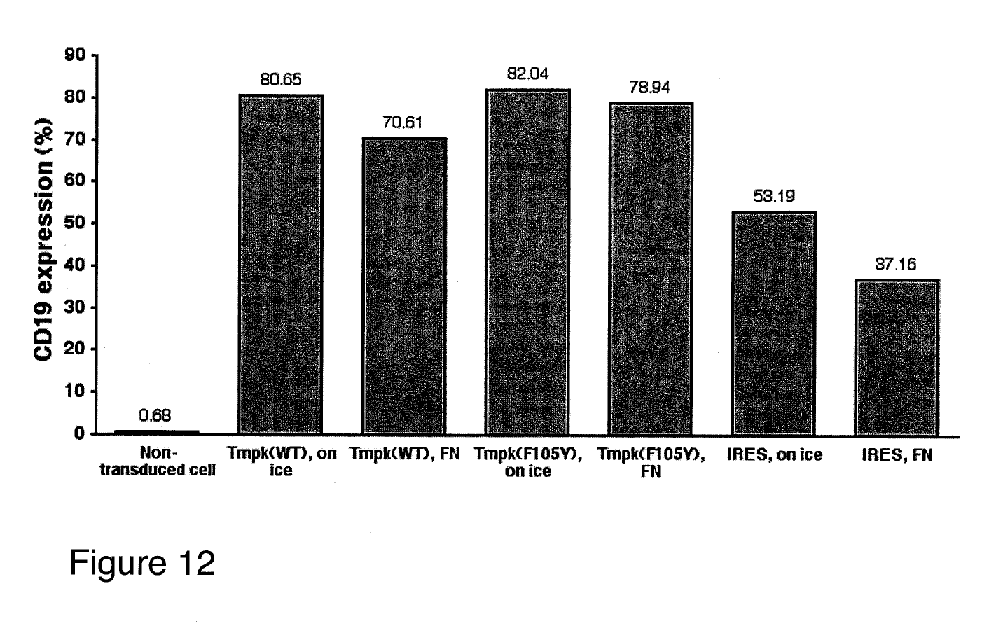

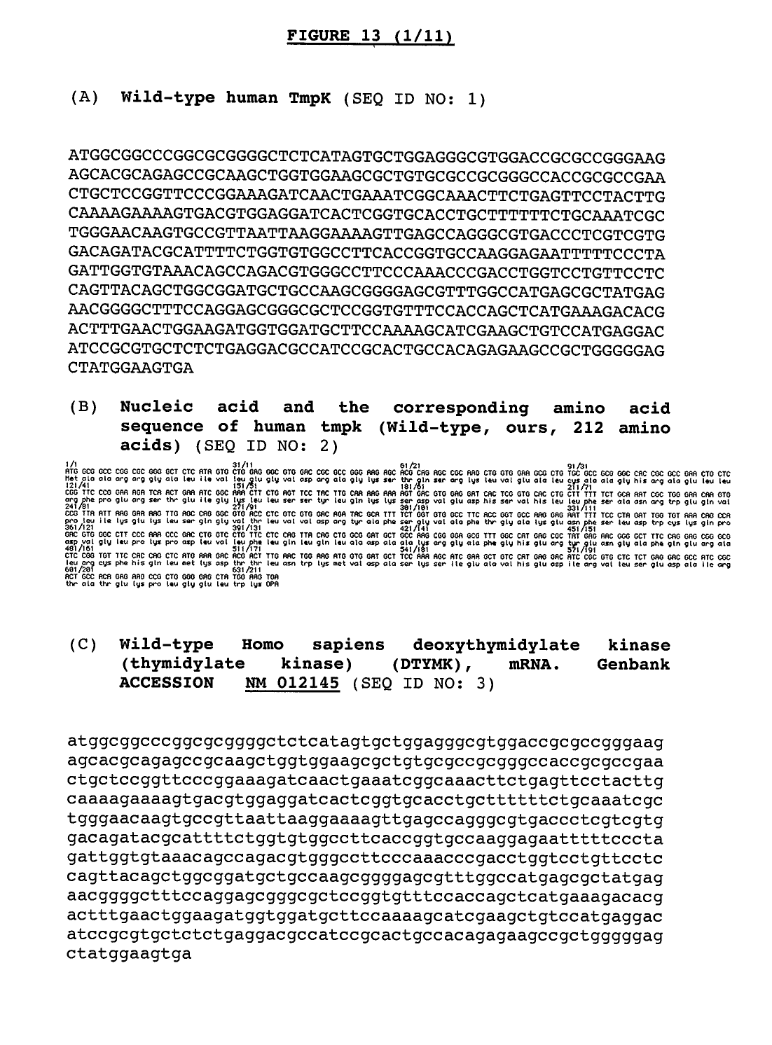

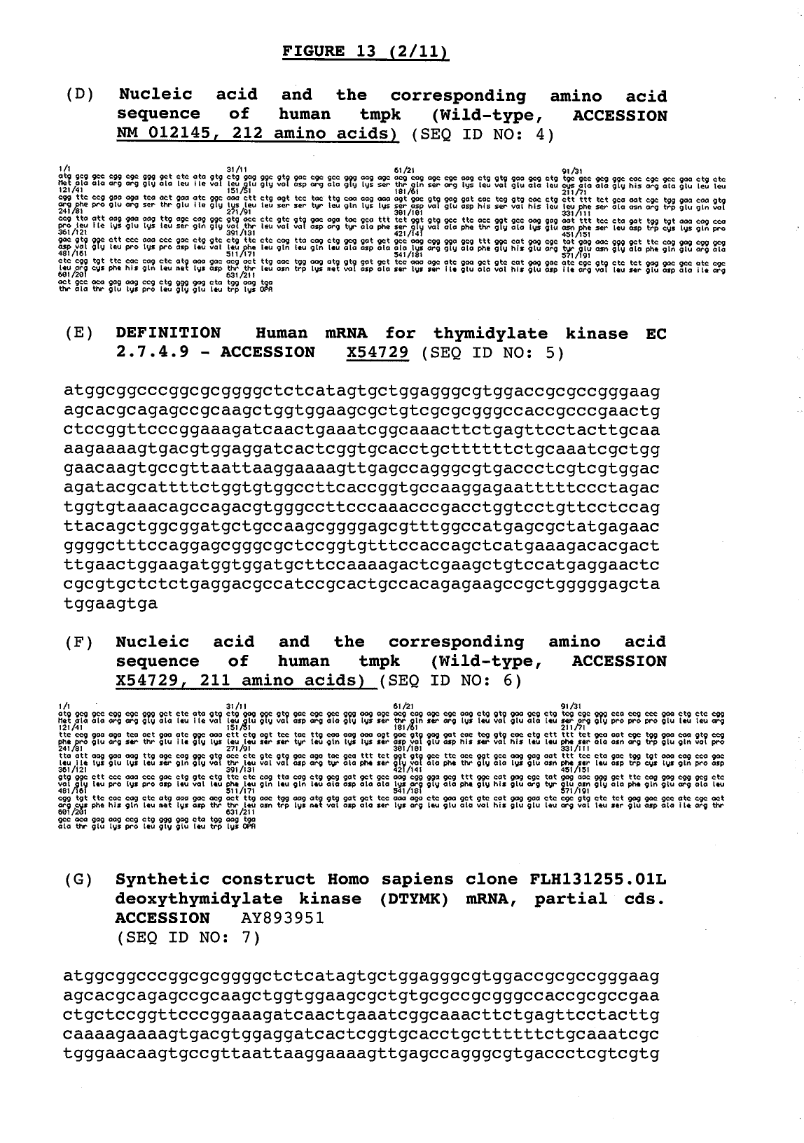

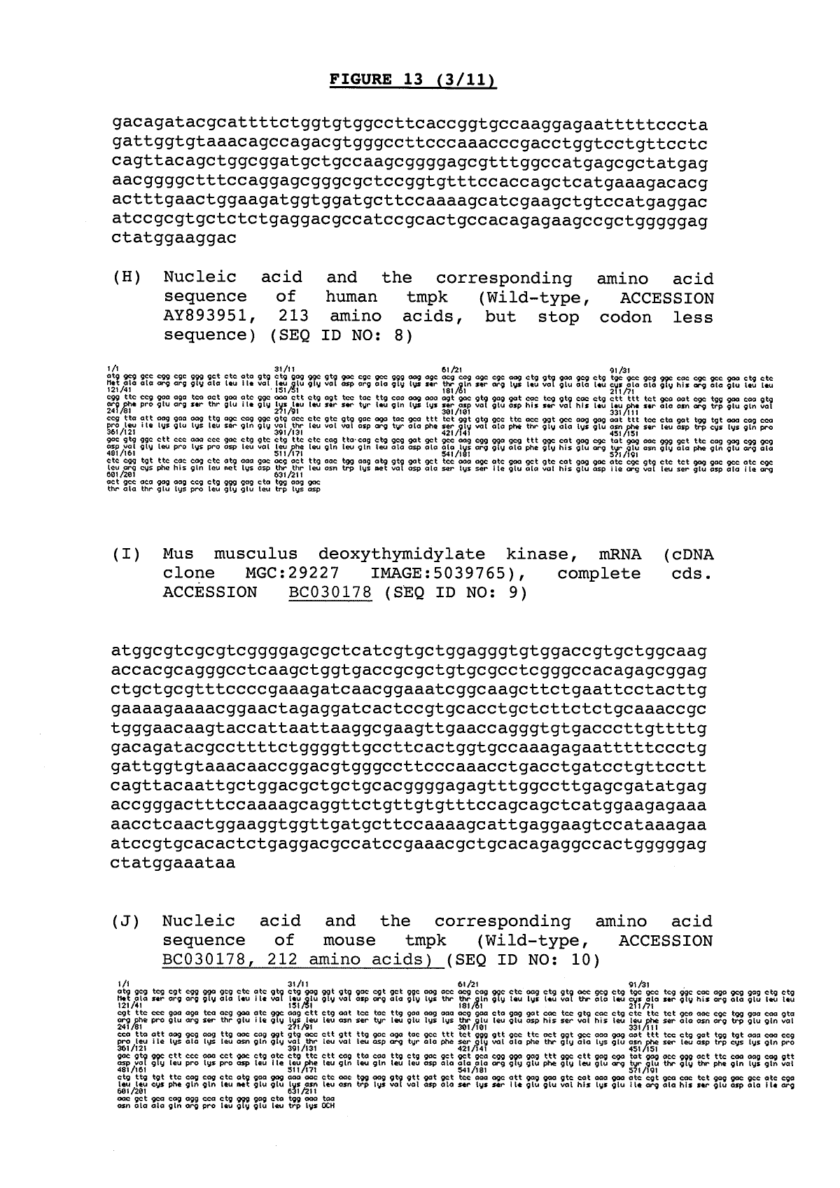

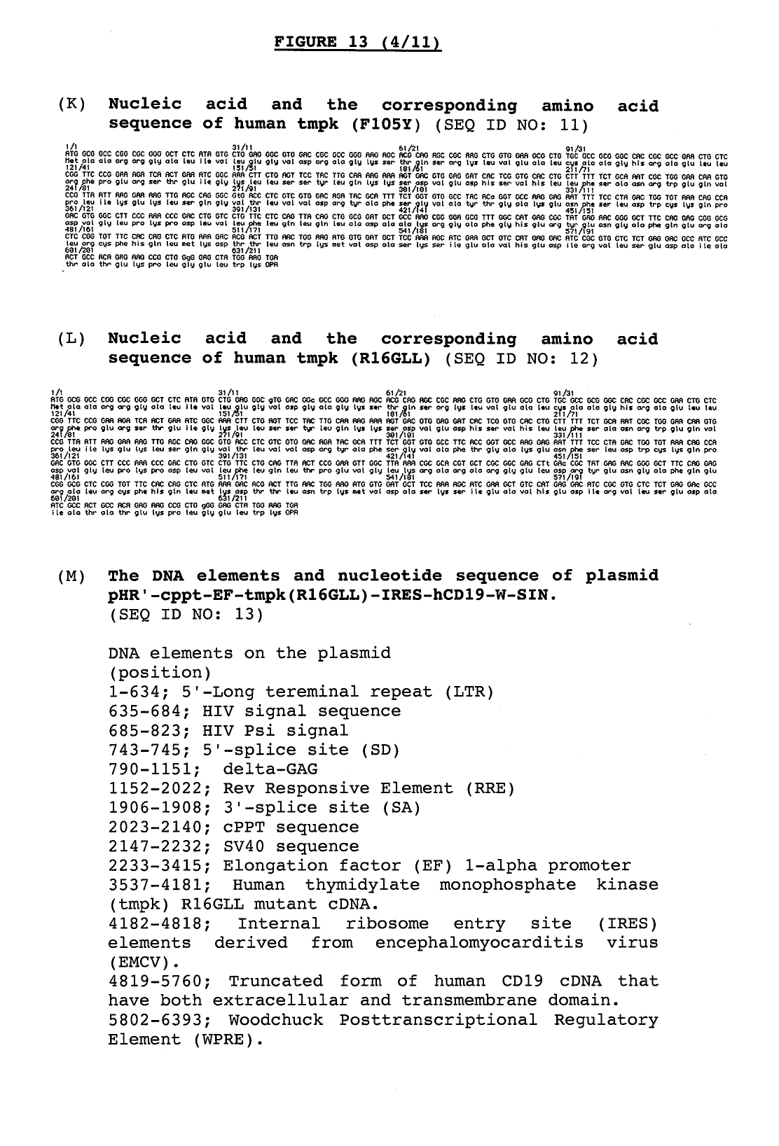

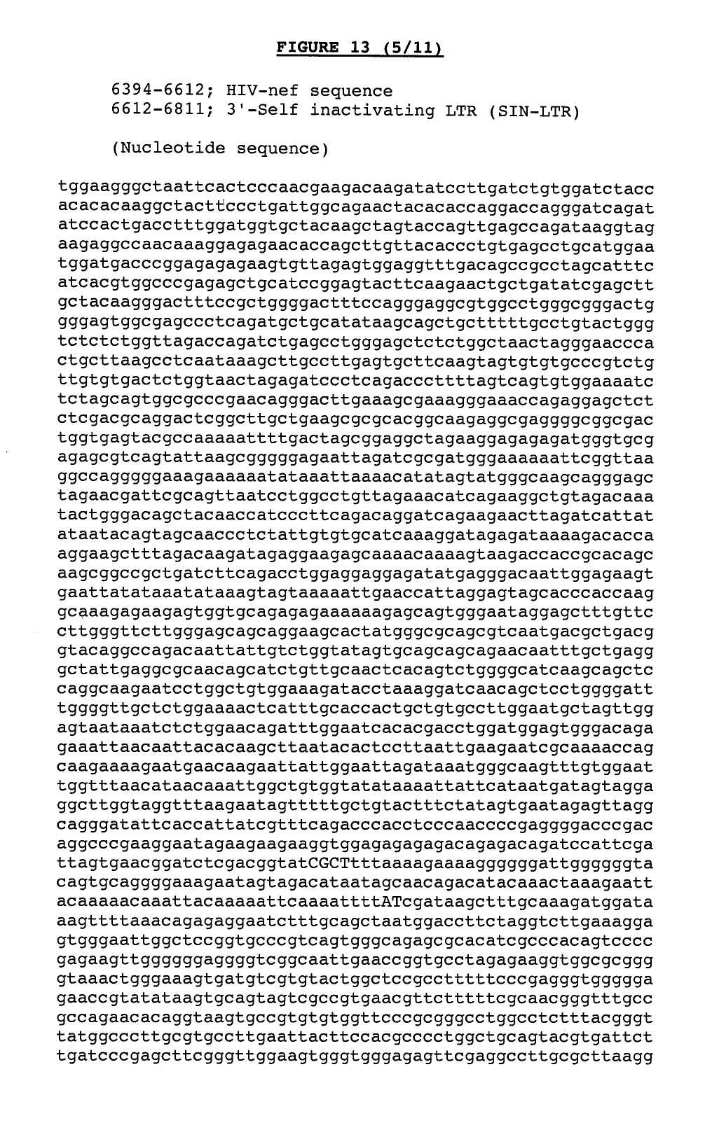

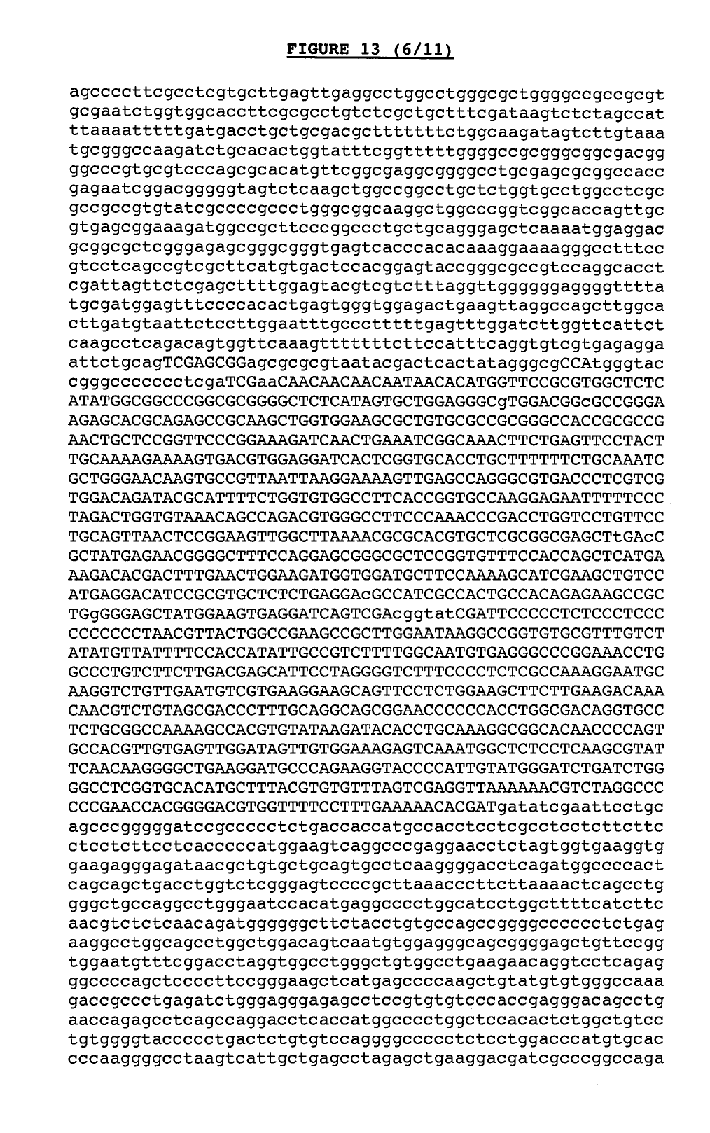

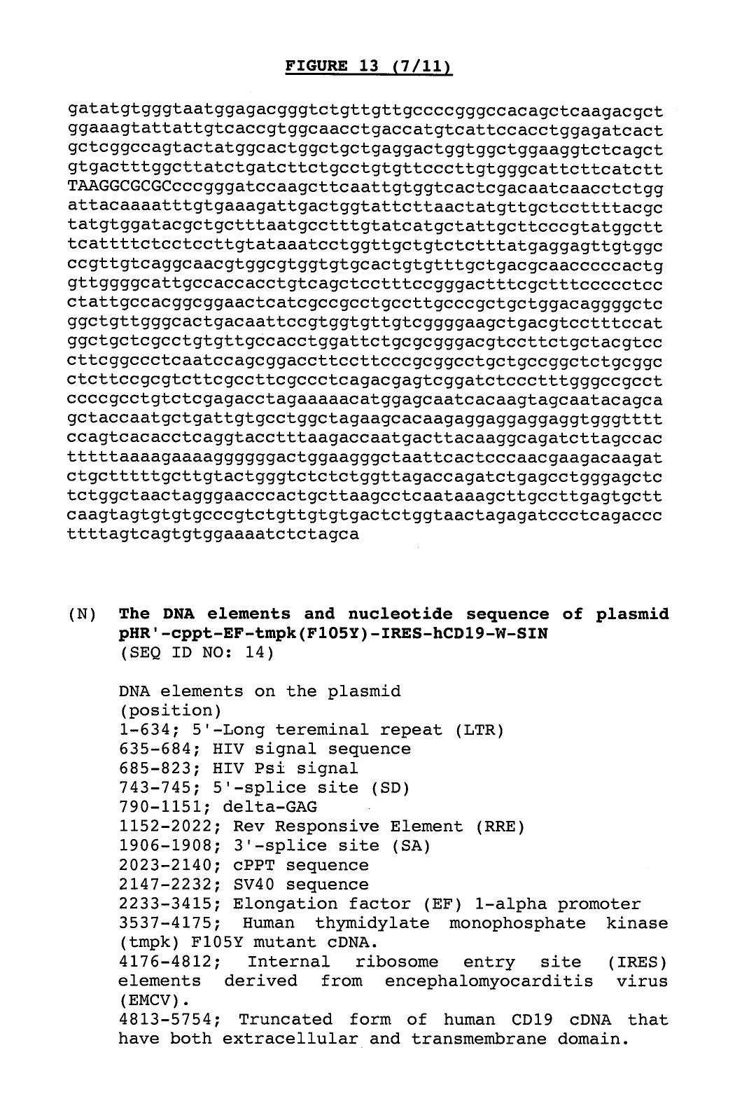

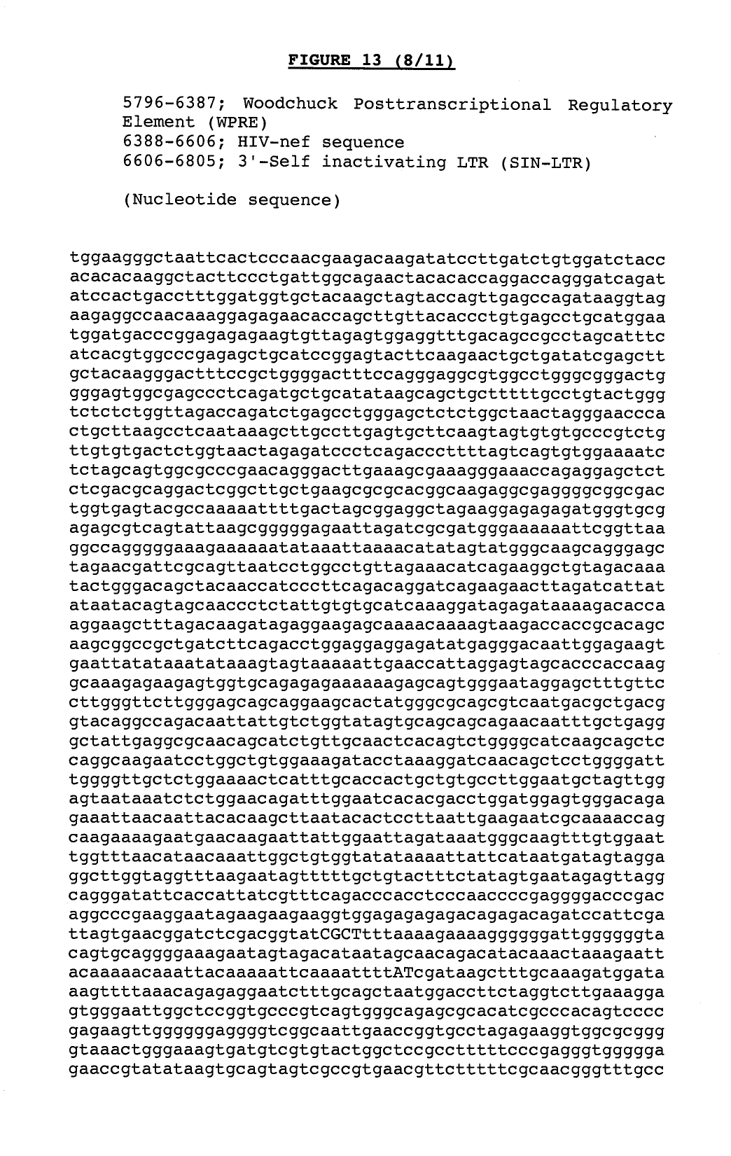

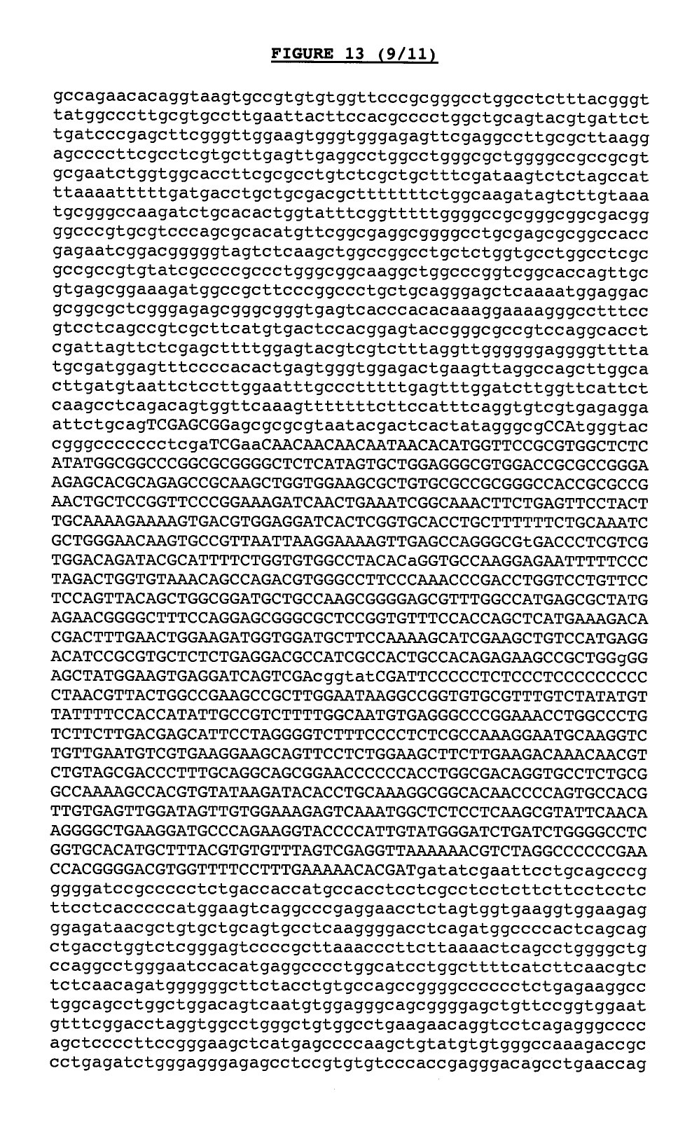

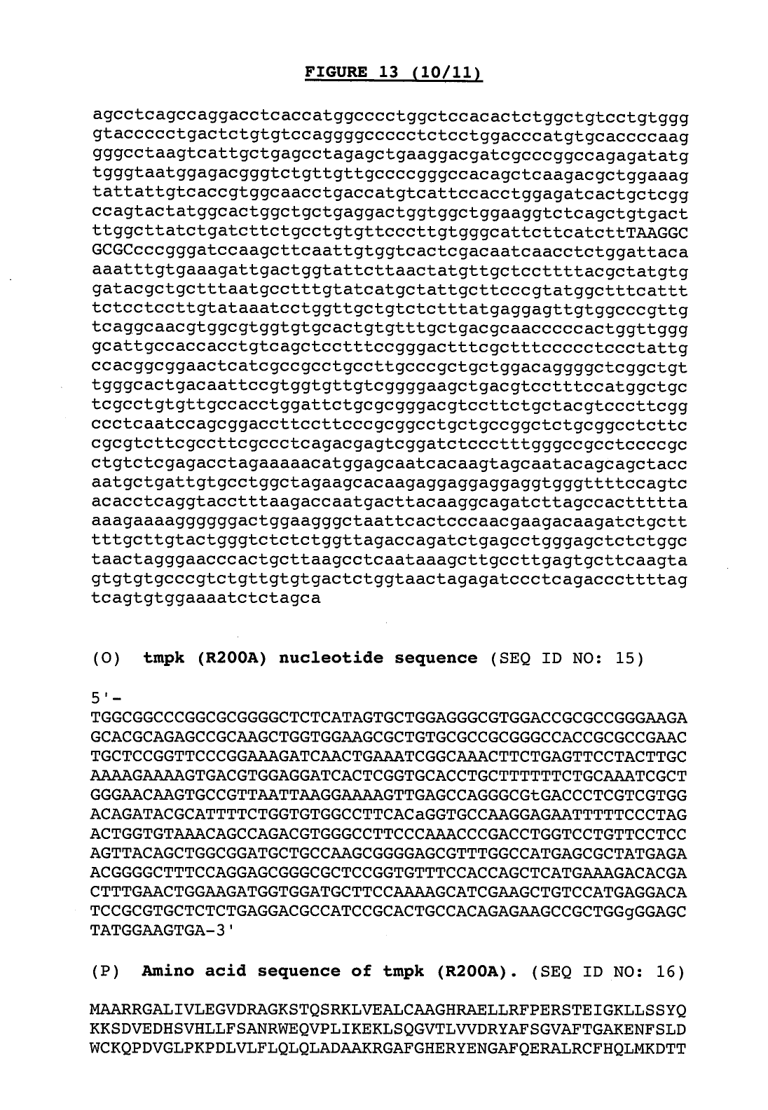

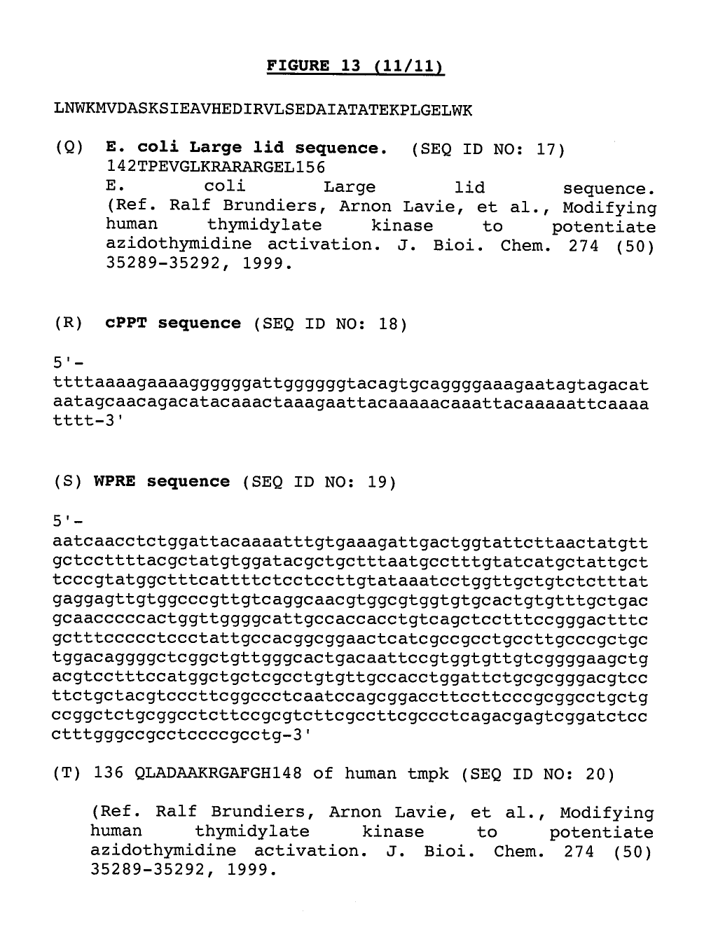

Primary cultures of human and mouse T cells were transduced with LV constructs containing tmpk cDNAs using an MOI as indicated in FIGS. 10-12. After 6 days of culture, T cells were assessed for their level of EGFP or CD19 expression. While no EGFP or CD19 expression was observed in non-transduced cells, strong EGFP or CD19-expression was detected in each of the LV-transduced cell cultures (FIGS. 10, 11 and 12).

### Example 10

The inventors constructed a LV expression system was constructed carrying wild-type or one of two modified forms of tmpk. These engineered tmpk mutants (F105Y and LL) show substantially increased catalytic conversion of AZT compared to wild-type tmpk. Our vector also includes a truncated form of human CD19 (hCD19D), not normally expressed on the T cell lineage, that can be used to enrich and track transduced cells. Highly efficient (95%) transduction of Jurkat cells (human T cell leukemia line) was attained by a single infection with our LVs (MOI of 10). Both LV-tmpk (F105Y)-IRES-hCD19 and LV-tmpk (LL)-IRES-hCD19 transduced cells were efficiently and selectively killed in a dose-dependent manner by AZT (IC₅₀ of 2 μM), while wild-type tmpk transduced cells were unaffected by AZT up to 100 μM. In response to AZT treatment, the apoptotic cell indices of cells transduced with wild-type tmpk, F105Y, or LL were 6.2±0.3%, 40.7±1.7%, and 46.1±4.6%, respectively (n=3). The inventors next established by HPLC that cells transduced with a LV encoding a mutant form of tmpk effectively convert AZT into its active anti-metabolite form, AZT-triphosphate (AZT-TP). Intracellular ratio of AZT-TP to AZT-monophosphate (MP) is 11.3 in cells transduced with a LV encoding the LL mutant of tmpk, compared to 0.02 in non-transduced cells and 0.10 in wild-type tmpk transduced cells. Our findings also revealed that following incubation with indirubin-3-oxime, which inhibits cellular proliferation, and AZT treatment, transduced cells were successfully killed. Thus the cytotoxic mechanism differs from HSV1-tk mediated cell killing and is independent of cell proliferation. The inventors also succeeded in the infection of primary mouse and human T cells to over 40% and 70% transduction efficiency, respectively. Lastly, the inventors have shown that in vivo growth of tumor cells transduced with these mutant tmpk LVs was totally inhibited by treatment with AZT. These results demonstrate that our novel suicide gene therapy system has significant potential for many clinical applications.

### Example 11

### Safety Component of Vectors Used in Gene Therapy

A lentiviral-alpha galactosidase-A GLA)-IRES-tmpk (F105Y) mutant construct is used to transduce the murine myeloid leukemia cell line, C1498. After transduction of the cells with this virus, the congenic recipient GLA-deficient mice will receive the cells by iv-injection. Without prodrug treatment, the host mouse leads to reproducible death from leukemia in a dose-dependence fashion. The host mouse is administered a prodrug. such as AZT. Prodrug treatment results in killing of the responder cells. The enzymatic activity of GLA in the peripheral blood is monitored. The expansion of C1498 cells in the peripheral blood, bone marrow, liver, and spleen of host animals is determined by flow cytometric analysis. Cells are stained for a marker that identifies C1498 cells and not host cells, such as Ly5.1 and for a marker that identifies recipient cells and C1498 cells such as Ly5.2. The survival of mice with or without prodrug-treatment is determined.

### Example 12

### In Vivo GvHD in Mouse Models

Differentially labeled activated T cells are transplanted into permissive murine hosts. Upon determination of GvHD AZT or other nucleoside analogy is administered. The mouse receiving modified tmpk expressing cells exhibits a reduction of GvHD compared to controls. GvHD is eradicated in the mouse.

Ly5.1-mouse derived T cells and/or Ly5.2-mouse derived T cells will be transduced with LV-tmpk (F105Y)-IRES-hCD19 or LV-IRES-hCD19 as well as LV-EGFP as a control using an MOI of 20.

Host mice, CB6F1 will receive total body irradiation with a single dose of lethal irradiation (11 Gy), and transduced cells with T cell depleted bone-marrow cells prepared from CB6F1 recipient mice will be infused into host recipients (20M cells/mouse, n=10 of each group). Mice will be monitored for clinical GvHD everyday.

The following signs are included into clinical index: weight loss, hunching, activity, fur texture, and skin integrity.

T cell chimerism are determined by flow cytometry after bleeding from the tail vein. Plasma is isolated from the remaining blood and stored at −80° C. for later determination of cytokines.

When chimerism of Ly5.1-derived T cells will go up to over 10%, mice will receive daily ip AZT-injections using a dose of 2.5 mg/kg.

Organs will be isolated and prepared for histology and immunohistochemistry to evaluate the T cell infiltration in the tissues.

### Example 13

### Adoptive Transfer of Human T Cells

Activated human T cells are transduced with either a modified tmpk molecule or a control gene. Isolated cells expressing the modified tmpk or control gene are adoptively transferred into permissive murine strains that can accept human xenografts. AZT or other thymidine analog is administered systemically. The number of T cells are determined at various time points to look for evidence of specific killing.

Human Th1 T cell will be transduced with LV-tmpk (F105Y)-IRES-hCD19 or LV-IRES-hCD19 as well as LV-EGFP as a control using an MOI of 20.

Host mice will receive total body irradiation with a single dose of lethal and transduced cells will be infused into host recipients (20M cells/mouse, n=10 of each group). Mice will be monitored for clinical GvHD everyday. The following signs are included into clinical index: weight loss, hunching, activity, fur texture, and skin integrity.

Human chimerism are determined by flow cytometry after bleeding from the tail vein. Human chimerism is calculated as follows: human chimerism (%)=[huCD3+/(huCD3++mCD45+)]×100. Plasma is isolated from the remaining blood and stored at −80° C. for later determination of human IgGs and cytokines.

When human chimerism will go up to over 10%, mice will receive daily ip AZT-injections using a dose of 2.5 mg/kg.

Organs will be isolated and prepared for histology and immunohistochemistry to evaluate the T cell infiltration in the tissues.

### Example 14

### Bystander Killing Effects

PC3 cells are transduced using LV-tmpk (wild-type)-IRES-hCD19 or LV-tmpk (F105Y)-IRES-hCD19 and tmpk-overexpressing cells are screened by Western blotting using rabbit anti-human tmpk antibody. The resultant cells are used for checking the AZT-sensitivity. The cells are split into 96-well plates (2500 cells/well), and expose to AZT for 4 days. Cell viabilty is determined using MTS-reagent. For bystander studies, the tmpk-transduced cells are cocultured with LV-EGFP transduced PC3 cells in 24 well plate (50000 cells/well). After incubation with 100 μM AZT for 4 days cells, the percentage of EGFP-positive cell in each wells are determined by flow cytometry. If the bystander cell killing occur, EGFP-positive cell population treated with AZT show the decrease in their number compared to that without AZT-treatment.

### Example 15

### Materials and Methods

cDNA Cloning of Human CD19 and Construction of LV Shuttle Vector

Total RNA was extracted from the human Burkitt's lymphoma cell line (Raji) using the TRIZOL reagent (Invitrogen, Carlsbad, Calif.). cDNA templates were generated from total RNA by reverse transcription using oligo-dT primer and Superscript II reverse transcriptase (Invitrogen). The cDNA of full-length huCD19 was obtained by PCR using Platinum Hifi Taq DNA polymerase (Invitrogen) and primers CD19 F1 and CD19 R1 described below. The amplified PCR product was directly ligated into the TA-vector, pPCR-script SK (+) (Stratagene, La Jolla, Calif.) to give pPCR-huCD19full. A truncated form of huCD19 (huCD19Δ), which has the extracellular and transmembrane domains but lacks the cytoplasmic domain, was generated by inverse PCR from pPCR-huCD19full using primers CD19 F2 and CD19 R2 (described below), to give pPCR-huCD19Δ. The F2 primer has a complementary sequence to the stop codon just after the end of the transmembrane domain. Following sequence confirmation of the cDNA inserts in pPCR-huCD19Δ, the cDNA fragments were then isolated and subcloned into the EcoRI site of the shuttle vector pSV-IRES that has a sequence for an IRES element from the EMCV, to give pSV-IRES-huCD19Δ. The primer sequences used for subcloning of the human CD19 cDNA were as follows: CD19 F1: 5′-atgccacctcctcgcctcctcttcttcc-3′ and CD19 R1: 5′-tcacctggtgctccaggtgccc-3′. The truncated CD19 construct was made by inverse-PCR using primers CD19 F2: 5′-ccgccaccgcggtggagctccag-3′ and CD19 R2: 5′-ttaaagatgaagaatgcccacaaggg-3′.

Subcloning of Human Tmpk cDNA and Construction of Bicistronic LVs

To subclone the cDNA for wild-type (WT) human tmpk, PBMNCs were isolated from heparinized blood obtained from healthy donors by Ficoll-Hypaque density gradient separations (GE Healthcare Biosciences, Inc. Freiburg, Germany). The WT human tmpk cDNA was amplified by PCR using first strand cDNA generated from PBMNC RNA by the method above. PCR products containing the WT tmpk cDNA were subcloned into pPCR-scriptSK (+) and sequenced. Mutant forms of tmpk, denoted F105Y and R16GLL, were previously generated^{23,24}. The cDNAs for the WT and each mutant form of tmpk were first subcloned into a shuttle vector (pSV-IRES-huCD19Δ) to construct bicistronic expression cassettes that allow simultaneous expression a single mRNA strand, encoding the suicide gene and huCD19Δ. The constructs were then each subcloned downstream of the internal EF1a promoter into an HIV-1-based recombinant LV plasmid, pHR′-cPPT-EF-W-SIN²⁷. As a control for the transduction experiments, the inventors constructed a pHR′-cPPT-EF-IRES-huCD19Δ-W-SIN LV by subcloning the IRES-huCD19Δ cassette from the pSV-IRES-huCD19Δ plasmid into pHR′-cPPT-EF-W-SIN. In addition, the inventorsused the pHR′-cPPT-EF-enGFP-W-SIN LV³² containing the enhanced GFP (enGFP AKA EGFP) cDNA.

### Preparation of High-Titer LV.

Vesicular stomatitis virus glycoprotein (VSV-g)-pseudotyped lentivectors (LVs), including an enGFP marking vector, were generated by transient transfection of 293T cells with a three plasmid system (the aforementioned pHR′ plasmid constructs, the packaging plasmid pCMVΔR8.91, and the VSV-g envelope encoding plasmid pMD.G³² using CaPO₄ precipitation. Viral supernatants were harvested 48 h later, passed through a 0.45 μm filter, and suspended in PBS containing 0.1% (w/v) BSA after ultracentrifugation at 50,000×g for 2 h at 4° C. The concentrated viral supernatants were serially diluted and titered on 293T cells. Transgene expression in transduced cells was assessed 72 h later using a FACS Calibur (BD Biosciences, San Jose, Calif.) following staining of the transduced and control cells with monoclonal mouse anti-human CD19 conjugated with PE (BD Biosciences) or for enGFP expression. Analysis of the data was performed using Cell Quest software (BD Biosciences).

### Transduction and Analysis of Transgene Expression by Flow Cytometric Analysis.

Cells of the human T lymphoma cell line, Jurkat, and of the human erythro-leukemic cell line, K562, were maintained in RPMI 1640 supplemented with 10% FBS (CPAA Laboratories, Etobicoke, ON), 100 U/ml of penicillin, and streptomycin to 100 μg/ml (both Sigma, Oakville, ON). Cells were infected with concentrated virus stocks using an MOI of 10 in the presence of 8 μg/ml protamine sulfate. Infected cells were then kept in culture for 5 days prior to evaluating gene transfer efficiency. Gene transfer efficiencies were measured by flow cytometry as described above. Individual clone cell lines were used for all subsequent experiments. They were derived by limiting dilution and selected based on comparable huCD19Δ expression as determined by flow cytometry (above).

To compare the relative expression levels of tmpk, the transduced cells were first fixed with 4% buffered formalin for 15 min then permeabilized by treatment with PBS containing 0.1% Triton X-100 for 10 min. Cells were incubated with 20% normal goat serum for 30 min and then incubated with rabbit anti-human tmpk (diluted 1:500) for 1 h. The cells were further incubated with goat anti-rabbit IgG conjugated to Alexa488 (diluted 1:500, Molecular Probes Inc., Eugene, Oreg.) for 1 h. All incubations were performed at room temperature. Levels of tmpk expressed in the transduced cells were determined by flow cytometry.

### HPLC for AZT-Metabolites.

Cells were cultured in the presence of 100 μM AZT for 36 h. 10⁷ cells were homogenized by sonication in 100 μl of 5% (w/v) trichloroacetic acid (TCA). The supernatant was collected after centrifugation at 10,000×g for 15 min at 4° C. TCA was removed by extraction with an equal volume of 20% tri-n-octylamine in pentane. The neutralized aqueous fraction was directly injected into the HPLC machine (Waters, Milford. MA). Separation of AZT and its metabolites were performed on a C18 column (Waters), with a mobile phase composed of 0.2 M phosphate buffer containing 4 mM tetrabutylammonium hydrogen sulfate (pH 7.5) and acetonitrile in the ratio of 97:3 (v/v)⁴⁸ at a flow rate of 1.5 ml/min. The UV absorbance was monitored at 270 nm. Standards for each AZT-metabolite (AZT-MP, AZT-DP, and AZT-TP) were purchased from Moravek Biochemicals (Brea, Calif.). Five million cell equivalents were injected and analyzed in triplicate.

### Determination of AZT-Sensitivity of Tmpk-Transduced Jurkat Cells.

Transduced Jurkat cells and single-cell clones were seeded in 96 well plates (2×10⁵ cells/well) in 200 μl of the RPMI medium described above with increasing concentrations of AZT (0, 0.1, 1, 10, 100 μM, and 1 mM). The medium was changed daily. After 4 days of culture, cell viability was determined by using Cell Titer 96 Aqueous One Solution Cell Proliferation Assay kit (Promega, Madison, Wis.).

For evaluation of the induction of apoptosis, treated Jurkat clonal cells were stained with Annexin V. Briefly, cells were seeded in 24 well plates (10⁶ cells/well) in 1 ml of medium with or without 100 μM AZT. After 4 days of culture, Annexin V staining was performed according to the manufacturer's protocol (Annexin V-APC: BD Pharmingen). For testing whether AZT-mediated cell killing depends on the cellular proliferation, indirubin-3′-monoxime (final concentration 5 μM, Sigma-Aldrich, St. Louis, Mo.) was added simultaneously with 100 μM AZT to the culture.

To simplify comparative studies a relative apoptotic index was calculated. Here data obtained was normalized by dividing results from AZT treated cells in each condition by the results obtained without added AZT. Values were reported as fold increases. Statistical significance between groups was calculated by ANOVA.

Transduction of Primary T Cells with LVs and Evaluation of Induction of Apoptosis Following AZT Exposure

Human T lymphocytes were isolated from PBMNCs contained within heparinized blood obtained from healthy human donors by Ficoll-Hypaque (GE Healthcare) separations. Mouse T cells were prepared from B cell-depleted splenocyte preparations using goat anti-mouse IgG beads (BioMag, Qiagen, Mississauga, ON). T cells were activated by using anti-CD3 and anti-CD28 coated beads (PMID: 12855580) in a ratio of 1:3 (cell:beads) with 20 IU/mL of recombinant human interleukin 2 (R&D Systems, Minneapolis, Minn.) for 3 days. Cells were infected with concentrated virus stocks for 3 h on ice using an indicated MOI in the presence of 8 μg/ml protamine sulfate. Infected cells were then kept in culture for 5 days prior to evaluating gene transfer efficiency. Gene transfer efficiencies were measured by flow cytometry using a monoclonal anti-human CD19-antibody conjugated with phycoerythrin (PE) as described above. Induction of apoptosis following AZT-exposure was evaluated by Annexin V-staining as above.

### Measurement of Mitochondrial Inner Membrane Potential and Activation of Caspase-3.

Transduced cells (106) were treated with 100 μM AZT for 4 days or left untreated. To detect changes in the mitochondrial inner membrane potential, the cells were incubated with 5,5′,6,6′-tetrachloro-1,1′,3,3′-tetraethylbenzimidazolylcarbocyanine iodide (JC-1, Molecular Probes Inc.) for 30 min at 37° C., and were then analyzed using a FACS Calibur. The activation of caspase-3 in cells was examined using the FACS Calibur following incubation with an FITC-labeled caspase 3 inhibitor peptide (FITC-DEVD-FMK, Calbiochem, San Diego, Calif.) for 1 hr at 37° C.

### Transduced K562 Cells in a NOD/SCID Xenograft Model

Transduced K562 cells were affinity-purified by MACS using magnetic beads conjugated with an anti-human CD19 monoclonal antibody (Miltenyi Biotec Inc., Auburn, Calif.). The purity of the cells following isolation was evaluated by FACS Calibur. Non-obese diabetic/severe combined immunodeficiency (NOD/SCID) mice (5 to 8-weeks old, purchased from Jackson Laboratories, Bar Harbor, Me.) were maintained at the Animal Resource Centre at the Princess Margaret Hospital (Toronto, ON, Canada). The entire animal experimental procedure followed a protocol approved by the Animal Care Committee of the UHN. Experimental groups consisted of male and female NOD/SCID mice injected with 2×10⁷ K562 cells (resuspended in 0.5 mL D-PBS; Oxoid, Basingstoke, England) that were either lentivirally-transduced (n=10 for each LV) or non-transduced (NT) (n=10). Injections were performed subcutaneously (sc) into the dorsal right flanks of recipient mice as previously described⁴⁹. One day after injection of the cells, half of the mice in each group (n=5) began receiving daily AZT injections, administered intraperitoneally (ip) at a dose of 2.5 mg/kg/day for 14 days. Tumor growth was measured by caliper and calculated as 0.5×length×width² (in mm³) for up to 14 days post-inoculations.

### Statistical Analysis

Data are presented as the mean±standard error of the mean (SEM) for in vitro experiments and the mean±standard deviation of the mean (SD) for in vivo experiments. Statistical analyses were performed using StatView version 4.5 software for Macintosh (SAS). For in vitro experiments, a one-way analysis of variance (ANOVA) with either a Bonferroni or a Dunnett post-hoc test was used to determine statistically significant results with the level of significance set at P<0.05. Statistical comparison of means was performed by a two-tailed unpaired Student's t test for in vivo experiments.

### Results

### Synthesis of Novel Suicide LVs Expressing Modified Tmpks and Truncated CD19

FIG. 1 shows a schematic structure of the LVs constructed for this study. Jurkat cells were transduced a single time with our recombinant LVs using an MOI of 10. Five days after transduction, CD19 expression on transduced cells was examined. While no CD19 expression was observed on non-transduced (NT) Jurkat cells, over 95% of the cells transduced with each LV showed strong CD19-expression (data not shown). Next, individual cell clones were isolated by flow cytometry and limiting dilution. The mean fluorescent intensity (MFI) of huCD19Δ expressed on isolated clones of cells transduced with each LV showed similar values (data not shown). To compare the expression levels of the upstream tmpk gene on a gross level, transduced cells were also examined by flow cytometry following intracellular immunostaining with rabbit anti-human tmpk. Since tmpk is endogenously expressed in Jurkat cells, the inventors detected basal expression of tmpk in NT cells. Cells transduced with LV-tmpk(WT)-IRES-huCD19Δ or either LV-tmpk mutant-IRES-huCD19Δ showed an increase in tmpk expression, up to 5 times higher compared with non-transduced and LV-IRES-huCD19 transduced cells (data not shown).

### Determination of the Major Intracellular AZT Metabolites in LV/Tmpk-Transduced Cells

To confirm functionality of the tmpk mutants overexpressed in transduced cells for the metabolic conversion of AZT, the intracellular amounts of AZT-metabolites were measured by reverse-phase HPLC. Following a 36 h incubation with 100 μM AZT, the cells expressing the R16GLL mutant tmpk efficiently converted AZT-MP into AZT-DP and then to the cytotoxically active metabolite form, AZT-TP, whereas the main metabolite that accumulated in the NT-Jurkat cells was AZT-MP (FIG. 2A). Also no significant increases in the accumulation of AZT-TP or induction of cell death in the cells overexpressing WT tmpk itself were observed (data not shown). To compare the effectiveness of the conversion of AZT-MP to its active metabolite, AZT-TP, the ratio of AZT-TP to AZT-MP in each cell population was calculated from the values of the area under curve of each chromatogram. FIG. 6B shows that overexpression of the R16GLL mutant induced a 615-fold increase (P<0.0001) in the AZT-TP/AZT-MP ratio compared to that of the NT cells, the tmpk WT-overexpressing cells, or the LV-IRES-huCD19Δ-transduced cells. SIMILAR DATA WAS ALSO OBTAINED FOR THE F105Y MUTANT. These data indicate that the cells overexpressing this mutant form of tmpk more efficiently converted AZT-MP to AZT-DP, which was subsequently transformed into its active antimetabolite, AZT-TP, likely by cellular nucleotide diphosphate kinase¹².

### AZT Sensitivity of Tmpk-Transduced Cells

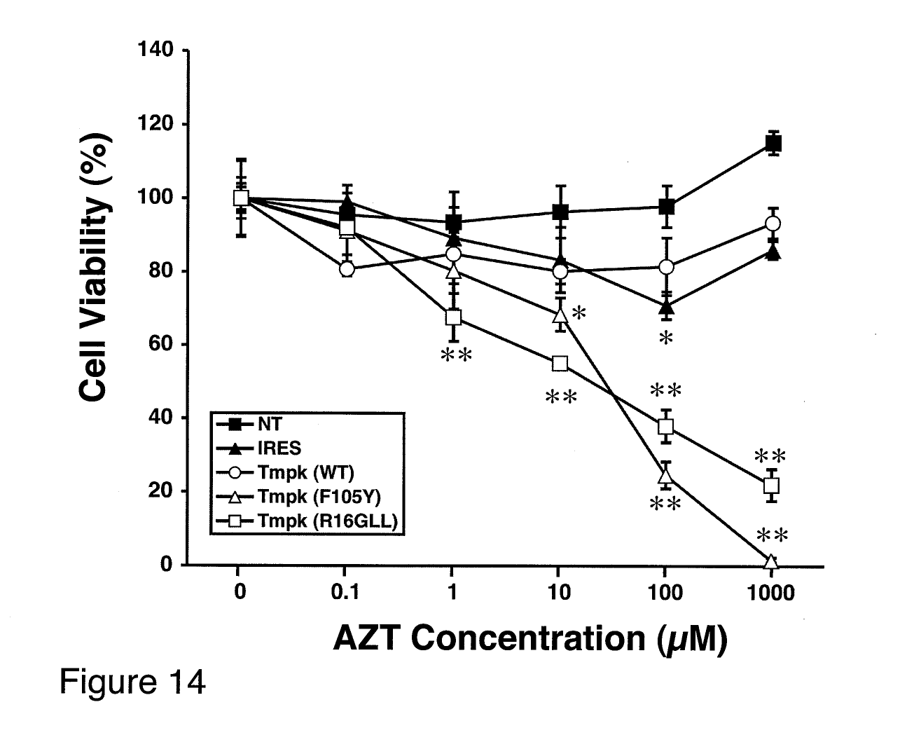

As transduced cells expressing the mutant forms of tmpk revealed differences in intracellular accumulation of AZT-metabolites, the effect of exposure to AZT on cell viability was next measured. Note that by itself, transduction of Jurkat cells with LVs engineering expression of controls or our modified suicide genes and huCD19Δ did not affect their proliferation (data not shown). For the sensitivity experiments the tmpk-expressing cells were incubated with increasing concentrations of AZT, and after four days determined the percentage of living cells using an MTT assay (FIG. 14). Transduced cells expressing the tmpk mutants F105Y or R16GLL were minimally viable upon addition of AZT in a dose-dependent manner (P<0.0001). In contrast, limited cell killing, even at high doses of AZT up to 1 mM, was observed in the negative control cells including: the tmpk WT- and LV-IRES-huCD19Δ-transduced cells as well as the NT Jurkat cells (P values for the tmpk WT, LV-IRES-huCD19Δ-transduced, and NT cells were 0.0677, 0.0426, and 0.1375, respectively).

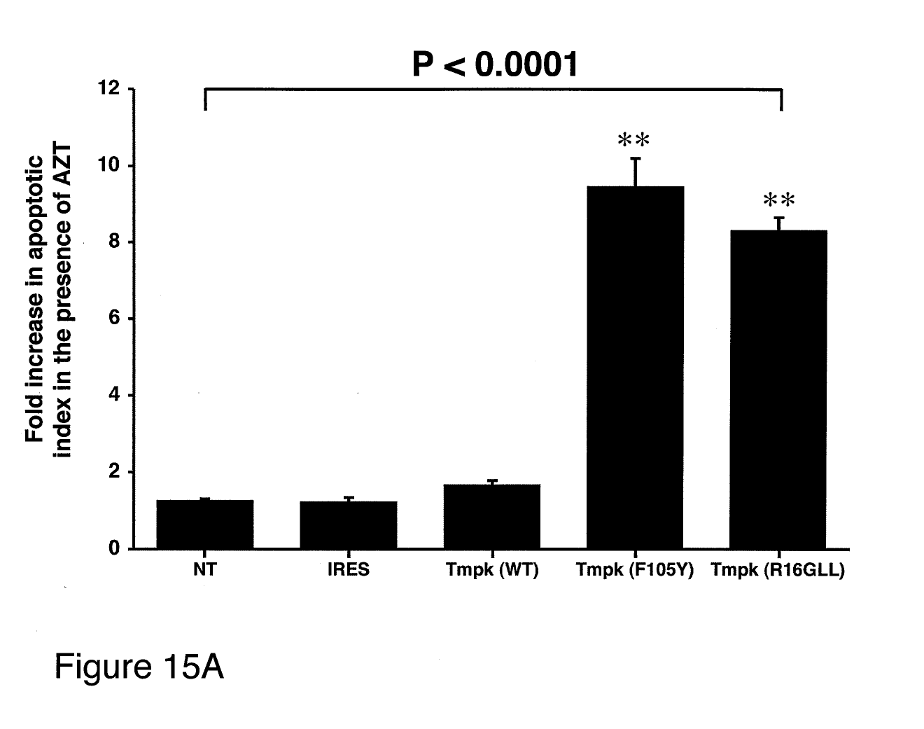

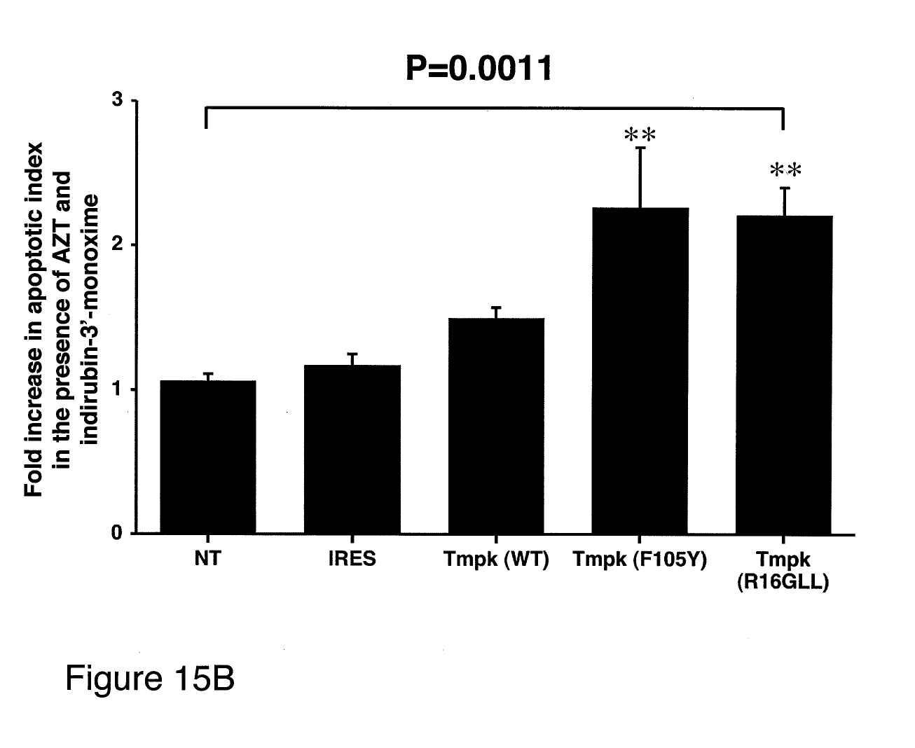

Since the formation of nuclear apoptotic bodies were observed by DAPI-staining in the tmpk-mutant-expressing cells treated with AZT (data not shown), active metabolites of AZT may have induced cellular death by apoptosis. The induction of apoptosis in the tmpk-expressing cells was examined following AZT treatment, by staining the cells with Annexin V and performing flow cytometric analyses. In response to AZT exposure, early apoptotic indices of cells expressing the F105Y and the R16GLL tmpk mutants were significantly increased (FIG. 15A) compared to those in the absence of AZT treatment (9.5±0.8, and 8.3±0.4-fold increases of apoptotic index by AZT-treatment for F105Y- and R16GLL-expressing cells, respectively).

HSV-tk-mediated cell killing has been reported to require cellular proliferation to demonstrate the cytotoxic effect of the produced anti-metabolites through DNA chain termination⁴⁰. Thus, for these experiments, the cytotoxic events of AZT on tmpk-expressing cells were assessed to see if they were also dependent on cellular proliferation. Transduced cells were cultured with or without 100 μM AZT in the presence of indirubin-3′-monoxime to arrest cell cycle progression. After 4 days incubation with 5 μM indirubin-3′-monoxime in the absence of AZT, the cells showed cell cycle arrest at G2/M-phase (data not shown). By treating the cells with 100 μM AZT in the presence of 5 μM indirubin-3′-monoxime, the apoptotic indices of the F105Y- and R16GLL-expressing cells were still significantly increased (FIG. 15B) compared to those without AZT-treatment (2.3±0.4, and 2.2±0.2-fold increases, respectively). No significant increases were seen in the apoptotic indices of NT cells, WT tmpk-overexpressing cells, or control LV-IRES-huCD19Δ-transduced cells (FIG. 15B). This suggests that the induction of apoptosis by AZT in the tmpk mutant-expressing cells is, in part, independent of their proliferation status.

Transduction and AZT Sensitivity of Primary Human and Mouse T Cells

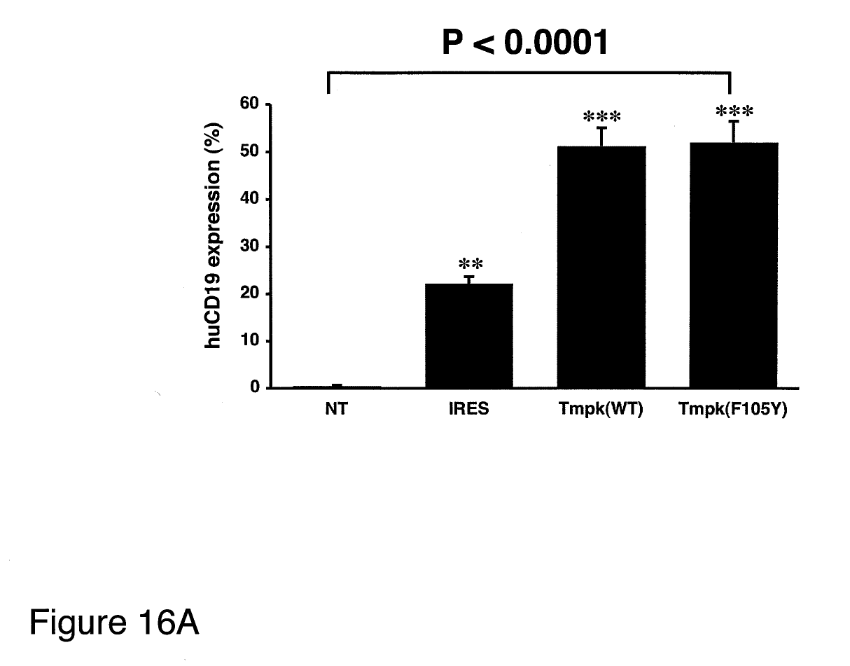

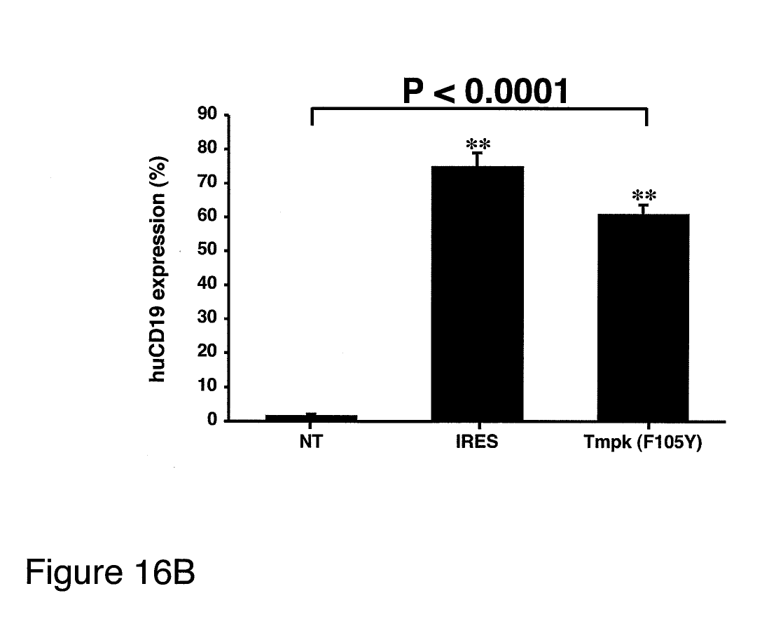

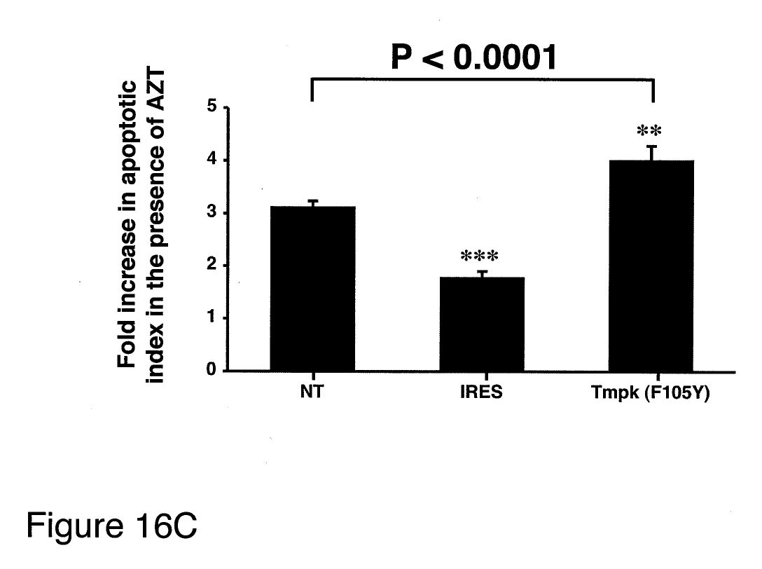

Primary cultures of human and mouse T cells were transduced with LV tmpk constructs using an MOI of 20. The LV-tmpk (R16GLL) mutant was not used for the transduction of primary T cells since this version contains bacterial tmpk-sequence that could cause an eventual immunogenic response when used in vivo. After 6 days of culture, transduced and control T cells were assessed for their level of huCD19 expression. While very low huCD19 expression was observed in NT cells, huCD19 expression on primary mouse T cells was significantly increased in each of the LV-transduced cultures reaching levels of >50% (FIG. 16A). Likewise, even higher levels of huCD19 expression were observed on productively transduced human T cells reaching levels of >60% (FIG. 16B). These levels are considerable given that expression of downstream genes in bicistronic cassettes may be only 10% or less of upstream gene expression PMID: 10933956. To test the AZT sensitivity of the productively transduced human T cells, the cells were exposed to 100 μM AZT for 4 days and induction of apoptosis was measured by Annexin V staining. Although the early apoptotic indices of primary NT human T cells were increased by AZT exposure at this dose, the apoptotic index of cells expressing the F105Y tmpk mutant was significantly increased (FIG. 16C) compared to those without AZT treatment (4.0±0.3-fold increases; P<0.0001).

### Novel Suicide Mechanism Utilized by the Tmpk/AZT Axis

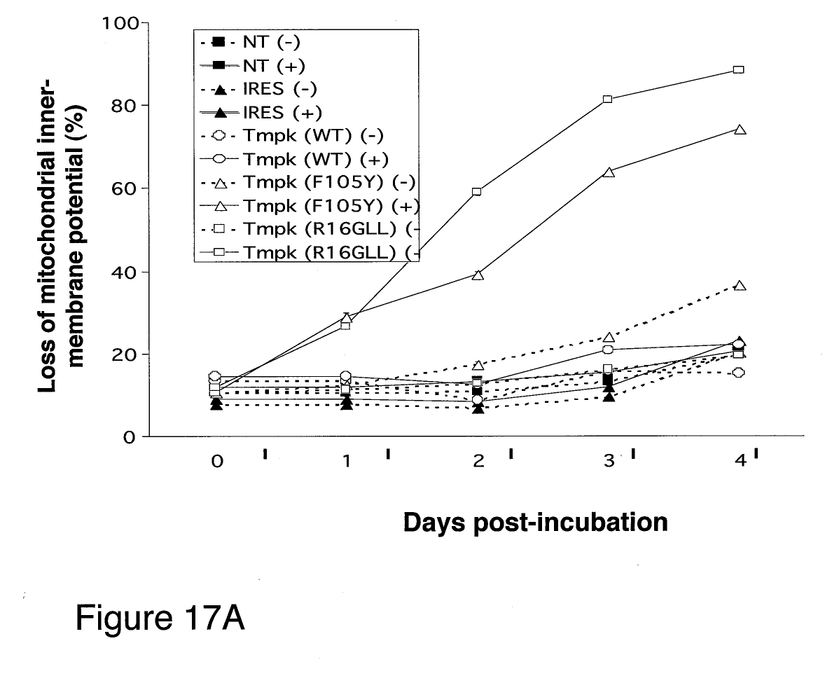

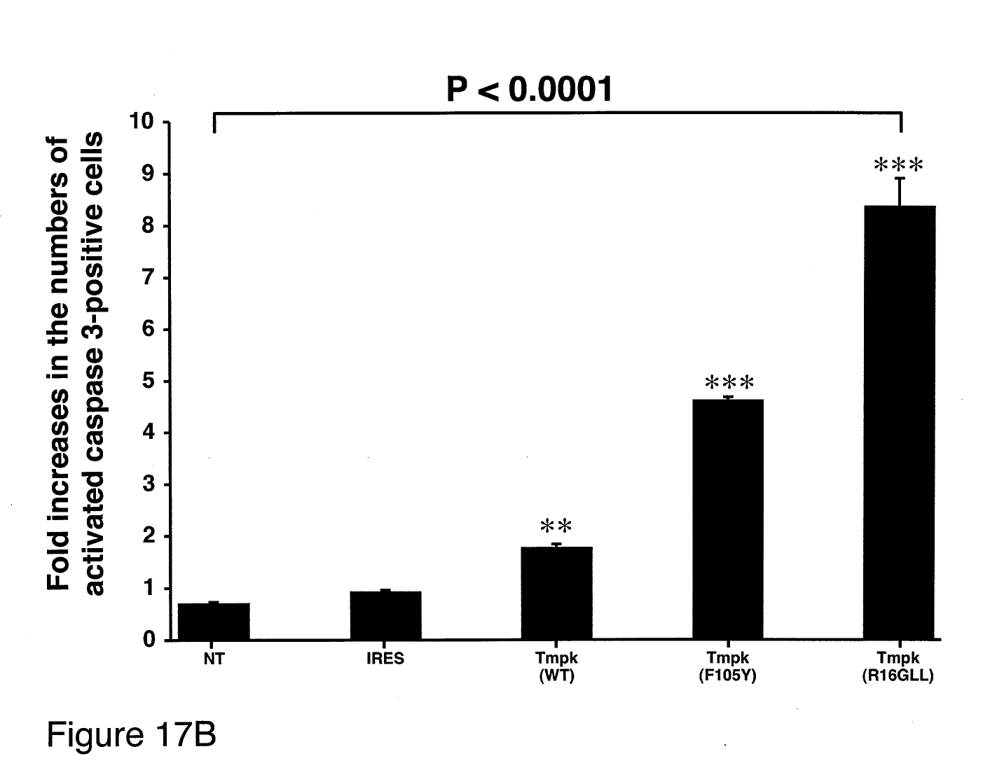

AZT is a potent inhibitor of HIV replication. That said, HIV patients treated with AZT sometimes develop toxic mitochondrial myopathy through induction of mitochondrial biochemical dysfunction^{18,20,21}. In order to decipher the mechanism of cellular apoptosis induction in the tmpk-mutant-expressing cells following AZT treatment, the mitochondrial inner membrane potential was measured in intact cells. This gives a direct indication of the activity of mitochondrial energy metabolism. For these experiments, a fluorescent probe, JC-1, was used to examine living cells by flow cytometry. JC-1 is a dye that emits a green fluorescence at low mitochondrial membrane potential⁴¹. At higher membrane potentials, JC-1 forms red fluorescence-emitting “J-aggregates”. A significant increase (P<0.0001) in the loss of mitochondrial inner membrane potential occurred in both the F105Y- and the R16GLL-expressing Jurkat cells (FIG. 17A) following 4 days of AZT treatment compared to controls. Negative control cell groups including the NT-, the WT-overexpressing, or the LV-IRES-huCD19Δ-transduced cells treated with AZT did not demonstrate a similar loss of mitochondrial inner membrane potential (FIG. 17A).

Caspase 3 is a key molecule in the cellular apoptosis pathway; loss of mitochondrial inner membrane potential induces caspase 3 activation in cells⁴². Therefore, caspase 3 activation in tmpk mutant-expressing cells treated with AZT was next evaluated. Populations of F105Y- or R16GLL-expressing cells that were treated with AZT showed a significant increase (FIG. 17B) in the percentage of activated caspase 3-positive cells compared to populations of untreated cells (4.6±0.1 and 7.8±0.5-fold increases, respectively). No significant increases in the percentage of cells with activated caspase 3 were seen in the negative controls (NT and LV-IRES-huCD19Δ-transduced cells) following AZT incubation (FIG. 17B). Interestingly, tmpk WT-overexpressing cells that were treated with AZT showed a slight, but significant, increase of the percentage of active caspase 3-positive cells compared to untreated cells. Taken together, our data collectively demonstrates that the mechanism of the induction of apoptosis by AZT in the tmpk-mutant-expressing cells is the activation of caspase 3 resulting from the increase in the loss of the mitochondrial membrane potential, caused by the accumulation of AZT-TP in the cells.

### In Vivo Killing of LV Transduced Cells Mediated by AZT

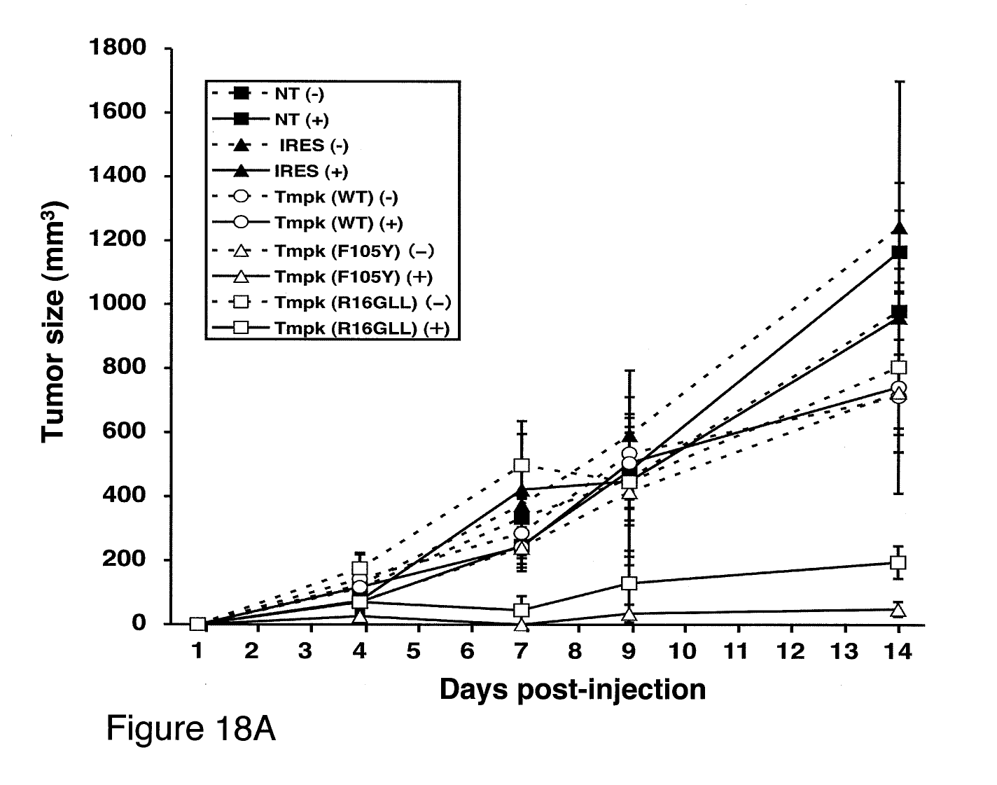

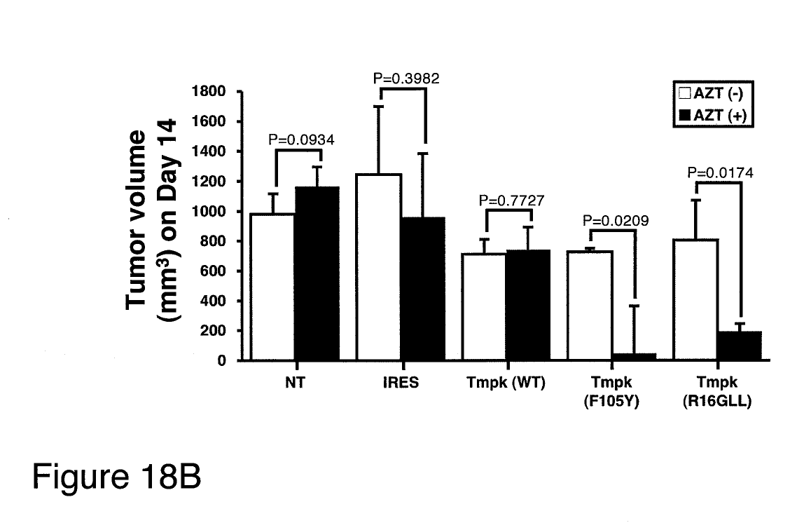

Next killing of the tmpk-mutant-expressing cells in an in vivo tumor model was examined. K562 erythroid leukemia cells were transduced with the LVs that engineered expression of IRES-huCD19Δ, WT tmpk, or a mutant form of the kinase (F105Y or R16GLL). Since the transduction efficiency of the F105Y LV into the K562 cells was fairly modest (68% of cells positive based on observed huCD19 expression; data not shown), these cells were enriched by FACS using anti-human CD19 conjugated to phycoerythrin (PE). After enrichment, the percentage of CD19-positive K562 cells was over 95% (data not shown). This also confirms the auxiliary utility of huCD19Δ as a cell surface marker enabling immuno-affinity enrichment of transduced cells. Growth characteristics of productively transduced K562 cells were then assessed. Minimal differences in growth of the LV-transduced cells were observed (data not shown). Next, 2×10⁷ transduced K562 cells were injected s.c. into the right flank of NOD/SCID mice. Starting one day after the cell injection, the mice received daily i.p. injections of AZT (2.5 mg/kg/day) or vehicle for two weeks. According to the UHN ACC SOP for humane endpoints, mice were sacrificed when the tumor burden reached ˜1.5 cm³. In animals injected with non-transduced K562 cells, this endpoint occurred within two weeks post-injection. Mice not receiving AZT treatment quickly developed large tumors in a time dependent manner (FIG. 18A). In contrast, the growth of K562 cells transduced with either of the tmpk mutant LVs (F105Y or R16GLL) was strongly inhibited (P=0.0209 and 0.0174, respectively) by daily AZT injection, and the effects were sustained over time (FIG. 18B). No significant tumor growth inhibition by AZT was observed in the LV-tmpk (WT)-IRES-huCD19Δ-transduced, LV-IRES-huCD19Δ-transduced, or the NT-cell injected mice (FIG. 7B18B).

### DISCUSSION

Here the inventors have shown that overexpression of rationally-designed mutant forms of human tmpk with improved kinetics significantly reduce cellular viability following AZT treatment both in vitro and in vivo and is useful for treating disease. In addition, these results show that the mechanism of AZT-induced apoptosis is associated with loss of mitochondrial inner membrane potential and activation of caspase 3 in the tmpk-mutant expressing cells. This mechanism provides significant advantages over previous suicide schemas and also allows for killing of non-dividing cells as shown in FIG. 4.

Tmpk is crucial for the activation of a series of prodrugs, including AZT, by catalyzing the second phosphorylation step. It has been shown that this is a rate-limiting step in the activation of AZT¹⁷, resulting in an accumulation of the intermediate metabolite, AZT-MP. AZT was the first effective treatment for AIDS patients¹³⁻¹⁵, however, long-term treatment with AZT has been reported to induce a severe myopathy characterized by structural and functional alterations in mitochondria as a result of accumulation of AZT-MP^{19,20,22}. Inhibition of the mitochondrial inner membrane potential has also been found in the muscle mitochondria of long-term AZT-treated rats²¹. The inventors have shown that accumulation of AZT-TP in the tmpk-mutant expressing cells abolished the inner membrane potential of mitochondria (FIG. 17A) and increased the apoptotic-index as a result of the activation of caspase 3 (FIG. 17B). Interestingly, these results revealed that while accumulation of AZT-MP in the tmpk (WT)-overexpressing cells did not affect the mitochondrial function (FIG. 17A), there was a slight induction of apoptosis in these cells mediated by AZT (FIG. 17B).

Another advantage of the invention is that it ensures that a high percentage of transduced cells, for example, cells to be transplanted, express the suicide gene. The use of huCD19Δ as a cell-surface marker increases the ratio of gene-modified cells by immuno-affinity enrichment. The contribution of the CD19 cytoplasmic domain in signal transduction has been assessed by others; in vitro by transfecting the cells with a truncated form of the human cDNA⁴³, and in vivo by using CD19-deficient mouse that expresses a transgene encoding the truncated human CD19³⁹. These studies demonstrated that the cytoplasmic domain of CD19 is a crucial for the signaling and for the in vivo function of the CD19/CD21/CD81/Leu-13 complex. This indicates that the truncated form of human CD19 that employed is unlikely to transmit a signal.

Adoptive immunotherapy using T cells is an efficient approach to treat hematological malignancies^{11,34,44-46}. GVHD, however, still remains a major problem following non-T cell-depleted allogeneic BMT⁴⁷. In addition to its utility in deleting gene-modified cells if they undergo transformative events, the inventors have shown that it would be advantageous to incorporate an efficient in vivo safety switch that would enable the elimination of gene-modified T cells in the event of GvHD. The drug GCV has been used to deplete HSV-tk-expressing allogeneic lymphocytes following BMT³⁴⁴⁴ Depletion is not always complete, however, and unwanted host immune responses against cells expressing this foreign enzyme can impair their function and persistence^{10,11}. In addition, T cell responses to multiple epitopes of HSV-tk suggests that modification of immunogenic sequences in HSV-tk would likely be ineffective in ablating this reaction¹¹. The use of human gene products as an alternative suicide gene in such situations is less likely to induce an immune response. Furthermore, most BMT patients are on prophylactic GCV to minimize CMV infections, which decreases the broad clinical utility of HSV-tk-based suicide gene therapy.

The inventors showed that the tmpk-mutant expressing Jurkat cells showed an increase in apoptotic index following AZT-treatment in vitro (FIGS. 14 and 15). NOD/SCID mice xenografted with LV-tmpk-mutant-transduced K562 cells (either F105Y or RG16LL) treated with AZT showed the suppression of tumor growth in vivo (FIG. 18). This data shows that the suicide gene methods of the invention eliminate unwanted cells in vivo, including cancer cells and allografted T cells.

### Example 16

### Lentigen pLTG690 TMPK Vector Construct—Use for Treating Disease

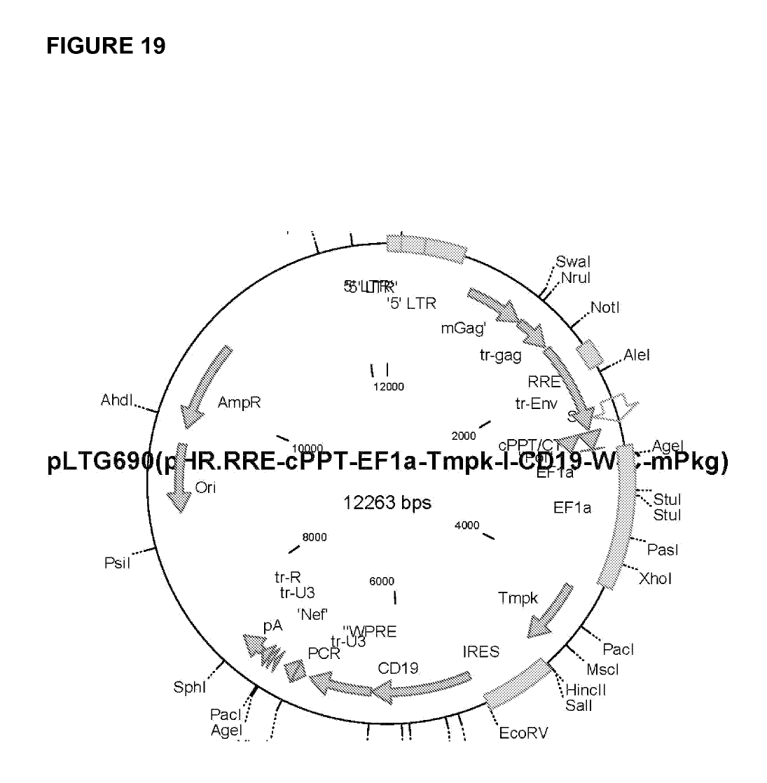

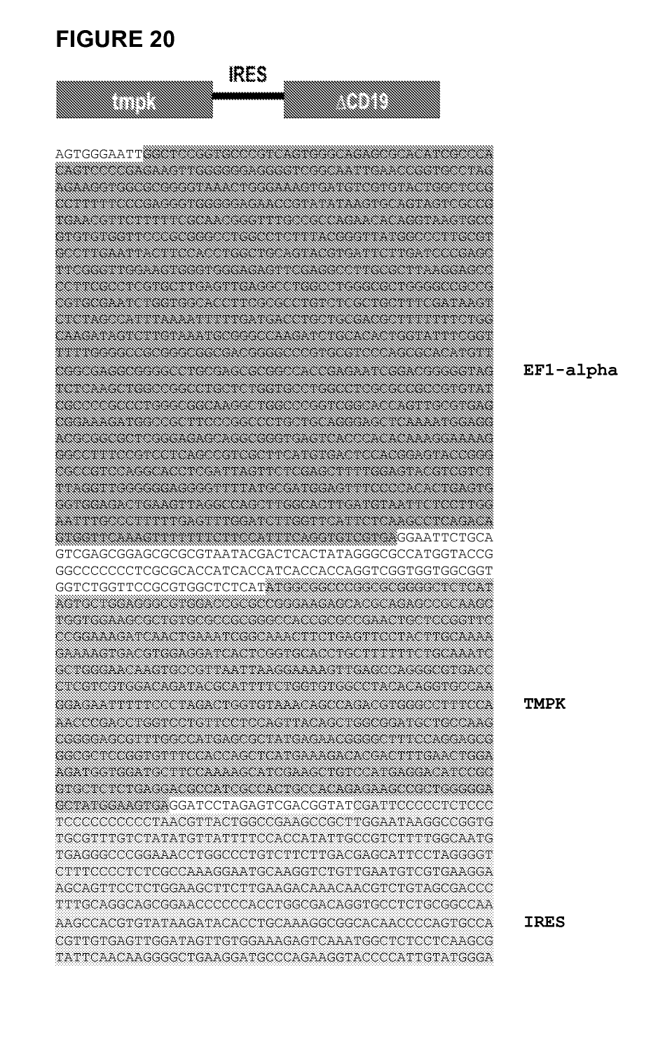

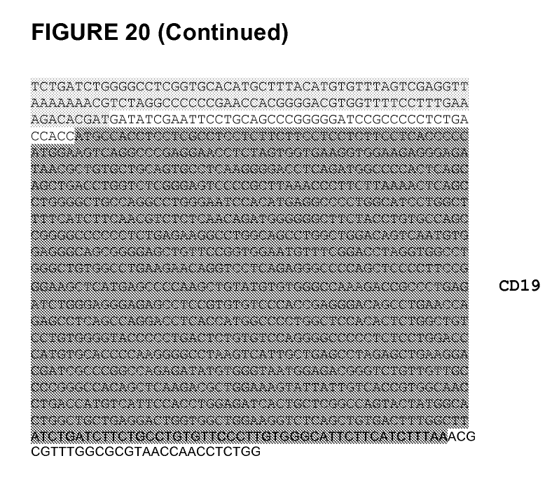

Tmpk IRES and truncated CD19 were cloned into a Lentigen vector pLTG690 construct providing pLTG690 (pHR.RRE-cPPT-EF1a-Tmpk-1-CD19-WC-mpkg) (also referred to as Lentigen TMPK-CD19 vector). The vector map and sequence of the tmpk IRES truncated CD19 genetic construct are shown in FIGS. 19 and 20 with features highlighted. Plasmid DNA was used to transfect and produce Lentiviral vector virus

Another candidate vector Lentigen-DNK/CD19 was also produced. Experiments were conducted using the Lentigen TMPK-CD19 vector construct, the previously described pHR′-cPPT-EF-IRES-huCD19Δ-W-SIN.

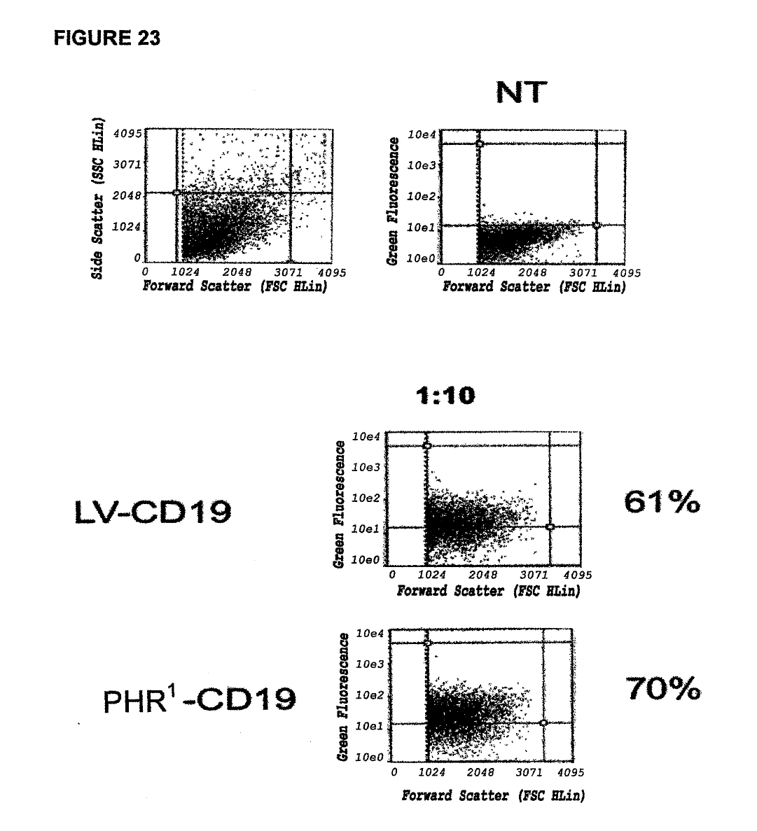

Vector constructs were used to transduce 293 cells, and the expression of the CD19 marker analyzed by FACS (FIG. 23; NT=non-transduced). Expression of the CD19 marker from the Lentigen TMPK-CD19 vector was comparable to that expressed from the pHR′ vector backbone (pHR′-cPPT-EF-IRES-huCD19Δ-W-SIN described above). Expression was determined by detection of CD19 by staining with PE conjugated mouse anti-human CD19. Virus was tittered using 293T cells by measuring CD19 expression using flow cytometry methods on transduced cells (and controls) two days after infections.

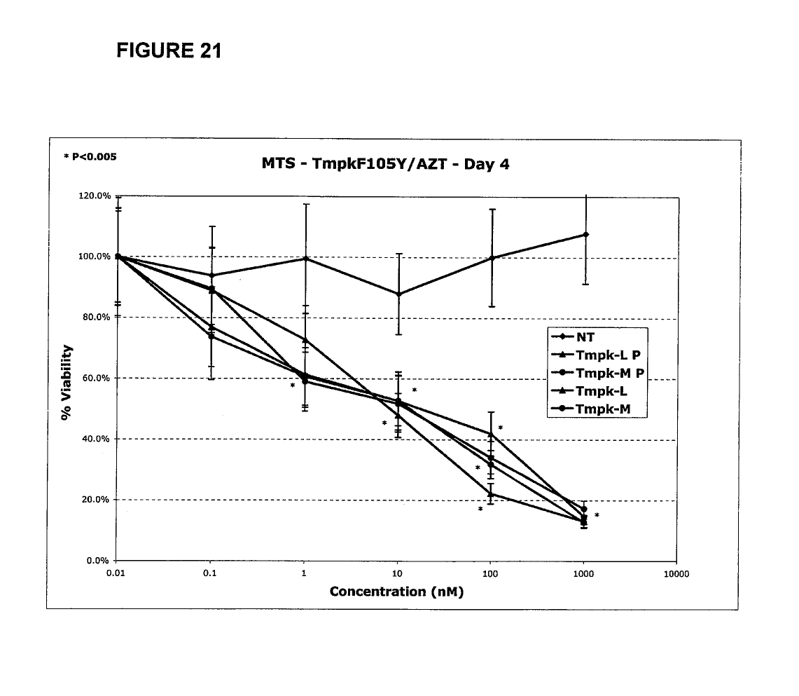

The function of tmpk was tested by assessing sensitivity to AZT (FIG. 21). Increasing concentrations of AZT induced dose dependent killing in transduced cells.

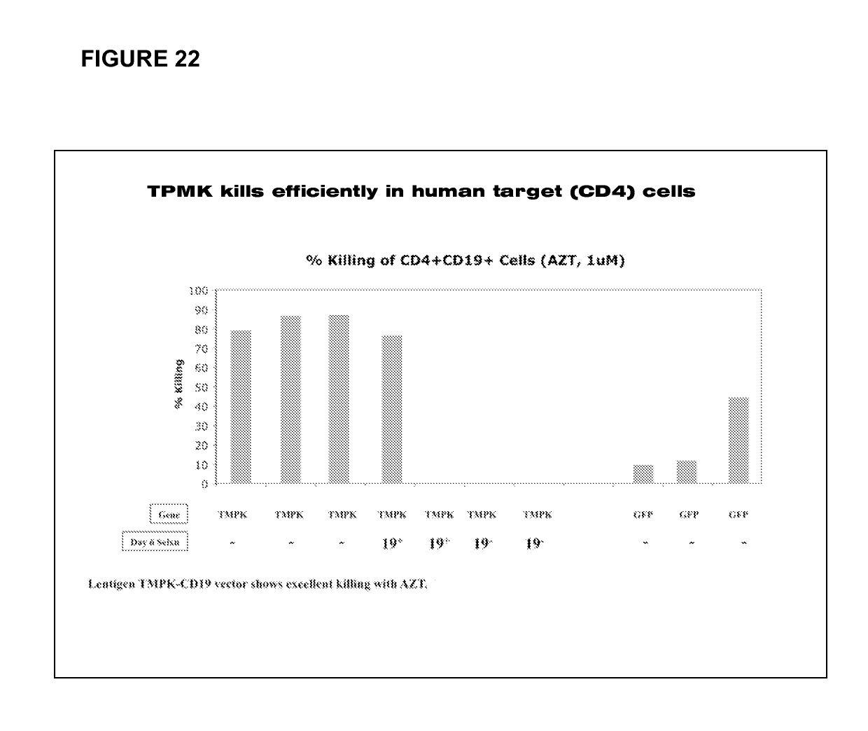

The Lentigen TMPK-CD19 vector was also used to transduce primary human T-cells. The cells were tested for CD19 expression, and sensitivity to AZT. The sensitivity was shown to be very high. FIG. 22 illustrates that greater than 80% of CD4+ CD19+ cells were killed by 1 micromolar AZT demonstrating that TMPK kills efficiently in human target (CD4) cells and provides key support for use in GVHD. Cell viability was assessed by MTS analysis and cell counting.

### Example 17

Vector produced by Lentigen (pHR′ backbone −tmpK/CD19, Lentigen-tmpK/CD19, Lentigen-DNK/CD19) will be titered on 293T cells. Titer will be measured by CD19 expression using flow cytometry methods on transduced cells (and controls) two days after infections. Next Jurkat cells will be infected. Expression of CD19 will be measured as above. Populations of productively-infected Jurkat cells will be enriched by flow cytometry methods for CD19 expression and also subjected to clonal isolation techniques. Enriched and clonal cell populations will be subjected to cell killing assays with various concentrations of added prodrugs. Here MTS analyses will be performed. Cells will also be cultured long-term to assess viability.

### Example 18

### Clinical Trial Protocol

A Lentiviral vector construct is readily made that includes at least one antigen and tmpk for the purpose of generating an immune response against the antigen. Lentiviral vectors are integrating vectors. tmpk significantly increases the safety of a vaccine contained in an integrating vector by providing the capability to kill the cells that contain the integrated vector that expresses the antigen. This increases the safety in a number of ways. First it removes the possibility that insertion of the integrating vector into the chromosome may result in a deleterious outcome for the patient as if such an event should occur. The cells containing the vector can be deleted with the use of the prodrug (eg AZT). Second, killing of the cells after the antigen has stimulated the immune response initially may further increase the potency of the immune response, as cells undergoing apoptosis can release antigen, further recruit immune mediators to the site of cell death and further stimulate the immune response. The invention is not limited to use only of an antigen and the tmpk gene, but it could also contain other co-stimulatory molecules such as CD40 ligand, interleukins (a non-limiting example being IL-12), Colony Stimulating Factors (a non limiting example being GM-CSF) and other proteins or factors that can increase the potency, duration, or durability of the immune response. The invention is not limited to use of integrating vectors, and non-integrating vector is also readily used, for example, to provide increased immunogenicity, safety or a desired effect can be enhanced with the use of the tmpk gene. A Lentiviral vector construct expressing the tmpk gene is readily used for the direct injection of tumors or for the transduction of cells that are predisposed to becoming tumor cells. One example is breast cancer. Certain patients have a high incidence of protracting breast cancer and the ductal epithelium of these patients (which are normal or pre-cancerous) are readily transduced with a Lentiviral vector (or any persisting vector). Since tmpk is not known to be immunogenic, the cells will behave normally. However, if the cells become cancerous in the future, then the cells are readily deleted (since they will be marked with the tmpk gene) with the administration of a prodrug.

Table of Sequences

```
<210> 1
<211> 639
<212> DNA
<213> Homo sapiens
<400> 1
atggcggccc ggcgcggggc tctcatagtg ctggagggcg tggaccgcgc cgggaagagc
60
acgcagagcc gcaagctggt ggaagcgctg tgcgccgcgg gccaccgcgc cgaactgctc
120
cggttcccgg aaagatcaac tgaaatcggc aaacttctga gttcctactt gcaaaagaaa
180
agtgacgtgg aggatcactc ggtgcacctg cttttttctg caaatcgctg ggaacaagtg
240
ccgttaatta aggaaaagtt gagccagggc gtgaccctcg tcgtggacag atacgcattt
300
tctggtgtgg ccttcaccgg tgccaaggag aatttttccc tagattggtg taaacagcca
360
gacgtgggcc ttcccaaacc cgacctggtc ctgttcctcc agttacagct ggcggatgct
420
gccaagcggg gagcgtttgg ccatgagcgc tatgagaacg gggctttcca ggagcgggcg
480
ctccggtgtt tccaccagct catgaaagac acgactttga actggaagat ggtggatgct
540
tccaaaagca tcgaagctgt ccatgaggac atccgcgtgc tctctgagga cgccatccgc
600
actgccacag agaagccgct gggggagcta tggaagtga
639
<210> 2
<211> 212
<212> PRT
<213> Homo sapiens
<400> 2
Met Ala Ala Arg Arg Gly Ala Leu Ile Val Leu Glu Gly Val Asp Arg
1 5 10 15
Ala Gly Lys Ser Thr Gln Ser Arg Lys Leu Val Glu Ala Leu Cys Ala
                        20 25 30
Ala Gly His Arg Ala Glu Leu Leu Arg Phe Pro Glu Arg Ser Thr Glu
                35 40 45
Ile Gly Lys Leu Leu Ser Ser Tyr Leu Gln Lys Lys Ser Asp Val Glu
        50 55 60
Asp His Ser Val His Leu Leu Phe Ser Ala Asn Arg Trp Glu Gln Val
65 70 75 80
Pro Leu Ile Lys Glu Lys Leu Ser Gln Gly Val Thr Leu Val Val Asp
                                85 90 95
Arg Tyr Ala Phe Ser Gly Val Ala Phe Thr Gly Ala Lys Glu Asn Phe
                        100 105 110
Ser Leu Asp Trp Cys Lys Gln Pro Asp Val Gly Leu Pro Lys Pro Asp
                115 120 125
Leu Val Leu Phe Leu Gln Leu Gln Leu Ala Asp Ala Ala Lys Arg Gly
        130 135 140
Ala Phe Gly His Glu Arg Tyr Glu Asn Gly Ala Phe Gln Glu Arg Ala
145 150 155 160
Leu Arg Cys Phe His Gln Leu Met Lys Asp Thr Thr Leu Asn Trp Lys
                                165 170 175
Met Val Asp Ala Ser Lys Ser Ile Glu Ala Val His Glu Asp Ile Arg
                        180 185 190
Val Leu Ser Glu Asp Ala Ile Arg Thr Ala Thr Glu Lys Pro Leu Gly
                195 200 205
Glu Leu Trp Lys
        210
<210> 3
<211> 639
<212> DNA
<213> Homo sapiens
<400> 3
atggcggccc ggcgcggggc tctcatagtg ctggagggcg tggaccgcgc cgggaagagc
60
acgcagagcc gcaagctggt ggaagcgctg tgcgccgcgg gccaccgcgc cgaactgctc
120
cggttcccgg aaagatcaac tgaaatcggc aaacttctga gttcctactt gcaaaagaaa
180
agtgacgtgg aggatcactc ggtgcacctg cttttttctg caaatcgctg ggaacaagtg
240
ccgttaatta aggaaaagtt gagccagggc gtgaccctcg tcgtggacag atacgcattt
300
tctggtgtgg ccttcaccgg tgccaaggag aatttttccc tagattggtg taaacagcca
360
gacgtgggcc ttcccaaacc cgacctggtc ctgttcctcc agttacagct ggcggatgct
420
gccaagcggg gagcgtttgg ccatgagcgc tatgagaacg gggctttcca ggagcgggcg
480
ctccggtgtt tccaccagct catgaaagac acgactttga actggaagat ggtggatgct
540
tccaaaagca tcgaagctgt ccatgaggac atccgcgtgc tctctgagga cgccatccgc
600
actgccacag agaagccgct gggggagcta tggaagtga
639
<210> 4
<211> 212
<212> PRT
<213> Homo sapiens
<400> 4
Met Ala Ala Arg Arg Gly Ala Leu Ile Val Leu Glu Gly Val Asp Arg
1 5 10 15
Ala Gly Lys Ser Thr Gln Ser Arg Lys Leu Val Glu Ala Leu Cys Ala
                        20 25 30
Ala Gly His Arg Ala Glu Leu Leu Arg Phe Pro Glu Arg Ser Thr Glu
                35 40 45
Ile Gly Lys Leu Leu Ser Ser Tyr Leu Gln Lys Lys Ser Asp Val Glu
        50 55 60
Asp His Ser Val His Leu Leu Phe Ser Ala Asn Arg Trp Glu Gln Val
65 70 75 80
Pro Leu Ile Lys Glu Lys Leu Ser Gln Gly Val Thr Leu Val Val Asp
                                85 90 95
Arg Tyr Ala Phe Ser Gly Val Ala Phe Thr Gly Ala Lys Glu Asn Phe
                        100 105 110
Ser Leu Asp Trp Cys Lys Gln Pro Asp Val Gly Leu Pro Lys Pro Asp
                115 120 125
Leu Val Leu Phe Leu Gln Leu Gln Leu Ala Asp Ala Ala Lys Arg Gly
        130 135 140
Ala Phe Gly His Glu Arg Tyr Glu Asn Gly Ala Phe Gln Glu Arg Ala
145 150 155 160
Leu Arg Cys Phe His Gln Leu Met Lys Asp Thr Thr Leu Asn Trp Lys
                                165 170 175
Met Val Asp Ala Ser Lys Ser Ile Glu Ala Val His Glu Asp Ile Arg
                        180 185 190
Val Leu Ser Glu Asp Ala Ile Arg Thr Ala Thr Glu Lys Pro Leu Gly
                195 200 205
Glu Leu Trp Lys
        210
<210> 5
<211> 636
<212> DNA
<213> Homo sapiens
<400> 5
atggcggccc ggcgcggggc tctcatagtg ctggagggcg tggaccgcgc cgggaagagc
60
acgcagagcc gcaagctggt ggaagcgctg tcgcgcgggc caccgcccga actgctccgg
120
ttcccggaaa gatcaactga aatcggcaaa cttctgagtt cctacttgca aaagaaaagt
180
gacgtggagg atcactcggt gcacctgctt ttttctgcaa atcgctggga acaagtgccg
240
ttaattaagg aaaagttgag ccagggcytg accctcgtcg tggacagata cgcattttct
300
ggtgtggcct tcaccggtgc caaggagaat ttttccctag actggtgtaa acagccagac
360
gtgggccttc ccaaacccga cctggtcctg ttcctccagt tacagctggc ggatgctgcc
420
aagcggggag cgtttggcca tgagcgctat gagaacgggg ctttccagga gcgggcgctc
480
cggtgtttcc accagctcat gaaagacacg actttgaact ggaagatggt ggatgcttcc
540
aaaagactcg aagctgtcca tgaggaactc cgcgtgctct ctgaggacgc catccgcact
600
gccacagaga agccgctggg ggagctatgg aagtga
636
<210> 6
<211> 211
<212> PRT
<213> Homo sapiens
<400> 6
Met Ala Ala Arg Arg Gly Ala Leu Ile Val Leu Glu Gly Val Asp Arg
1 5 10 15
Ala Gly Lys Ser Thr Gln Ser Arg Lys Leu Val Glu Ala Leu Ser Arg
                        20 25 30
Gly Pro Pro Pro Glu Leu Leu Arg Phe Pro Glu Arg Ser Thr Glu Ile
                35 40 45
Gly Lys Leu Leu Ser Ser Tyr Leu Gln Lys Lys Ser Asp Val Glu Asp
        50 55 60
His Ser Val His Leu Leu Phe Ser Ala Asn Arg Trp Glu Gln Val Pro
65 70 75 80
Leu Ile Lys Glu Lys Leu Ser Gln Gly Val Thr Leu Val Val Asp Arg
                                85 90 95
Tyr Ala Phe Ser Gly Val Ala Phe Thr Gly Ala Lys Glu Asn Phe Ser
                        100 105 110
Leu Asp Trp Cys Lys Gln Pro Asp Val Gly Leu Pro Lys Pro Asp Leu
                115 120 125
Val Leu Phe Leu Gln Leu Gln Leu Ala Asp Ala Ala Lys Arg Gly Ala
        130 135 140
Phe Gly His Glu Arg Tyr Glu Asn Gly Ala Phe Gln Glu Arg Ala Leu
145 150 155 160
Arg Cys Phe His Gln Leu Met Lys Asp Thr Thr Leu Asn Trp Lys Met
                                165 170 175
Val Asp Ala Ser Lys Arg Leu Glu Ala Val His Glu Glu Leu Arg Val
                        180 185 190
Leu Ser Glu Asp Ala Ile Arg Thr Ala Thr Glu Lys Pro Leu Gly Glu
                195 200 205
Leu Trp Lys
        210
<210> 7
<211> 639
<212> DNA
<213> Homo sapiens
<400> 7
atggcggccc ggcgcggggc tctcatagtg ctggagggcg tggaccgcgc cgggaagagc
60
acgcagagcc gcaagctggt ggaagcgctg tgcgccgcgg gccaccgcgc cgaactgctc
120
cggttcccgg aaagatcaac tgaaatcggc aaacttctga gttcctactt gcaaaagaaa
180
agtgacgtgg aggatcactc ggtgcacctg cttttttctg caaatcgctg ggaacaagtg
240
ccgttaatta aggaaaagtt gagccagggc gtgaccctcg tcgtggacag atacgcattt
300
tctggtgtgg ccttcaccgg tgccaaggag aatttttccc tagattggtg taaacagcca
360
gacgtgggcc ttcccaaacc cgacctggtc ctgttcctcc agttacagct ggcggatgct
420
gccaagcggg gagcgtttgg ccatgagcgc tatgagaacg gggctttcca ggagcgggcg
480
ctccggtgtt tccaccagct catgaaagac acgactttga actggaagat ggtggatgct
540
tccaaaagca tcgaagctgt ccatgaggac atccgcgtgc tctctgagga cgccatccgc
600
actgccacag agaagccgct gggggagcta tggaaggac
639
<210> 8
<211> 213
<212> PRT
<213> Homo sapiens
<400> 8
Met Ala Ala Arg Arg Gly Ala Leu Ile Val Leu Glu Gly Val Asp Arg
1 5 10 15
Ala Gly Lys Ser Thr Gln Ser Arg Lys Leu Val Glu Ala Leu Cys Ala
                        20 25 30
Ala Gly His Arg Ala Glu Leu Leu Arg Phe Pro Glu Arg Ser Thr Glu
                35 40 45
Ile Gly Lys Leu Leu Ser Ser Tyr Leu Gln Lys Lys Ser Asp Val Glu
        50 55 60
Asp His Ser Val His Leu Leu Phe Ser Ala Asn Arg Trp Glu Gln Val
65 70 75 80
Pro Leu Ile Lys Glu Lys Leu Ser Gln Gly Val Thr Leu Val Val Asp
                                85 90 95
Arg Tyr Ala Phe Ser Gly Val Ala Phe Thr Gly Ala Lys Glu Asn Phe
                        100 105 110
Ser Leu Asp Trp Cys Lys Gln Pro Asp Val Gly Leu Pro Lys Pro Asp
                115 120 125
Leu Val Leu Phe Leu Gln Leu Gln Leu Ala Asp Ala Ala Lys Arg Gly
        130 135 140
Ala Phe Gly His Glu Arg Tyr Glu Asn Gly Ala Phe Gln Glu Arg Ala
145 150 155 160
Leu Arg Cys Phe His Gln Leu Met Lys Asp Thr Thr Leu Asn Trp Lys
                                165 170 175
Met Val Asp Ala Ser Lys Ser Ile Glu Ala Val His Glu Asp Ile Arg
                        180 185 190
Val Leu Ser Glu Asp Ala Ile Arg Thr Ala Thr Glu Lys Pro Leu Gly
                195 200 205
Glu Leu Trp Lys Asp
        210
<210> 9
<211> 639
<212> DNA
<213> Mus musculus
<400> 9
atggcgtcgc gtcggggagc gctcatcgtg ctggagggtg tggaccgtgc tggcaagacc
60
acgcagggcc tcaagctggt gaccgcgctg tgcgcctcgg gccacagagc ggagctgctg
120
cgtttccccg aaagatcaac ggaaatcggc aagcttctga attcctactt ggaaaagaaa
180
acggaactag aggatcactc cgtgcacctg ctcttctctg caaaccgctg ggaacaagta
240
ccattaatta aggcgaagtt gaaccagggt gtgacccttg ttttggacag atacgccttt
300
tctggggttg ccttcactgg tgccaaagag aatttttccc tggattggtg taaacaaccg
360
gacgtgggcc ttcccaaacc tgacctgatc ctgttccttc agttacaatt gctggacgct
420
gctgcacggg gagagtttgg ccttgagcga tatgagaccg ggactttcca aaagcaggtt
480
ctgttgtgtt tccagcagct catggaagag aaaaacctca actggaaggt ggttgatgct
540
tccaaaagca ttgaggaagt ccataaagaa atccgtgcac actctgagga cgccatccga
600
aacgctgcac agaggccact gggggagcta tggaaataa
639
<210> 10
<211> 212
<212> PRT
<213> Mus musculus
<400> 10
Met Ala Ser Arg Arg Gly Ala Leu Ile Val Leu Glu Gly Val Asp Arg
1 5 10 15
Ala Gly Lys Thr Thr Gln Gly Leu Lys Leu Val Thr Ala Leu Cys Ala
                        20 25 30
Ser Gly His Arg Ala Glu Leu Leu Arg Phe Pro Glu Arg Ser Thr Glu
                35 40 45
Ile Gly Lys Leu Leu Asn Ser Tyr Leu Glu Lys Lys Thr Glu Leu Glu
        50 55 60
Asp His Ser Val His Leu Leu Phe Ser Ala Asn Arg Trp Glu Gln Val
65 70 75 80
Pro Leu Ile Lys Ala Lys Leu Asn Gln Gly Val Thr Leu Val Leu Asp
                                85 90 95
Arg Tyr Ala Phe Ser Gly Val Ala Phe Thr Gly Ala Lys Glu Asn Phe
                        100 105 110
Ser Leu Asp Trp Cys Lys Gln Pro Asp Val Gly Leu Pro Lys Pro Asp
                115 120 125
Leu Ile Leu Phe Leu Gln Leu Gln Leu Leu Asp Ala Ala Ala Arg Gly
        130 135 140
Glu Phe Gly Leu Glu Arg Tyr Glu Thr Gly Thr Phe Gln Lys Gln Val
145 150 155 160
Leu Leu Cys Phe Gln Gln Leu Met Glu Glu Lys Asn Leu Asn Trp Lys
                                165 170 175
Val Val Asp Ala Ser Lys Ser Ile Glu Glu Val His Lys Glu Ile Arg
                        180 185 190
Ala His Ser Glu Asp Ala Ile Arg Asn Ala Ala Gln Arg Pro Leu Gly
                195 200 205
Glu Leu Trp Lys
        210
<210> 11
<211> 212
<212> PRT
<213> Homo sapiens
<400> 11
Met Ala Ala Arg Arg Gly Ala Leu Ile Val Leu Glu Gly Val Asp Arg
1 5 10 15
Ala Gly Lys Ser Thr Gln Ser Arg Lys Leu Val Glu Ala Leu Cys Ala
                        20 25 30
Ala Gly His Arg Ala Glu Leu Leu Arg Pho Pro Glu Arg Ser Thr Glu
                35 40 45
Ile Gly Lys Leu Leu Ser Ser Tyr Leu Gln Lys Lys Ser Asp Val Glu
        50 55 60
Asp His Ser Val His Leu Leu Phe Ser Ala Asn Arg Trp Glu Gln Val
65 70 75 80
Pro Leu Ile Lys Glu Lys Leu Ser Gln Gly Val Thr Leu Val Val Asp
                                85 90 95
Arg Tyr Ala Phe Ser Gly Val Ala Tyr Thr Gly Ala Lys Glu Asn Phe
                        100 105 110
Ser Leu Asp Trp Cys Lys Gln Pro Asp Val Gly Leu Pro Lys Pro Asp
                115 120 125
Leu Val Leu Pho Leu Gln Leu Gln Leu Ala Asp Ala Ala Lys Arg Gly
        130 135 140
Ala Phe Gly His Glu Arg Tyr Glu Asn Gly Ala Phe Gln Glu Arg Ala
145 150 155 160
Leu Arg Cys Phe His Gln Leu Met Lys Asp Thr Thr Leu Asn Trp Lys
                                165 170 175
Met Val Asp Ala Ser Lys Ser Ile Glu Ala Val His Glu Asp Ile Arg
                        180 185 190
Val Leu Ser Glu Asp Ala Ile Arg Thr Ala Thr Glu Lys Pro Leu Gly
                195 200 205
Glu Leu Trp Lys
        210
<210> 12
<211> 214
<212> PRT
<213> Homo sapiens
<400> 12
Met Ala Ala Arg Arg Gly Ala Leu Ile Val Leu Glu Gly Val Asp Gly
1 5 10 15
Ala Gly Lys Ser Thr Gln Ser Arg Lys Leu Val Glu Ala Leu Cys Ala
                        20 25 30
Ala Gly His Arg Ala Glu Leu Leu Arg Phe Pro Glu Arg Ser Thr Glu
                35 40 45
Ile Gly Lys Leu Leu Ser Ser Tyr Leu Gln Lys Lys Ser Asp Val Glu
        50 55 60
Asp His Ser Val His Leu Leu Phe Ser Ala Asn Arg Trp Glu Gln Val
65 70 75 80
Pro Leu Ile Lys Glu Lys Leu Ser Gln Gly Val Thr Leu Val Val Asp
                                85 90 95
Arg Tyr Ala Phe Ser Gly Val Ala Phe Thr Gly Ala Lys Glu Asn Phe
                        100 105 110
Ser Leu Asp Trp Cys Lys Gln Pro Asp Val Gly Leu Pro Lys Pro Asp
                115 120 125
Leu Val Leu Phe Leu Gln Leu Thr Pro Glu Val Gly Leu Lys Arg Ala
        130 135 140
Arg Ala Arg Gly Gln Leu Asp Arg Tyr Glu Asn Gly Ala Phe Gln Glu
145 150 155 160
Arg Ala Leu Arg Cys Phe His Gln Leu Met Lys Asp Thr Thr Leu Asn
                                165 170 175
Trp Lys Met Val Asp Ala Ser Lys Ser Ile Glu Ala Val His Glu Asp
                        180 185 190
Ile Arg Val Leu Ser Glu Asp Ala Ile Ala Thr Ala Thr Glu Lys Pro
                195 200 205
Leu Gly Glu Leu Trp Lys
        210
<210> 13
<211> 6811
<212> DNA
<213> Vector
<400> 13
tggaagggct aattcactcc caacgaagac aagatatcct tgatctgtgg atctaccaca
60
cacaaggcta cttccctgat tggcagaact acacaccagg accagggatc agatatccac
120
tgacctttgg atggtgctac aagctagtac cagttgagcc agataaggta gaagaggcca
180
acaaaggaga gaacaccagc ttgttacacc ctgtgagcct gcatggaatg gatgacccgg
240
agagagaagt gttagagtgg aggtttgaca gccgcctagc atttcatcac gtggcccgag
300
agctgcatcc ggagtacttc aagaactgct gatatcgagc ttgctacaag ggactttccg
360
ctggggactt tccagggagg cgtggcctgg gcgggactgg ggagtggcga gccctcagat
420
gctgcatata agcagctgct ttttgcctgt actgggtctc tctggttaga ccagatctga
480
gcctgggagc tctctggcta actagggaac ccactgctta agcctcaata aagcttgcct
540
tgagtgcttc aagtagtgtg tgcccgtctg ttgtgtgact ctggtaacta gagatccctc
600
agaccctttt agtcagtgtg gaaaatctct agcagtggcg cccgaacagg gacttgaaag
660
cgaaagggaa accagaggag ctctctcgac gcaggactcg gcttgctgaa gcgcgcacgg
720
caagaggcga ggggcggcga ctggtgagta cgccaaaaat tttgactagc ggaggctaga
780
aggagagaga tgggtgcgag agcgtcagta ttaagcgggg gagaattaga tcgcgatggg
840
aaaaaattcg gttaaggcca gggggaaaga aaaaatataa attaaaacat atagtatggg
900
caagcaggga gctagaacga ttcgcagtta atcctggcct gttagaaaca tcagaaggct
960
gtagacaaat actgggacag ctacaaccat cccttcagac aggatcagaa gaacttagat
1020
cattatataa tacagtagca accctctatt gtgtgcatca aaggatagag ataaaagaca
1080
ccaaggaagc tttagacaag atagaggaag agcaaaacaa aagtaagacc accgcacagc
1140
aagcggccgc tgatcttcag acctggagga ggagatatga gggacaattg gagaagtgaa
1200
ttatataaat ataaagtagt aaaaattgaa ccattaggag tagcacccac caaggcaaag
1260
agaagagtgg tgcagagaga aaaaagagca gtgggaatag gagctttgtt ccttgggttc
1320
ttgggagcag caggaagcac tatgggcgca gcgtcaatga cgctgacggt acaggccaga
1380
caattattgt ctggtatagt gcagcagcag aacaatttgc tgagggctat tgaggcgcaa
1440
cagcatctgt tgcaactcac agtctggggc atcaagcagc tccaggcaag aatcctggct
1500
gtggaaagat acctaaagga tcaacagctc ctggggattt ggggttgctc tggaaaactc
1560
atttgcacca ctgctgtgcc ttggaatgct agttggagta ataaatctct ggaacagatt
1620
tggaatcaca cgacctggat ggagtgggac agagaaatta acaattacac aagcttaata
1680
cactccttaa ttgaagaatc gcaaaaccag caagaaaaga atgaacaaga attattggaa
1740
ttagataaat gggcaagttt gtggaattgg tttaacataa caaattggct gtggtatata
1800
aaattattca taatgatagt aggaggcttg gtaggtttaa gaatagtttt tgctgtactt
1860
tctatagtga atagagttag gcagggatat tcaccattat cgtttcagac ccacctccca
1920
accccgaggg gacccgacag gcccgaagga atagaagaag aaggtggaga gagagacaga
1980
gacagatcca ttcgattagt gaacggatct cgacggtatc gcttttaaaa gaaaaggggg
2040
gattgggggg tacagtgcag gggaaagaat agtagacata atagcaacag acatacaaac
2100
taaagaatta caaaaacaaa ttacaaaaat tcaaaatttt atcgataagc tttgcaaaga
2160
tggataaagt tttaaacaga gaggaatctt tgcagctaat ggaccttcta ggtcttgaaa
2220
ggagtgggaa ttggctccgg tgcccgtcag tgggcagagc gcacatcgcc cacagtcccc
2280
gagaagttgg ggggaggggt cggcaattga accggtgcct agagaaggtg gcgcggggta
2340
aactgggaaa gtgatgtcgt gtactggctc cgcctttttc ccgagggtgg gggagaaccg
2400
tatataagtg cagtagtcgc cgtgaacgtt ctttttcgca acgggtttgc cgccagaaca
2460
caggtaagtg ccgtgtgtgg ttcccgcggg cctggcctct ttacgggtta tggcccttgc
2520
gtgccttgaa ttacttccac gcccctggct gcagtacgtg attcttgatc ccgagcttcg
2580
ggttggaagt gggtgggaga gttcgaggcc ttgcgcttaa ggagcccctt cgcctcgtgc
2640
ttgagttgag gcctggcctg ggcgctgggg ccgccgcgtg cgaatctggt ggcaccttcg
2700
cgcctgtctc gctgctttcg ataagtctct agccatttaa aatttttgat gacctgctgc
2760
gacgcttttt ttctggcaag atagtcttgt aaatgcgggc caagatctgc acactggtat
2820
ttcggttttt ggggccgcgg gcggcgacgg ggcccgtgcg tcccagcgca catgttcggc
2880
gaggcggggc ctgcgagcgc ggccaccgag aatcggacgg gggtagtctc aagctggccg
2940
gcctgctctg gtgcctggcc tcgcgccgcc gtgtatcgcc ccgccctggg cggcaaggct
3000
ggcccggtcg gcaccagttg cgtgagcgga aagatggccg cttcccggcc ctgctgcagg
3060
gagctcaaaa tggaggacgc ggcgctcggg agagcgggcg ggtgagtcac ccacacaaag
3120
gaaaagggcc tttccgtcct cagccgtcgc ttcatgtgac tccacggagt accgggcgcc
3180
gtccaggcac ctcgattagt tctcgagctt ttggagtacg tcgtctttag gttgggggga
3240
ggggttttat gcgatggagt ttccccacac tgagtgggtg gagactgaag ttaggccagc
3300
ttggcacttg atgtaattct ccttggaatt tgcccttttt gagtttggat cttggttcat
3360
tctcaagcct cagacagtgg ttcaaagttt ttttcttcca tttcaggtgt cgtgagagga
3420
attctgcagt cgagcggagc gcgcgtaata cgactcacta tagggcgcca tgggtaccgg
3480
gccccccctc gatcgaacaa caacaacaat aacacatggt tccgcgtggc tctcatatgg
3540
cggcccggcg cggggctctc atagtgctgg agggcgtgga cggcgccggg aagagcacgc
3600
agagccgcaa gctggtggaa gcgctgtgcg ccgcgggcca ccgcgccgaa ctgctccggt
3660
tcccggaaag atcaactgaa atcggcaaac ttctgagttc ctacttgcaa aagaaaagtg
3720
acgtggagga tcactcggtg cacctgcttt tttctgcaaa tcgctgggaa caagtgccgt
3780
taattaagga aaagttgagc cagggcgtga ccctcgtcgt ggacagatac gcattttctg
3840
gtgtggcctt caccggtgcc aaggagaatt tttccctaga ctggtgtaaa cagccagacg
3900
tgggccttcc caaacccgac ctggtcctgt tcctgcagtt aactccggaa gttggcttaa
3960
aacgcgcacg tgctcgcggc gagcttgacc gctatgagaa cggggctttc caggagcggg
4020
cgctccggtg tttccaccag ctcatgaaag acacgacttt gaactggaag atggtggatg
4080
cttccaaaag catcgaagct gtccatgagg acatccgcgt gctctctgag gacgccatcg
4140
ccactgccac agagaagccg ctgggggagc tatggaagtg aggatcagtc gacggtatcg
4200
attccccctc tccctccccc ccccctaacg ttactggccg aagccgcttg gaataaggcc
4260
ggtgtgcgtt tgtctatatg ttattttcca ccatattgcc gtcttttggc aatgtaggg
4320
cccggaaacc tggccctgtc ttcttgacga gcattcctag gggtctttcc cctctcgcca
4380
aaggaatgca aggtctgttg aatgtcgtga aggaagcagt tcctctggaa gcttcttgaa
4440
gacaaacaac gtctgtagcg accctttgca ggcagcggaa ccccccacct ggcgacaggt
4500
gcctctgcgg ccaaaagcca cgtgtataag atacacctgc aaaggcggca caaccccagt
4560
gccacgttgt gagttggata gttgtggaaa gagtcaaatg gctctcctca agcgtattca
4620
acaaggggct gaaggatgcc cagaaggtac cccattgtat gggatctgat ctggggcctc
4680
ggtgcacatg ctttacgtgt gtttagtcga ggttaaaaaa cgtctaggcc ccccgaacca
4740
cggggactg gttttccttt gaaaaacacg atgatatcga attcctgcag cccgggggat
4800
ccgccccctc tgaccaccat gccacctcct cgcctcctct tcttcctcct cttcctcacc
4860
cccatggaag tcaggcccga ggaacctcta gtggtgaagg tggaagaggg agataacgct
4920
gtgctgcagt gcctcaaggg gacctcagat ggccccactc agcagctgac ctggtctcgg
4980
gagtccccgc ttaaaccctt cttaaaactc agcctggggc tgccaggcct gggaatccac
5040
atgaggcccc tggcatcctg gcttttcatc ttcaacgtct ctcaacagat ggggggcttc
5100
tacctgtgcc agccggggcc CCCctctgag aaggcctggc agcctggctg gacagtcaat
5160
gtggagggca gcggggagct gttccggtgg aatgtttcgg acctaggtgg cctgggctgt
5220
ggcctgaaga acaggtcctc agagggcccc agctcccctt ccgggaagct catgagcccc
5280
aagctgtatg tgtgggccaa agaccgccct gagatctggg agggagagcc tccgtgtgtc
5340
ccaccgaggg acagcctgaa ccagagcctc agccaggacc tcaccatggc ccctggctcc
5400
acactctggc tgtcctgtgg ggtaccccct gactctgtgt ccaggggccc cctctcctgg
5460
acccatgtgc accccaaggg gcctaagtca ttgctgagcc tagagctgaa ggacgatcgc
5520
ccggccagag atatgtgggt aatggagacg ggtctgttgt tgccccgggc cacagctcaa
5580
gacgctggaa agtattattg tcaccgtggc aacctgacca tgtcattcca cctggagatc
5640
actgctcggc cagtactatg gcactggctg ctgaggactg gtggctggaa ggtctcagct
5700
gtgactttgg cttatctgat cttctgcctg tgttcccttg tgggcattct tcatctttaa
5760
ggcgcgcccc gggatccaag cttcaattgt ggtcactcga caatcaacct ctggattaca
5820
aaatttgtga aagattgact ggtattctta actatgttgc tccttttacg ctatgtggat
5880
acgctgcttt aatgcctttg tatcatgcta ttgcttcccg tatggctttc attttctcct
5940
ccttgtataa atcctggttg ctgtctcttt atgaggagtt gtggcccgtt gtcaggcaac
6000
gtggcgtggt gtgcactgtg tttgctgacg caacccccac tggttggggc attgccacca
6060
cctgtcagct cctttccggg actttcgctt tccccctccc tattgccacg gcggaactca
6120
tcgccgcctg ccttgcccgc tgctggacag gggctcggct gttgggcact gacaattccg
6180
tggtgttgtc ggggaagctg acgtcctttc catggctgct cgcctgtgtt gccacctgga
6240
ttctgcgcgg gacgtccttc tgctacgtcc cttcggccct caatccagcg gaccttcctt
6300
cccgcggcct gctgccggct ctgcggcctc ttccgcgtct tcgccttcgc cctcagacga
6360
gtcggatctc cctttgggcc gcctccccgc ctgtctcgag acctagaaaa acatggagca
6420
atcacaagta gcaatacagc agctaccaat gctgattgtg cctggctaga agcacaagag
6480
gaggaggagg tgggttttcc agtcacacct caggtacctt taagaccaat gacttacaag
6540
gcagatctta gccacttttt aaaagaaaag gggggactgg aagggctaat tcactcccaa
6600
cgaagacaag atctgctttt tgcttgtact gggtctctct ggttagacca gatctgagcc
6660
tgggagctct ctggctaact agggaaccca ctgcttaagc ctcaataaag cttgccttga
6720
gtgcttcaag tagtgtgtgc ccgtctgttg tgtgactctg gtaactagag atccctcaga
6780
cccttttagt cagtgtggaa aatctctagc a
6811
<210> 14
<211> 6805
<212> DNA
<213> Vector
<400> 14
tggaagggct aattcactcc caacgaagac aagatatcct tgatctgtgg atctaccaca
60
cacaaggcta cttccctgat tggcagaact acacaccagg accagggatc agatatccac
120
tgacctttgg atggtgctac aagctagtac cagttgagcc agataaggta gaagaggcca
180
acaaaggaga gaacaccagc ttgttacacc ctgtgagcct gcatggaatg gatgacccgg
240
agagagaagt gttagagtgg aggtttgaca gccgcctagc atttcatcac gtggcccgag
300
agctgcatcc ggagtacttc aagaactgct gatatcgagc ttgctacaag ggactttccg
360
ctggggactt tccagggagg cgtggcctgg gcgggactgg ggagtggcga gccctcagat
420
gctgcatata agcagctgct ttttgcctgt actgggtctc tctggttaga ccagatctga
480
gcctgggagc tctctggcta actagggaac ccactgctta agcctcaata aagcttgcct
540
tgagtgcttc aagtagtgtg tgcccgtctg ttgtgtgact ctggtaacta gagatccctc
600
agaccctttt agtcagtgtg gaaaatctct agcagtggcg cccgaacagg gacttgaaag
660
cgaaagggaa accagaggag ctctctcgac gcaggactcg gcttgctgaa gcgcgcacgg
720
caagaggcga ggggcggcga ctggtgagta cgccaaaaat tttgactagc ggaggctaga
780
aggagagaga tgggtgcgag agcgtcagta ttaagcgggg gagaattaga tcgcgatggg
840
aaaaaattcg gttaaggcca gggggaaaga aaaaatataa attaaaacat atagtatggg
900
caagcaggga gctagaacga ttcgcagtta atcctggcct gttagaaaca tcagaaggct
960
gtagacaaat actgggacag ctacaaccat cccttcagac aggatcagaa gaacttagat
1020
cattatataa tacagtagca accctctatt gtgtgcatca aaggatagag ataaaagaca
1080
ccaaggaagc tttagacaag atagaggaag agcaaaacaa aagtaagacc accgcacagc
1140
aagcggccgc tgatcttcag acctggagga ggagatatga gggacaattg gagaagtgaa
1200
ttatataaat ataaagtagt aaaaattgaa ccattaggag tagcacccac caaggcaaag
1260
agaagagtgg tgcagagaga aaaaagagca gtgggaatag gagctttgtt ccttgggttc
1320
ttgggagcag caggaagcac tatgggcgca gcgtcaatga cgctgacggt acaggccaga
1380
caattattgt ctggtatagt gcagcagcag aacaatttgc tgagggctat tgaggcgcaa
1440
cagcatctgt tgcaactcac agtctggggc atcaagcagc tccaggcaag aatcctggct
1500
gtggaaagat acctaaagga tcaacagctc ctggggattt ggggttgctc tggaaaactc
1560
atttgcacca ctgctgtgcc ttggaatgct agttggagta ataaatctct ggaacagatt
1620
tggaatcaca cgacctggat ggagtgggac agagaaatta acaattacac aagcttaata
1680
cactccttaa ttgaagaatc gcaaaaccag caagaaaaga atgaacaaga attattggaa
1740
ttagataaat gggcaagttt gtggaattgg tttaacataa caaattggct gtggtatata
1800
aaattattca taatgatagt aggaggcttg gtaggtttaa gaatagtttt tgctgtactt
1860
tctatagtga atagagttag gcagggatat tcaccattat cgtttcagac ccacctccca
1920
accccgaggg gacccgacag gcccgaagga atagaagaag aaggtggaga gagagacaga
1980
gacagatcca ttcgattagt gaacggatct cgacggtatc gcttttaaaa gaaaaggggg
2040
gattgggggg tacagtgcag gggaaagaat agtagacata atagcaacag acatacaaac
2100
taaagaatta caaaaacaaa ttacaaaaat tcaaaatttt atcgataagc tttgcaaaga
2160
tggataaagt tttaaacaga gaggaatctt tgcagctaat ggaccttcta ggtcttgaaa
2220
ggagtgggaa ttggctccgg tgcccgtcag tgggcagagc gcacatcgcc cacagtcccc
2280
gagaagttgg ggggaggggt cggcaattga accggtgcct agagaaggtg gcgcggggta
2340
aactgggaaa gtgatgtcgt gtactggctc cgcctttttc ccgagggtgg gggagaaccg
2400
tatataagtg cagtagtcgc cgtgaacgtt ctttttcgca acgggtttgc cgccagaaca
2460
caggtaagtg ccgtgtgtgg ttcccgcggg cctggcctct ttacgggtta tggcccttgc
2520
gtgccttgaa ttacttccac gcccctggct gcagtacgtg attcttgatc ccgagcttcg
2580
ggttggaagt gggtgggaga gttcgaggcc ttgcgcttaa ggagcccctt cgcctcgtgc
2640
ttgagttgag gcctggcctg ggcgctgggg ccgccgcgtg cgaatctggt ggcaccttcg
2700
cgcctgtctc gctgctttcg ataagtctct agccatttaa aatttttgat gacctgctgc
2760
gacgcttttt ttctggcaag atagtcttgt aaatgcgggc caagatctgc acactggtat
2820
ttcggttttt ggggccgcgg gcggcgacgg ggcccgtgcg tcccagcgca catgttcggc
2880
gaggcggggc ctgcgagcgc ggccaccgag aatcggacgg gggtagtctc aagctggccg
2940
gcctgctctg gtgcctggcc tcgcgccgcc gtgtatcgcc ccgccctggg cggcaaggct
3000
ggcccggtcg gcaccagttg cgtgagcgga aagatggccg cttcccggcc ctgctgcagg
3060
gagctcaaaa tggaggacgc ggcgctcggg agagcgggcg ggtgagtcac ccacacaaag
3120
gaaaagggcc tttccgtcct cagccgtcgc ttcatgtgac tccacggagt accgggcgcc
3180
gtccaggcac ctcgattagt tctcgagctt ttggagtacg tcgtctttag gttgggggga
3240
ggggttttat gcgatggagt ttccccacac tgagtgggtg gagactgaag ttaggccagc
3300
ttggcacttg atgtaattct ccttggaatt tgcccttttt gagtttggat cttggttcat
3360
tctcaagcct cagacagtgg ttcaaagttt ttttcttcca tttcaggtgt cgtgagagga
3420
attctgcagt cgagaggagc gcgcgtaata cgactcacta tagggcgcca tgggtaccgg
3480
gccccccctc gatcgaacaa caacaacaat aacacatggt tccgcgtggc tctcatatgg
3540
cggcccggcg cggggctctc atagtgctgg agggcgtgga ccgcgccggg aagagcacgc
3600
agagccgcaa gctggtggaa gcgctgtgcg ccgcgggcca ccgcgccgaa ctgctccggt
3660
tcccggaaag atcaactgaa atcggcaaac ttctgagttc ctacttgcaa aagaaaagtg
3720
acgtggagga tcactcggtg cacctgcttt tttctgcaaa tcgctgggaa caagtgccgt
3780
taattaagga aaagttgagc cagggcgtga ccctcgtcgt ggacagatac gcattttctg
3840
gtgtggccta cacaggtgcc aaggagaatt tttccctaga ctggtgtaaa cagccagacg
3900
tgggccttcc caaacccgac ctggtcctgt tcctccagtt acagctggcg gatgctgcca
3960
agcggggagc gtttggccat gagcgctatg agaacggggc tttccaggag cgggcgctcc
4020
ggtgtttcca ccagctcatg aaagacacga ctttgaactg gaagatggtg gatgcttcca
4080
aaagcatcga agctgtccat gaggacatcc gcgtgctctc tgaggacgcc atcgccactg
4140
ccacagagaa gccgctgggg gagctatgga agtgaggatc agtcgacggt atcgattccc
4200
cctctccctc ccccccccct aacgttactg gccgaagccg cttggaataa ggccggtgtg
4260
cgtttgtcta tatgttattt tccaccatat tgccgtcttt tggcaatgtg agggcccgga
4320
aacctggccc tgtcttcttg acgagcattc ctaggggtct ttcccctctc gccaaaggaa
4380
tgcaaggtct gttgaatgtc gtgaaggaag cagttcctct ggaagcttct tgaagacaaa
4440
caacgtctgt agcgaccctt tgcaggcagc ggaacccccc acctggcgac aggtgcctct
4500
gcggccaaaa gccacgtgta taagatacac ctgcaaaggc ggcacaaccc cagtgccacg
4560
ttgtgagttg gatagttgtg gaaagagtca aatggctctc ctcaagcgta ttcaacaagg
4620
ggctgaagga tgcccagaag gtaccccatt gtatgggatc tgatctgggg cctcggtgca
4680
catgctttac gtgtgtttag tcgaggttaa aaaacgtcta ggccccccga accacgggga
4740
cgtggttttc ctttgaaaaa cacgatgata tcgaattcct gcagcccggg ggatccgccc
4800
cctctgacca ccatgccacc tcctcgcctc ctcttcttcc tcctcttcct cacccccatg
4860
gaagtcaggc ccgaggaacc tctagtggtg aaggtggaag agggagataa cgctgtgctg
4920
cagtgcctca aggggacctc agatggcccc actcagcagc tgacctggtc tcgggagtcc
4980
ccgcttaaac ccttcttaaa actcagcctg gggctgccag gcctgggaat ccacatgagg
5040
cccctggcat cctggctttt catcttcaac gtctctcaac agatgggggg cttctacctg
5100
tgccagccgg ggcccccctc tgagaaggcc tggcagcctg gctggacagt caatgtggag
5160
ggcagcgggg agctgttccg gtggaatgtt tcggacctag gtggcctggg ctgtggcctg
5220
aagaacaggt cctcagaggg ccccagctcc ccttccggga agctcatgag ccccaagctg
5280
tatgtgtggg ccaaagaccg ccctgagatc tgggagggag agcctccgtg tgtcccaccg
5340
agggacagcc tgaaccagag cctcagccag gacctcacca tggcccctgg ctccacactc
5400
tggctgtcct gtggggtacc ccctgactct gtgtccaggg gccccctctc ctggacccat
5460
gtgcacccca aggggcctaa gtcattgctg agcctagagc tgaaggacga tcgcccggcc
5520
agagatatgt gggtaatgga gacgggtctg ttgttgcccc gggccacagc tcaagacgct
5580
ggaaagtatt attgtcaccg tggcaacctg accatgtcat tccacctgga gatcactgct
5640
cggccagtac tatggcactg gctgctgagg actggtggct ggaaggtctc agctgtgact
5700
ttggcttatc tgatcttctg cctgtgttcc cttgtgggca ttcttcatct ttaaggcgcg
5760
ccccgggatc caagcttcaa ttgtggtcac tcgacaatca acctctggat tacaaaattt
5820
gtgaaagatt gactggtatt cttaactatg ttgctccttt tacgctatgt ggatacgctg
5880
ctttaatgcc tttgtatcat gctattgctt cccgtatggc tttcattttc tcctccttgt
5940
ataaatcctg gttgctgtct ctttatgagg agttgtggcc cgttgtcagg caacgtggcg
6000
tggtgtgcac tgtgtttgct gacgcaaccc ccactggttg gggcattgcc accacctgtc
6060
agctcctttc cgggactttc gctttccccc tccctattgc cacggcggaa ctcatcgccg
6120
cctgccttgc ccgctgctgg acaggggctc ggctgttggg cactgacaat tccgtggtgt
6180
tgtcggggaa gctgacgtcc tttccatggc tgctcgcctg tgttgccacc tggattctgc
6240
gcgggacgtc cttctgctac gtcccttcgg ccctcaatcc agcggacctt ccttcccgcg
6300
gcctgctgcc ggctctgcgg cctcttccgc gtcttcgcct tcgccctcag acgagtcgga
6360
tctccctttg ggccgcctcc ccgcctgtct cgagacctag aaaaacatgg agcaatcaca
6420
agtagcaata cagcagctac caatgctgat tgtgcctggc tagaagcaca agaggaggag
6480
gaggtgggtt ttccagtcac acctcaggta cctttaagac caatgactta caaggcagat
6540
cttagccact ttttaaaaga aaagggggga ctggaagggc taattcactc ccaacgaaga
6600
caagatctgc tttttgcttg tactgggtct ctctggttag accagatctg agcctgggag
6660
ctctctggct aactagggaa cccactgctt aagcctcaat aaagcttgcc ttgagtgctt
6720
caagtagtgt gtgcccgtct gttgtgtgac tctggtaact agagatccct cagacccttt
6780
tagtcagtgt ggaaaatctc tagca
6805
<210> 15
<211> 638
<212> DNA
<213> Homo sapiens
<400> 15
tggcggcccg gcgcggggct ctcatagtgc tggagggcgt ggaccgcgcc gggaagagca
60
cgcagagccg caagctggtg gaagcgctgt gcgccgcggg ccaccgcgcc gaactgctcc
120
ggttcccgga aagatcaact gaaatcggca aacttctgag ttcctacttg caaaagaaaa
180
gtgacgtgga ggatcactcg gtgcacctgc ttttttctgc aaatcgctgg gaacaagtgc
240
cgttaattaa ggaaaagttg agccagggcg tgaccctcgt cgtggacaga tacgcatttt
300
ctggtgtggc cttcacaggt gccaaggaga atttttccct agactggtgt aaacagccag
360
acgtgggcct tcccaaaccc gacctggtcc tgttcctcca gttacagctg gcggatgctg
420
ccaagcgggg agcgtttggc catgagcgct atgagaacgg ggctttccag gagcgggcgc
480
tccggtgttt ccaccagctc atgaaagaca cgactttgaa ctggaagatg gtggatgctt
540
ccaaaagcat cgaagctgtc catgaggaca tccgcgtgct ctctgaggac gccatccgca
600
ctgccacaga gaagccgctg ggggagctat ggaagtga
638
<210> 16
<211> 211
<212> PRT
<213> Homo sapiens
<400> 16
Met Ala Ala Arg Arg Gly Ala Leu Ile Val Leu Glu Gly Val Asp Arg
1 5 10 15
Ala Gly Lys Ser Thr Gln Ser Arg Lys Leu Val Glu Ala Leu Cys Ala
                        20 25 30
Ala Gly His Arg Ala Glu Leu Leu Arg Phe Pro Glu Arg Ser Thr Glu
                35 40 45
Ile Gly Lys Leu Leu Ser Ser Tyr Gln Lys Lys Ser Asp Val Glu Asp
        50 55 60
His Ser Val His Leu Leu Phe Ser Ala Asn Arg Trp Glu Gln Val Pro
65 70 75 80
Leu Ile Lys Glu Lys Leu Ser Gln Gly Val Thr Leu Val Val Asp Arg
                                85 90 95
Tyr Ala Phe Ser Gly Val Ala Phe Thr Gly Ala Lys Glu Asn Phe Ser
                        100 105 110
Leu Asp Trp Cys Lys Gln Pro Asp Val Gly Leu Pro Lys Pro Asp Leu
                115 120 125
Val Leu Phe Leu Gln Leu Gln Leu Ala Asp Ala Ala Lys Arg Gly Ala
        130 135 140
Phe Gly His Glu Arg Tyr Glu Asn Gly Ala Phe Gln Glu Arg Ala Leu
145 150 155 160
Arg Cys Phe His Gln Leu Met Lys Asp Thr Thr Leu Asn Trp Lys Met
                                165 170 175
Val Asp Ala Ser Lys Ser Ile Glu Ala Val His Glu Asp Ile Arg Val
                        180 185 190
Leu Ser Glu Asp Ala Ile Ala Thr Ala Thr Glu Lys Pro Leu Gly Glu
                195 200 205
Leu Trp Lys
        210
<210> 17
<211> 15
<212> PRT
<213> E. coli
<400> 17
Thr Pro Glu Val Gly Leu Lys Arg Ala Arg Ala Arg Gly Glu Leu
1 5 10 15
<210> 18
<211> 118
<212> DNA
<213> HIV
<400> 18
ttttaaaaga aaagggggga ttggggggta cagtgcaggg gaaagaatag tagacataat
60
agcaacagac atacaaacta aagaattaca aaaacaaatt acaaaaattc aaaatttt
118
<210> 19
<211> 592
<212> DNA
<213> Woodchuck Hepatitus Virus
<400> 19
aatcaacctc tggattacaa aatttgtgaa agattgactg gtattcttaa ctatgttgct
60
ccttttacgc tatgtggata cgctgcttta atgcctttgt atcatgctat tgcttcccgt
120
atggctttca ttttctcctc cttgtataaa tcctggttgc tgtctcttta tgaggagttg
180
tggcccgttg tcaggcaacg tggcgtggtg tgcactgtgt ttgctgacgc aacccccact
240
ggttggggca ttgccaccac ctgtcagctc ctttccggga ctttcgcttt ccccctccct
300
attgccacgg cggaactcat cgccgcctgc cttgcccgct gctggacagg ggctcggctg
360
ttgggcactg acaattccgt ggtgttgtcg gggaagctga cgtcctttcc atggctgctc
420
gcctgtgttg ccacctggat tctgcgcggg acgtccttct gctacgtccc ttcggccctc
480
aatccagcgg accttccttc ccgcggcctg ctgccggctc tgcggcctct tccgcgtctt
540
cgccttcgcc ctcagacgag tcggatctcc ctttgggccg cctccccgcc tg
592
<210> 20
<211> 13
<212> PRT
<213> Homo sapiens
<400> 20
Gln Leu Ala Asp Ala Ala Lys Arg Gly Ala Phe Gly His
1 5 10
<210> 21
<211> 639
<212> DNA
<213> Homo sapiens
<400> 21
atggcggccc ggcgcggggc tctcatagtg ctggagggcg tggaccgcgc cgggaagagc
60
acgcagagcc gcaagctggt ggaagcgctg tgcgccgcgg gccaccgcgc cgaactgctc
120
cggttcccgg aaagatcaac tgaaatcggc aaacttctga gttcctactt gcaaaagaaa
180
agtgacgtgg aggatcactc ggtgcacctg cttttttctg caaatcgctg ggaacaagtg
240
ccgttaatta aggaaaagtt gagccagggc gtgaccctcg tcgtggacag atac˜cattt
300
tctggtgtgg cctacacagg tgccaaggag aatttttccc tagactggtg taaacagcca
360
gacgtgggcc ttcccaaacc cgacctggtc ctgttcctcc agttacagct ggcggatgct
420
gccaagcggg gagcgtttgg ccatgagcgc tatgagaacg gggctttcca ggagcgggcg
480
ctccggtgtt tccaccagct catgaaagac acgactttga actggaagat ggtggatgct
540
tccaaaagca tcgaagctgt ccatgaggac atccgcgtgc tctctgagga cgccatcgcc
600
actgccacag agaagccgct gggggagcta tggaagtga
639
<210> 22
<211> 645
<212> DNA
<213> Homo sapiens
<400> 22
atggcggccc ggcgcggggc tctcatagtg ctggagggcg tggacggcgc cgggaagagc
60
acgcagagcc gcaagctggt ggaagcgctg tgcgccgcgg gccaccgcgc cgaactgctc
120
cggttcccgg aaagatcaac tgaaatcggc aaacttctga gttcctactt gcaaaagaaa
180
agtgacgtgg aggatcactc ggtgcacctg cttttttctg caaatcgctg ggaacaagtg
240
ccgttaatta aggaaaagtt gagccagggc gtgaccctcg tcgtggacag atacgcattt
300
tctggtgtgg ccttcaccgg tgccaaggag aatttttccc tagactggtg taaacagcca
360
gacgtgggcc ttcccaaacc cgacctggtc ctgttcctgc agttaactcc ggaagttggc
420
ttaaaacgcg cacgtgctcg cggcgagctt gaccgctatg agaacggggc tttccaggag
480
cgggcgctcc ggtgtttcca ccagctcatg aaagacacga ctttgaactg gaagatggtg
540
gatgcttcca aaagcatcga agctgtccat gaggacatcc gcgtgctctc tgaggacgcc
600
atcgccactg ccacagagaa gccgctgggg gagctatgga agtga
645
<210> 23
<211> 28
<212> DNA
<213> Homo sapiens
<400> 23
atgccacctc ctcgcctcct cttcttcc
28
<210> 24
<211> 22
<212> DNA
<213> Homo sapiens
<400> 24
tcacctggtg ctccaggtgc cc
22
<210> 25
<211> 23
<212> DNA
<213> Homo sapiens
<400> 25
ccgccaccgc ggtggagctc cag
23
<210> 26
<211> 26
<212> DNA
<213> Homo sapiens
<400> 26
ttaaagatga agaatgccca caaggg
26
<210> 27
<211> 1966
<212> DNA
<213> Homo sapiens
<400> 27
aggcccctgc ctgccccagc atcccctgcg cgaagctggg tgccccggag agtctgacca
60
ccatgccacc tcctcgcctc ctcttcttcc tcctcttcct cacccccatg gaagtcaggc
120
ccgaggaacc tctagtggtg aaggtggaag aggagataa cgctgtgctg cagtgcctca
180
aggggacctc agatggcccc actcagcagc tgacctggtc tcgggagtcc ccgcttaaac
240
ccttcttaaa actcagcctg gggctgccag gcctgggaat ccacatgagg cccctggcca
300
tctggctttt catcttcaac gtctctcaac agatgggggg cttctacctg tgccagccgg
360
ggcccccctc tgagaaggcc tggcagcctg gctggacagt caatgtggag ggcagcgggg
420
agctgttccg gtggaatgtt tcggacctag gtggcctggg ctgtggcctg aagaacaggt
480
cctcagaggg ccccagctcc ccttccggga agctcatgag ccccaagctg tatgtgtggg
540
ccaaagaccg ccctgagatc tgggagggag agcctccgtg tctcccaccg agggacagcc
600
tgaaccagag cctcagccag gacctcacca tggcccctgg ctccacactc tggctgtcct
660
gtggggtacc ccctgactct gtgtccaggg gccccctctc ctggacccat gtgcacccca
720
aggggcctaa gtcattgctg agcctagagc tgaaggacga tcgcccggcc agagatatgt
780
gggtaatgga gacgggtctg ttgttgcccc gggccacagc tcaagacgct ggaaagtatt
840
attgtcaccg tggcaacctg accatgtcat tccacctgga gatcactgct cggccagtac
900
tatggcactg gctgctgagg actggtggct ggaaggtctc agctgtgact ttggcttatc
960
tgatcttctg cctgtgttcc cttgtgggca ttcttcatct tcaaagagcc ctggtcctga
1020
ggaggaaaag aaagcgaatg actgacccca ccaggagatt cttcaaagtg acgcctcccc
1080
caggaagcgg gccccagaac cagtacggga acgtgctgtc tctccccaca cccacctcag
1140
gcctcggacg cgcccagcgt tgggccgcag gcctgggggg cactgccccg tcttatggaa
1200
acccgagcag cgacgtccag gcggatggag ccttggggtc ccggagcccg ccgggagtgg
1260
gcccagaaga agaggaaggg gagggctatg aggaacctga cagtgaggag gactccgagt
1320
tctatgagaa cgactccaac cttgggcagg accagctctc ccaggatggc agcggctacg
1380
agaaccctga ggatgagccc ctgggtcctg aggatgaaga ctccttctcc aacgctgagt
1440
cttatgagaa cgaggatgaa gagctgaccc agccggtcgc caggacaatg gacttcctga
1500
gccctcatgg gtcagcctgg gaccccagcc gggaagcaac ctccctgggg tcccagtcct
1560
atgaggatat gagaggaatc ctgtatgcag ccccccagct ccgctccatt cggggccagc
1620
ctggacccaa tcatgaggaa gatgcagact cttatgagaa catggataat cccgatgggc
1680
cagacccagc ctggggagga gggggccgca tgggcacctg gagcaccagg tgatcctcag
1740
gtggccagcc tggatctcct caagtcccca agattcacac ctgactctga aatctgaaga
1800
cctcgagcag atgatgccaa cctctggagc aatgttgctt aggatgtgtg catgtgtgta
1860
agtgtgtgtg tgtgtgtgtg tgtgtataca tgccagtgac acttccagtc ccctttgtat
1920
tccttaaata aactcaatga gctcttccaa aaaaaaaaaa aaaaaa
1966
<210> 28
<211> 556
<212> PRT
<213> Homo sapiens
<400> 28
Met Pro Pro Pro Arg Leu Leu Phe Phe Leu Leu Phe Leu Thr Pro Met
1 5 10 15
Glu Val Arg Pro Glu Glu Pro Leu Val Val Lys Val Glu Glu Gly Asp
                        20 25 30
Asn Ala Val Leu Gln Cys Leu Lys Gly Thr Ser Asp Gly Pro Thr Gln
                35 40 45
Gln Leu Thr Trp Ser Arg Glu Ser Pro Leu Lys Pro Phe Leu Lys Leu
        50 55 60
Ser Leu Gly Leu Pro Gly Leu Gly Ile His Met Arg Pro Leu Ala Ile
65 70 75 80
Trp Leu Phe Ile Phe Asn Val Ser Gln Gln Met Gly Gly Phe Tyr Leu
                                85 90 95
Cys Gln Pro Gly Pro Pro Ser Glu Lys Ala Trp Gln Pro Gly Trp Thr
                        100 105 110
Val Asn Val Glu Gly Ser Gly Glu Leu Phe Arg Trp Asn Val Ser Asp
                115 120 125
Leu Gly Gly Leu Gly Cys Gly Leu Lys Asn Arg Ser Ser Glu Gly Pro
        130 135 140
Ser Ser Pro Ser Gly Lys Leu Met Ser Pro Lys Leu Tyr Val Trp Ala
145 150 155 160
Lys Asp Arg Pro Glu Ile Trp Glu Gly Glu Pro Pro Cys Leu Pro Pro
                                165 170 175
Arg Asp Ser Leu Asn Gln Ser Leu Ser Gln Asp Leu Thr Met Ala Pro
                        180 185 190
Gly Ser Thr Leu Trp Leu Ser Cys Gly Val Pro Pro Asp Ser Val Ser
                195 200 205
Arg Gly Pro Leu Ser Trp Thr His Val His Pro Lys Gly Pro Lys Ser
        210 215 220
Leu Leu Ser Leu Glu Leu Lys Asp Asp Arg Pro Ala Arg Asp Met Trp
225 230 235 240
Val Met Glu Thr Gly Leu Leu Leu Pro Arg Ala Thr Ala Gln Asp Ala
                                245 250 255
Gly Lys Tyr Tyr Cys His Arg Gly Asn Leu Thr Met Ser Phe His Leu
                        260 265 270
Glu Ile Thr Ala Arg Pro Val Leu Trp His Trp Leu Leu Arg Thr Gly
                275 280 285
Gly Trp Lys Val Ser Ala Val Thr Leu Ala Tyr Leu Ile Phe Cys Leu
        290 295 300
Cys Ser Leu Val Gly Ile Leu His Leu Gln Arg Ala Leu Val Leu Arg
305 310 315 320
Arg Lys Arg Lys Arg Met Thr Asp Pro Thr Arg Arg Phe Phe Lys Val
                                325 330 335
Thr Pro Pro Pro Gly Ser Gly Pro Gln Asn Gln Tyr Gly Asn Val Leu
                        340 345 350
Ser Leu Pro Thr Pro Thr Ser Gly Leu Gly Arg Ala Gln Arg Trp Ala
                355 360 365
Ala Gly Leu Gly Gly Thr Ala Pro Ser Tyr Gly Asn Pro Ser Ser Asp
        370 375 380
Val Gln Ala Asp Gly Ala Leu Gly Ser Arg Ser Pro Pro Gly Val Gly
385 390 395 400
Pro Glu Glu Glu Glu Gly Glu Gly Tyr Glu Glu Pro Asp Ser Glu Glu
                                405 410 415
Asp Ser Glu Phe Tyr Glu Asn Asp Ser Asn Leu Gly Gln Asp Gln Leu
                        420 425 430
Ser Gln Asp Gly Ser Gly Tyr Glu Asn Pro Glu Asp Glu Pro Leu Gly
                435 440 445
Pro Glu Asp Glu Asp Ser Phe Ser Asn Ala Glu Ser Tyr Glu Asn Glu
        450 455 460
Asp Glu Glu Leu Thr Gln Pro Val Ala Arg Thr Met Asp Phe Leu Ser
465 470 475 480
Pro His Gly Ser Ala Trp Asp Pro Ser Arg Glu Ala Thr Ser Leu Gly
                                485 490 495
Ser Gln Ser Tyr Glu Asp Met Arg Gly Ile Leu Tyr Ala Ala Pro Gln
                        500 505 510
Leu Arg Ser Ile Arg Gly Gln Pro Gly Pro Asn His Glu Glu Asp Ala
                515 520 525
Asp Ser Tyr Glu Asn Met Asp Asn Pro Asp Gly Pro Asp Pro Ala Trp
        530 535 540
Gly Gly Gly Gly Arg Met Gly Thr Trp Ser Thr Arg
545 550 555
<210> 29
<211> 313
<212> PRT
<213> Homo sapiens
<400> 29
Met Pro Pro Pro Arg Leu Leu Phe Phe Leu Leu Phe Leu Thr Pro Met
1 5 10 15
Glu Val Arg Pro Glu Glu Pro Leu Val Val Lys Val Glu Glu Gly Asp
                        20 25 30
Asn Ala Val Leu Gln Cys Leu Lys Gly Thr Ser Asp Gly Pro Thr Gln
                35 40 45
Gln Leu Thr Trp Ser Arg Glu Ser Pro Leu Lys Pro Phe Leu Lys Leu
        50 55 60
Ser Leu Gly Leu Pro Gly Leu Gly Ile His Met Arg Pro Leu Ala Ile
65 70 75 80
Trp Leu Phe Ile Phe Asn Val Ser Gln Gln Met Gly Gly Phe Tyr Leu
                                85 90 95
Cys Gln Pro Gly Pro Pro Ser Glu Lys Ala Trp Gln Pro Gly Trp Thr
                        100 105 110
Val Asn Val Glu Gly Ser Gly Glu Leu Phe Arg Trp Asn Val Ser Asp
                115 120 125
Leu Gly Gly Leu Gly Cys Gly Leu Lys Asn Arg Ser Ser Glu Gly Pro
        130 135 140
Ser Ser Pro Ser Gly Lys Leu Met Ser Pro Lys Leu Tyr Val Trp Ala
145 150 155 160
Lys Asp Arg Pro Glu Ile Trp Glu Gly Glu Pro Pro Cys Leu Pro Pro
                                165 170 175
Arg Asp Ser Leu Asn Gln Ser Leu Ser Gln Asp Leu Thr Met Ala Pro
                        180 185 190
Gly Ser Thr Leu Trp Leu Ser Cys Gly Val Pro Pro Asp Ser Val Ser
                195 200 205
Arg Gly Pro Leu Ser Trp Thr His Val His Pro Lys Gly Pro Lys Ser
        210 215 220
Leu Leu Ser Leu Glu Leu Lys Asp Asp Arg Pro Ala Arg Asp Met Trp
225 230 235 240
Val Met Glu Thr Gly Leu Leu Leu Pro Arg Ala Thr Ala Gln Asp Ala
                                245 250 255
Gly Lys Tyr Tyr Cys His Arg Gly Asn Leu Thr Met Ser Phe His Leu
                        260 265 270
Glu Ile Thr Ala Arg Pro Val Leu Trp His Trp Leu Leu Arg Thr Gly
                275 280 285
Gly Trp Lys Val Ser Ala Val Thr Leu Ala Tyr Leu Ile Phe Cys Leu
        290 295 300
Cys Ser Leu Val Gly Ile Leu His Leu
305 310
<210> 30
<211> 939
<212> DNA
<213> Homo sapiens
<220>
<221> exon
<222> (1) . . . (939)
<400> 30
atg cca cct cct cgc ctc ctc ttc ttc ctc atc ttc ctc acc ccc atg
48
Met Pro Pro Pro Arg Leu Leu Phe Phe Leu Leu Phe Leu Thr Pro Met
1 5 10 15
gaa gtc agg ccc gag gaa cct cta gtg gtg aag gtg gaa gag gga gat
96
Glu Val Arg Pro Glu Glu Pro Leu Val Val Lys Val Glu Glu Gly Asp
                        20 25 30
aac gct gtg ctg cag tgc ctc aag ggg acc tca gat ggc ccc act cag
144
Asn Ala Val Leu Gln Cys Leu Lys Gly Thr Ser Asp Gly Pro Thr Gln
                35 40 45
cag ctg acc tgg tct cgg gag tcc ccg ctt aaa ccc ttc tta aaa ctc
192
Gln Leu Thr Trp Ser Arg Glu Ser Pro Leu Lys Pro Phe Leu Lys Leu
        50 55 60
agc ctg ggg ctg cca ggc ctg gga atc cac atg agg ccc ctg gcc atc
240
Ser Leu Gly Leu Pro Gly Leu Gly Ile His Met Arg Pro Leu Ala Ile
65 70 75 80
tgg ctt ttc atc ttc aac gtc tct caa cag atg ggg ggc ttc tac ctg
288
Trp Leu Phe Ile Phe Asn Val Ser Gln Gln Met Gly Gly Phe Tyr Leu
                                85 90 95
tgc cag ccg ggg ccc ccc tct gag aag gcc tgg cag cct ggc tgg aca
336
Cys Gln Pro Gly Pro Pro Ser Glu Lys Ala Trp Gln Pro Gly Trp Thr
                        100 105 110
gtc aat gtg gag ggc agc ggg gag ctg ttc cgg tgg aat gtt tcg gac
384
Val Asn Val Glu Gly Ser Gly Glu Leu Phe Arg Trp Asn Val Ser Asp
                115 120 125
cta ggt ggc ctg ggc tgt ggc ctg aag aac agg tcc tca gag ggc ccc
432
Leu Gly Gly Leu Gly Cys Gly Leu Lys Asn Arg Ser Ser Glu Gly Pro
        130 135 140
agc tcc cct tcc ggg aag ctc atg agc ccc aag ctg tat gtg tgg gcc
480
Ser Ser Pro Ser Gly Lys Leu Met Ser Pro Lys Leu Tyr Val Trp Ala
145 150 155 160
aaa gac cgc cct gag atc tgg gag gga gag cct ccg tgt ctc cca ccg
528
Lys Asp Arg Pro Glu Ile Trp Glu Gly Glu Pro Pro Cys Leu Pro Pro
                                165 170 175
agg gac agc ctg aac cag agc ctc agc cag gac ctc acc atg gcc cct
576
Arg Asp Ser Leu Asn Gln Ser Leu Ser Gln Asp Leu Thr Met Ala Pro
                        180 185 190
ggc tcc aca ctc tgg ctg tcc tgt ggg gta ccc cct gac tct gtg tcc
624
Gly Ser Thr Leu Trp Leu Ser Cys Gly Val Pro Pro Asp Ser Val Ser
                195 200 205
agg ggc ccc ctc tcc tgg acc cat gtg cac ccc aag ggg cct aag tca
672
Arg Gly Pro Leu Ser Trp Thr His Val His Pro Lys Gly Pro Lys Ser
        210 215 220
ttg ctg agc cta gag ctg aag gac gat cgc ccg gcc aga gat atg tgg
720
Leu Leu Ser Leu Glu Leu Lys Asp Asp Arg Pro Ala Arg Asp Met Trp
225 230 235 240
gta atg gag acg ggt ctg ttg ttg ccc cgg gcc aca gct caa gac gct
768
Val Met Glu Thr Gly Leu Leu Leu Pro Arg Ala Thr Ala Gln Asp Ala
                                245 250 255
gga aag tat tat tgt cac cgt ggt aac ctg acc atg tca ttc cac ctg
816
Gly Lys Tyr Tyr Cys His Arg Gly Asn Leu Thr Met Ser Phe His Leu
                        260 265 270
gag atc act gct cgg cca gta cta tgg cac tgg ctg ctg agg act ggt
864
Glu Ile Thr Ala Arg Pro Val Leu Trp His Trp Leu Leu Arg Thr Gly
                275 280 285
ggc tgg aag gtc tca gct gtg act ttg gct tat ctg atc ttc tgc ctg
912
Gly Trp Lys Val Ser Ala Val Thr Leu Ala Tyr Leu Ile Phe Cys Leu
        290 295 300
tgt tcc ctt gtg ggc att ctt cat ctt
939
Cys Ser Leu Val Gly Ile Leu His Leu
305 310
<210> 31
<211> 313
<212> PRT
<213> Homo sapiens
<400> 31
Met Pro Pro Pro Arg Leu Leu Phe Phe Leu Leu Phe Leu Thr Pro Met
1 5 10 15
Glu Val Arg Pro Glu Glu Pro Leu Val Val Lys Val Glu Glu Gly Asp
                        20 25 30
Asn Ala Val Leu Gln Cys Leu Lys Gly Thr Ser Asp Gly Pro Thr Gln
                35 40 45
Gln Leu Thr Trp Ser Arg Glu Ser Pro Leu Lys Pro Phe Leu Lys Leu
        50 55 60
Ser Leu Gly Leu Pro Gly Leu Gly Ile His Met Arg Pro Leu Ala Ser
65 70 75 80
Trp Leu Phe Ile Phe Asn Val Ser Gln Gln Met Gly Gly Phe Tyr Leu
                                85 90 95
Cys Gln Pro Gly Pro Pro Ser Glu Lys Ala Trp Gln Pro Gly Trp Thr
                        100 105 110
Val Asn Val Glu Gly Ser Gly Glu Leu Phe Arg Trp Asn Val Ser Asp
                115 120 125
Leu Gly Gly Leu Gly Cys Gly Leu Lys Asn Arg Ser Ser Glu Gly Pro
        130 135 140
Ser Ser Pro Ser Gly Lys Leu Met Ser Pro Lys Leu Tyr Val Trp Ala
145 150 155 160
Lys Asp Arg Pro Glu Ile Trp Glu Gly Glu Pro Pro Cys Val Pro Pro
                                165 170 175
Arg Asp Ser Leu Asn Gln Ser Leu Ser Gln Asp Leu Thr Met Ala Pro
                        180 185 190
Gly Ser Thr Leu Trp Leu Ser Cys Gly Val Pro Pro Asp Ser Val Ser
                195 200 205
Arg Gly Pro Leu Ser Trp Thr His Val His Pro Lys Gly Pro Lys Ser
        210 215 220
Leu Leu Ser Leu Glu Leu Lys Asp Asp Arg Pro Ala Arg Asp Met Trp
225 230 235 240
Val Met Glu Thr Gly Leu Leu Leu Pro Arg Ala Thr Ala Gln Asp Ala
                                245 250 255
Gly Lys Tyr Tyr Cys His Arg Gly Asn Leu Thr Met Ser Phe His Leu
                        260 265 270
Glu Ile Thr Ala Arg Pro Val Leu Trp His Trp Leu Leu Arg Thr Gly
                275 280 285
Gly Trp Lys Val Ser Ala Val Thr Leu Ala Tyr Leu Ile Phe Cys Leu
        290 295 300
Cys Ser Leu Val Gly Ile Leu His Leu
305 310
```

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

All publications, patents and patent applications, are herein incorporated by reference in their entirety to the same extent as if each individual publication, patent or patent application was specifically and individually indicated to be incorporated by reference in its entirety.

### REFERENCES

1. Hacein-Bey-Abina S, Von Kalle C, Schmidt M, McCormack M P, Wulffraat N, Leboulch P, et al. LMO2-associated clonal T cell proliferation in two patients after gene therapy for SCID-X1. Science. 2003; 302: 415-419.
2. Roy N S, Cleren C, Singh S K, Yang L, Beal M F, Goldman S A. Functional engraftment of human ES cell-derived dopaminergic neurons enriched by coculture with telomerase-immortalized midbrain astrocytes. Nat. Med. 2006; published online: 22 Oct. 2006.
3. Nishiyama Y, Rapp F. Anticellular effects of 9-(2-hydroxyethoxymethyl) guanine against herpes simplex virus-transformed cells. J Gen Virol. 1979; 45: 227-230.
4. Moolten F L. Tumor chemosensitivity conferred by inserted herpes thymidine kinase genes: paradigm for a prospective cancer control strategy. Cancer Res. 1986; 46: 5276-5281.
5. Wildner O, Blaese R M, Morris J C. Therapy of colon cancer with oncolytic adenovirus is enhanced by the addition of herpes simplex virus-thymidine kinase. Cancer Res. 1999; 59: 410-413.
6. Moolten F L, Wells J M. Curability of tumors bearing herpes thymidine kinase genes transferred by retroviral vectors. J Natl Cancer Inst. 1990; 82: 297-300.
7. Hamel W, Magnelli L, Chiarugi V P, Israel M A. Herpes simplex virus thymidine kinase/ganciclovir-mediated apoptotic death of bystander cells. Cancer Res. 1996; 56: 2697-2702.
8. Kokoris M S, Black M E. Characterization of herpes simplex virus type 1 thymidine kinase mutants engineered for improved ganciclovir or acyclovir activity. Protein Sci. 2002; 11: 2267-2272.
9. Qasim W, Thrasher A J, Buddle J, Kinnon C, Black M E, Gaspar H B. T cell transduction and suicide with an enhanced mutant thymidine kinase. Gene Ther. 2002; 9: 824-827.
10. Riddell S R, Elliott M, Lewinsohn D A, Gilbert M J, Wilson L, Manley S A, et al. T-cell mediated rejection of gene-modified HIV-specific cytotoxic T lymphocytes in HIV-infected patients. Nat Med. 1996; 2: 216-223.
11. Berger C, Flowers M E, Warren E H, Riddell S R. Analysis of transgene-specific immune responses that limit the in vivo persistence of adoptively transferred HSV-TK-modified donor T cells after allogeneic hematopoietic cell transplantation. Blood. 2006; 107: 2294-2302.
12. Van Rompay A R, Johansson M, Karlsson A. Phosphorylation of nucleosides and nucleoside analogs by mammalian nucleoside monophosphate kinases. Pharmacol Ther 2000; 87: 189-198.
13. Furman P A, Fyfe J A, St Clair M H, Weinhold K, Rideout J L, Freeman G A, et al. Phosphorylation of 3′-azido-3′-deoxythymidine and selective interaction of the 5′-triphosphate with human immunodeficiency virus reverse transcriptase. Proc Nat Acad Sci USA. 1986; 83: 8333-8337.
14. St Clair M H, Richards C A, Spector T, Weinhold K J, Miller W H, Langlois A J, et al. 3′-Azido-3′-deoxythymidine triphosphate as an inhibitor and substrate of purified human immunodeficiency virus reverse transcriptase. Antimicrob Agents Chemother. 1987; 31: 1972-1977.
15. Frick L W, Nelson D J, St Clair M H, Furman P A, Krenitsky T A. Effects of 3′-azido-3′-deoxythymidine on the deoxynucleotide triphosphate pools of cultured human cells. Biochem Biophys Res Commun. 1988; 154: 124-129.
16. Johnson A A, Ray A S, Hanes J, Suo Z, Colacino J M, Anderson K S, et al. Toxicity of antiviral nucleoside analogs and the human mitochondrial DNA polymerase. J Biol Chem. 2001; 276: 40847-40857.
17. Lavie A, Schlichting I, Vetter I R, Konrad M, Reinstein J, Goody R S. The bottleneck in AZT activation. Nat Med. 1997; 3: 922-924.
18. Coplan N L, Bruno M S. Acquired immunodeficiency syndrome and heart disease: the present and the future. Am Heart J. 1989; 117: 1175-1177.
19. Cazzalini O, Lazze M C, Iamele L, Stivala L A, Bianchi L, Vaghi P, et al. Early effects of AZT on mitochondrial functions in the absence of mitochondrial DNA depletion in rat myotubes. Biochem Pharmacol. 2001; 62: 893-902.
20. Sales S D, Hoggard P G, Sunderland D, Khoo S, Hart C A, Back D J. Zidovudine phosphorylation and mitochondrial toxicity in vitro. Toxicol Appl Pharmacol. 2001; 177: 54-58.
21. Masini A, Scotti C, Calligaro A, Cazzalini O, Stivala L A, Bianchi L, et al. Zidovudine-induced experimental myopathy: dual mechanism of mitochondrial damage. J Neurol Sci. 1999; 166: 131-140.
22. McKee E E, Bentley A T, Hatch M, Gingerich J, Susan-Resiga D. Phosphorylation of thymidine and AZT in heart mitochondria: elucidation of a novel mechanism of AZT cardiotoxicity. Cardiovasc Toxicol. 2004; 4: 155-167.
23. Brundiers R, Lavie A, Veit T, Reinstein J, Schlichting I, Ostermann N, et al. Modifying human thymidylate kinase to potentiate azidothymidine activation. J Biol Chem. 1999; 274: 35289-35292.
24. Ostermann N, Lavie A, Padiyar S, Brundiers R, Veit T, Reinstein J, et al. Potentiating AZT activation: structures of wild-type and mutant human thymidylate kinase suggest reasons for the mutants' improved kinetics with the HIV prodrug metabolite AZTMP. J Mol Biol. 2000; 304: 43-53.
25. Naldini L, Blomer U, Gallay P, Ory D, Mulligan R, Gage F H, et al. In vivo gene delivery and stable transduction of nondividing cells by a lentiviral vector. Science. 1996; 272: 263-267.
26. Blomer U, Naldini L, Kafri T, Trono D, Verma I M, Gage F H. Highly efficient and sustained gene transfer in adult neurons with a lentivirus vector. J Virol. 1997; 71: 6641-6649.
27. Yoshimitsu M, Sato T, Tao K, Walia J S, Rasaiah V I, Sleep G T, et al. Bioluminescent imaging of a marking transgene and correction of Fabry mice by neonatal injection of recombinant lentiviral vectors. Proc Nat Acad Sci USA. 2004; 101: 16909-16914.
28. Sadelain M, Riviere I. Sturm und drang over suicidal lymphocytes. Mol Ther. 2002; 5: 655-657.
29. Migita M, Medin J A, Pawliuk R, Jacobson S, Nagle J W, Anderson S, et al. Selection of transduced CD34+ progenitors and enzymatic correction of cells from Gaucher patients, with bicistronic vectors. Proc Natl Acad Sci USA. 1995; 92: 12075-12079.
30. Medin J A, Migita M, Pawliuk R, Jacobson S, Amiri M, Kluepfel-Stahl S, et al. A bicistronic therapeutic retroviral vector enables sorting of transduced CD34+ cells and corrects the enzyme deficiency in cells from Gaucher patients. Blood. 1996; 87: 1754-1762.
31. Qin G, Takenaka T, Telsch K, Kelley L, Howard T, Levade T, et al. Preselective gene therapy for Fabry disease. Proc Natl Acad Sci USA. 2001; 98: 3428-3433.
32. Siatskas C, Underwood J, Ramenazi A, Hawley R G, Medin, J. A.: Specific pharmacological dimerization of KDR in lentivirally transduced human hematopoietic cells activates anti-apoptotic and proliferative effects. FASEB J. 2005; 19: 1752-1754.
33. Medin J A, Liang S B, Hou J W, Kelley L S, Peace D J, Fowler D H. Efficient transfer of PSA and PSMA cDNAs into DCs generates antibody and T cell antitumor responses in vivo. Cancer Gene Ther. 2005; 12: 540-551.
34. Bonini C, Ferrari G, Verzeletti S, Servida P, Zappone E, Ruggieri L, et al. HSV-TK gene transfer into donor lymphocytes for control of allogeneic graft-versus-leukemia. Science. 1997; 276: 1719-1724.
35. Li Z, Dullmann J, Schiedlmeier B, Schmidt M, von Kalle C, Meyer J, et al. Murine leukemia induced by retroviral gene marking. Science. 2002; 296: 497.
36. Doody G M, Dempsey P W, Fearon D T. Activation of B lymphocytes: integrating signals from CD19, CD22 and Fc gamma RIIb-1. Curr Opin Immunol. 1996; 8: 378-382.
37. Fujimoto M, Poe J C, Hasegawa M, Tedder T F. CD19 regulates intrinsic B lymphocyte signal transduction and activation through a novel mechanism of processive amplification. Immunol Res. 2000; 22: 281-298.
38. Tedder T F, Zhou L J, Engel P. The CD19/CD21 signal transduction complex of B lymphocytes. Immunol Today. 1994; 15: 437-442.
39. Sato S, Miller A S, Howard M C, Tedder T F. Regulation of B lymphocyte development and activation by the CD19/CD21/CD81/Leu 13 complex requires the cytoplasmic domain of CD19. J Immunol. 1997; 159: 3278-3287.
40. Greco O, Dachs G U. Gene directed enzyme/prodrug therapy of cancer: historical appraisal and future prospectives. J Cell Physiol. 2001; 187: 22-36.
41. Smiley S T, Reers M, Mottola-Hartshorn C, Lin M, Chen A, Smith T W, et al. Intracellular heterogeneity in mitochondrial membrane potentials revealed by a J-aggregate-forming lipophilic cation JC-1. Proc Nat Acad Sci USA. 1991; 88: 3671-3675.
42. Green D R, Reed J C. Mitochondria and apoptosis. Science. 1998; 281: 1309-1312.
43. Mahmoud M S, Fujii R, Ishikawa H, Kawano M M. Enforced CD19 expression leads to growth inhibition and reduced tumorigenicity. Blood. 1999; 94: 3551-3558.
44. Cohen J L, Boyer O, Salomon B, Onclercq R, Charlotte F, Bruel S, et al. Prevention of graft-versus-host disease in mice using a suicide gene expressed in T lymphocytes. Blood. 1997; 89: 4636-4645.
45. Spencer D M. Developments in suicide genes for preclinical and clinical applications. Curr Opin Mol Ther. 2000; 2: 433-440.
46. Lal S, Lauer U M, Niethammer D, Beck J F, Schlegel P G. Suicide genes: past, present and future perspectives. Immunol Today. 2000; 21: 48-54.
47. Kershaw M H, Teng M W, Smyth M J, Darcy P K. Supernatural T cells: genetic modification of T cells for cancer therapy. Nat Rev Immunol. 2005; 5: 928-940.
48. Chow H H, Li P, Brookshier G, Tang Y. In vivo tissue disposition of 3′-azido-3′-deoxythymidine and its anabolites in control and retrovirus-infected mice. Drug Metab Dispos. 1997; 25: 412-422.
49. Weichold F F, Jiang Y Z, Dunn D E, Bloom M, Malkovska V, Hensel N F, et al. Regulation of a graft-versus-leukemia effect by major histocompatibility complex class II molecules on leukemia cells: HLA-DR1 expression renders K562 cell tumors resistant to adoptively transferred lymphocytes in severe combined immunodeficiency mice/nonobese diabetic mice. Blood. 1997; 90: 4553-4558.

1. A composition comprising:
a stably integrating delivery vector, wherein the delivery vector comprises all or part of a nucleic acid backbone as shown in FIG. 19; and
a modified mammalian thymidylate kinase (tmpk) polynucleotide wherein the modified mammalian tmpk polynucleotide encodes a modified mammalian tmpk polypeptide that increases phosphorylation of a prodrug relative to phosphorylation of the prodrug by wild type mammalian tmpk polypeptide.

2. The composition of claim 1 wherein the tmpk polynucleotide comprises a polynucleotide with at least 80% sequence identity to a modified tmpk polynucleotide of any one of SEQ ID NOS: 15, 21, and 22.

3. The composition of claim 1 wherein the polynucleotide comprises a human tmpk polynucleotide and the polypeptide comprises a human tmpk polypeptides.

4. The composition of claim 1 wherein the modified mammalian tmpk polynucleotide comprises a mammalian tmpk polynucleotide with a point mutation.

5. The composition of claim 4 wherein the point mutation comprises a mutation in a codon of the polynucleotide selected from the group consisting of a mutation that encodes a F to Y mutation at amino acid position 105 (SEQ ID NO: 21), a mutation that encodes a R to G point mutation at amino acid position 16 (SEQ ID NO: 22), and a mutation that encodes a R to A mutation at amino acid position 200 (SEQ ID NO: 16).

6. The composition of claim 5 wherein the polynucleotide further comprises all or part of the large lid or small lid domain of E. coli (SEQ ID NO: 17).

7. The composition of claim 1 wherein the modified mammalian tmpk polynucleotide has been modified by substituting a portion of wild type tmpk polynucleotide sequence with an exogenous polynucleotide sequence.

8. The composition of claim 7 wherein the substituted portion comprises all or part of a large lid or small lid domain.

9. The composition of claim 1 further comprising a detection cassette.

10. The composition of claim 9 wherein the detection cassette is selected from the group consisting of CD19, truncated CD19, EGFP, CD25, LNGFR, truncated LNGFR, CD24, truncated CD34, EpoR, HSA and CD20.

11. The composition of claim 1 further comprising a therapeutic polynucleotide cassette selected from the group consisting of adenosine deaminase, γc interleukin receptor subunit, α-galactosidase A, acid ceramidase, galactocerebrosidase, and CFTR molecules.

12-13. (canceled)

14. A method of expressing a modified mammalian tmpk polynucleotide in a mammalian cell comprising:
contacting the mammalian cell with the composition of claim 1 wherein contacting the cell results in expression of the modified tmpk polynucleotide.

15. The method of claim 14 further comprising isolating the cells.

16. The method of claim 14 wherein the mammalian cell is a stem cell, a hematopoietic cell, a T cell and/or and a human cell.

17. The method of claim 14 wherein the mammalian cell is isolated by contacting the cell with an antibody that binds to a detection cassette protein wherein the detection cassette protein is CD19, truncated CD19, EGFP, CD25, LNGFR, truncated LNGFR, CD24, truncated CD34, EpoR, HSA and/or CD20.

18. The method of claim 14 further comprising a step wherein the isolated mammalian cell is transplanted into a mammal.

19. A method of killing a mammalian cell expressing a modified mammalian tmpk polynucleotide comprising:
contacting the mammalian cell with a composition of claim 1;
isolating the cell; and
contacting the cell with an effective amount of a prodrug to kill the cell.

20. The method of claim 19 wherein the prodrug is selected from the group consisting of thymidine analog, uracil analog, AZT, dT4 and 5-FU.

21-22. (canceled)

23. A vector construct comprising:
a stably integrating delivery vector, wherein the delivery vector comprises all or part of a nucleic acid backbone as shown in FIG. 19; and
a modified mammalian thymidylate kinase (tmpk) polynucleotide wherein the modified mammalian tmpk polynucleotide encodes a modified mammalian tmpk polypeptide that increases phosphorylation of a prodrug relative to phosphorylation of the prodrug by wild type mammalian tmpk polypeptide

24. The vector construct of claim 23 wherein the stably integrating delivery vector comprises pLTG690.

25. The vector construct of claim 23 wherein the construct comprises pLTG690 (pHR.RRE-cPPT-EF1a-Tmpk-1-CD19-WC-mPkg).

26. The vector construct of claim 23 further comprising a therapeutic nucleotide.

27. A composition comprising the vector construct of claim 23.

28. A method of expressing a modified mammalian tmpk polynucleotide in a mammalian cell comprising:
contacting the mammalian cell with the composition of claim 27,
wherein contacting the cell results in expression of the modified tmpk polynucleotide.

29. The method of claim 28 further comprising isolating the cells.

30. The method of claim 28 or 29 further transplanting the isolated mammalian cell into a mammal.

31. A method of killing a mammalian cell expressing a modified mammalian tmpk polynucleotide comprising:
contacting the mammalian cell with the composition of claim 27;
isolating the cell; and
contacting the cell with an effective amount of a prodrug to kill the cell.

32. The method of claim 31 wherein the prodrug is selected from the group consisting of thymidine analog, uracil analog, AZT, dT4 and 5-FU.

33. A method of expressing a modified mammalian tmpk polynucleotide in a subject comprising:
administering a composition of claim 1 or 27 to the subject;
wherein administration of the composition results in expression of the modified tmpk polynucleotide.

34. A method of killing a mammalian cell in a subject expressing a modified mammalian tmpk polynucleotide comprising:
administering a composition of claim 1 or 27; and
administering an effective amount of a prodrug to kill the cell.

35-38. (canceled)

39. An isolated cell that expresses a modified tmpk according to the method of claim 14 or 28.

40. The isolated cell of claim 39 wherein the cell is a cell from a transplant patient.

41. A method of treating GVHD comprising transplanting a cell according to claim 39.

42. A method of treating cancer comprising transplanting a cell according to claim 39.

43. The method of claim 41 or 42 further comprising administering a prodrug that kills the transplanted cell.


SEQUENCE LISTING (31 sequences)

SEQ ID NO: 1 - DNA, 639 bp, Homo sapiens
atggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcaccggtgccaaggagaatttttccctagattggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatccgcactgccacagagaagccgctgggggagctatggaagtga

SEQ ID NO: 2 - protein, 212 aa, Homo sapiens
MAARRGALIVLEGVDRAGKSTQSRKLVEALCAAGHRAELLRFPERSTEIGKLLSSYLQKKSDVEDHSVHLLFSANRWEQVPLIKEKLSQGVTLVVDRYAFSGVAFTGAKENFSLDWCKQPDVGLPKPDLVLFLQLQLADAAKRGAFGHERYENGAFQERALRCFHQLMKDTTLNWKMVDASKSIEAVHEDIRVLSEDAIRTATEKPLGELWK

SEQ ID NO: 3 - DNA, 639 bp, Homo sapiens; identical to SEQ ID NO: 1

SEQ ID NO: 4 - protein, 212 aa, Homo sapiens; identical to SEQ ID NO: 2

SEQ ID NO: 5 - DNA, 636 bp, Homo sapiens
atggcggcccggcgcggggctctcatagtgctggagggcgtggaccgcgccgggaagagcacgcagagccgcaagctggtggaagcgctgtcgcgcgggccaccgcccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcaccggtgccaaggagaatttttccctagactggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagactcgaagctgtccatgaggaactccgcgtgctctctgaggacgccatccgcactgccacagagaagccgctgggggagctatggaagtga

SEQ ID NO: 6 - protein, 211 aa, Homo sapiens
MAARRGALIVLEGVDRAGKSTQSRKLVEALSRGPPPELLRFPERSTEIGKLLSSYLQKKSDVEDHSVHLLFSANRWEQVPLIKEKLSQGVTLVVDRYAFSGVAFTGAKENFSLDWCKQPDVGLPKPDLVLFLQLQLADAAKRGAFGHERYENGAFQERALRCFHQLMKDTTLNWKMVDASKRLEAVHEELRVLSEDAIRTATEKPLGELWK

SEQ ID NO: 7 - DNA, 639 bp, Homo sapiens
atggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcaccggtgccaaggagaatttttccctagattggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatccgcactgccacagagaagccgctgggggagctatggaaggac

SEQ ID NO: 8 - protein, 213 aa, Homo sapiens
MAARRGALIVLEGVDRAGKSTQSRKLVEALCAAGHRAELLRFPERSTEIGKLLSSYLQKKSDVEDHSVHLLFSANRWEQVPLIKEKLSQGVTLVVDRYAFSGVAFTGAKENFSLDWCKQPDVGLPKPDLVLFLQLQLADAAKRGAFGHERYENGAFQERALRCFHQLMKDTTLNWKMVDASKSIEAVHEDIRVLSEDAIRTATEKPLGELWKD

SEQ ID NO: 9 - DNA, 639 bp, Mus musculus
atggcgtcgcgtcggggagcgctcatcgtgctggagggtgtggaccgtgctggcaagaccacgcagggcctcaagctggtgaccgcgctgtgcgcctcgggccacagagcggagctgctgcgtttccccgaaagatcaacggaaatcggcaagcttctgaattcctacttggaaaagaaaacggaactagaggatcactccgtgcacctgctcttctctgcaaaccgctgggaacaagtaccattaattaaggcgaagttgaaccagggtgtgacccttgttttggacagatacgccttttctggggttgccttcactggtgccaaagagaatttttccctggattggtgtaaacaaccggacgtgggccttcccaaacctgacctgatcctgttccttcagttacaattgctggacgctgctgcacggggagagtttggccttgagcgatatgagaccgggactttccaaaagcaggttctgttgtgtttccagcagctcatggaagagaaaaacctcaactggaaggtggttgatgcttccaaaagcattgaggaagtccataaagaaatccgtgcacactctgaggacgccatccgaaacgctgcacagaggccactgggggagctatggaaataa

SEQ ID NO: 10 - protein, 212 aa, Mus musculus
MASRRGALIVLEGVDRAGKTTQGLKLVTALCASGHRAELLRFPERSTEIGKLLNSYLEKKTELEDHSVHLLFSANRWEQVPLIKAKLNQGVTLVLDRYAFSGVAFTGAKENFSLDWCKQPDVGLPKPDLILFLQLQLLDAAARGEFGLERYETGTFQKQVLLCFQQLMEEKNLNWKVVDASKSIEEVHKEIRAHSEDAIRNAAQRPLGELWK

SEQ ID NO: 11 - protein, 212 aa, Homo sapiens
MAARRGALIVLEGVDRAGKSTQSRKLVEALCAAGHRAELLRFPERSTEIGKLLSSYLQKKSDVEDHSVHLLFSANRWEQVPLIKEKLSQGVTLVVDRYAFSGVAYTGAKENFSLDWCKQPDVGLPKPDLVLFLQLQLADAAKRGAFGHERYENGAFQERALRCFHQLMKDTTLNWKMVDASKSIEAVHEDIRVLSEDAIRTATEKPLGELWK

SEQ ID NO: 12 - protein, 214 aa, Homo sapiens
MAARRGALIVLEGVDGAGKSTQSRKLVEALCAAGHRAELLRFPERSTEIGKLLSSYLQKKSDVEDHSVHLLFSANRWEQVPLIKEKLSQGVTLVVDRYAFSGVAFTGAKENFSLDWCKQPDVGLPKPDLVLFLQLTPEVGLKRARARGQLDRYENGAFQERALRCFHQLMKDTTLNWKMVDASKSIEAVHEDIRVLSEDAIATATEKPLGELWK

SEQ ID NO: 13 - DNA, 6811 bp, Artificial Sequence
  lentiviral delivery vector
tggaagggctaattcactcccaacgaagacaagatatccttgatctgtggatctaccacacacaaggctacttccctgattggcagaactacacaccaggaccagggatcagatatccactgacctttggatggtgctacaagctagtaccagttgagccagataaggtagaagaggccaacaaaggagagaacaccagcttgttacaccctgtgagcctgcatggaatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcttttaaaagaaaaggggggattggggggtacagtgcaggggaaagaatagtagacataatagcaacagacatacaaactaaagaattacaaaaacaaattacaaaaattcaaaattttatcgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagaggaattctgcagtcgagcggagcgcgcgtaatacgactcactatagggcgccatgggtaccgggccccccctcgatcgaacaacaacaacaataacacatggttccgcgtggctctcatatggcggcccggcgcggggctctcatagtgctggagggcgtggacggcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcaccggtgccaaggagaatttttccctagactggtgtaaacagccagacgtgggccttcccaaacccgacctggtcctgttcctgcagttaactccggaagttggcttaaaacgcgcacgtgctcgcggcgagcttgaccgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatcgccactgccacagagaagccgctgggggagctatggaagtgaggatcagtcgacggtatcgattccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgatatcgaattcctgcagcccgggggatccgccccctctgaccaccatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggcatcctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatctttaaggcgcgccccgggatccaagcttcaattgtggtcactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 14 - DNA, 6805 bp, Artificial Sequence
  lentiviral delivery vector
tggaagggctaattcactcccaacgaagacaagatatccttgatctgtggatctaccacacacaaggctacttccctgattggcagaactacacaccaggaccagggatcagatatccactgacctttggatggtgctacaagctagtaccagttgagccagataaggtagaagaggccaacaaaggagagaacaccagcttgttacaccctgtgagcctgcatggaatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcttttaaaagaaaaggggggattggggggtacagtgcaggggaaagaatagtagacataatagcaacagacatacaaactaaagaattacaaaaacaaattacaaaaattcaaaattttatcgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagaggaattctgcagtcgagcggagcgcgcgtaatacgactcactatagggcgccatgggtaccgggccccccctcgatcgaacaacaacaacaataacacatggttccgcgtggctctcatatggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggcctacacaggtgccaaggagaatttttccctagactggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatcgccactgccacagagaagccgctgggggagctatggaagtgaggatcagtcgacggtatcgattccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgatatcgaattcctgcagcccgggggatccgccccctctgaccaccatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggcatcctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatctttaaggcgcgccccgggatccaagcttcaattgtggtcactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 15 - DNA, 638 bp, Homo sapiens
tggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcacaggtgccaaggagaatttttccctagactggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatccgcactgccacagagaagccgctgggggagctatggaagtga

SEQ ID NO: 16 - protein, 211 aa, Homo sapiens
MAARRGALIVLEGVDRAGKSTQSRKLVEALCAAGHRAELLRFPERSTEIGKLLSSYQKKSDVEDHSVHLLFSANRWEQVPLIKEKLSQGVTLVVDRYAFSGVAFTGAKENFSLDWCKQPDVGLPKPDLVLFLQLQLADAAKRGAFGHERYENGAFQERALRCFHQLMKDTTLNWKMVDASKSIEAVHEDIRVLSEDAIATATEKPLGELWK

SEQ ID NO: 17 - protein, 15 aa, Escherichia coli
TPEVGLKRARARGEL

SEQ ID NO: 18 - DNA, 118 bp, Human immunodeficiency virus
ttttaaaagaaaaggggggattggggggtacagtgcaggggaaagaatagtagacataatagcaacagacatacaaactaaagaattacaaaaacaaattacaaaaattcaaaatttt

SEQ ID NO: 19 - DNA, 592 bp, Woodchuck hepatitis B virus
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 20 - protein, 13 aa, Homo sapiens
QLADAAKRGAFGH

SEQ ID NO: 21 - DNA, 639 bp, Homo sapiens
atggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggcctacacaggtgccaaggagaatttttccctagactggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatcgccactgccacagagaagccgctgggggagctatggaagtga

SEQ ID NO: 22 - DNA, 645 bp, Homo sapiens
atggcggcccggcgcggggctctcatagtgctggagggcgtggacggcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcaccggtgccaaggagaatttttccctagactggtgtaaacagccagacgtgggccttcccaaacccgacctggtcctgttcctgcagttaactccggaagttggcttaaaacgcgcacgtgctcgcggcgagcttgaccgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatcgccactgccacagagaagccgctgggggagctatggaagtga

SEQ ID NO: 23 - DNA, 28 bp, Homo sapiens
atgccacctcctcgcctcctcttcttcc

SEQ ID NO: 24 - DNA, 22 bp, Homo sapiens
tcacctggtgctccaggtgccc

SEQ ID NO: 25 - DNA, 23 bp, Homo sapiens
ccgccaccgcggtggagctccag

SEQ ID NO: 26 - DNA, 26 bp, Homo sapiens
ttaaagatgaagaatgcccacaaggg

SEQ ID NO: 27 - DNA, 1966 bp, Homo sapiens
aggcccctgcctgccccagcatcccctgcgcgaagctgggtgccccggagagtctgaccaccatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggccccagaaccagtacgggaacgtgctgtctctccccacacccacctcaggcctcggacgcgcccagcgttgggccgcaggcctggggggcactgccccgtcttatggaaacccgagcagcgacgtccaggcggatggagccttggggtcccggagcccgccgggagtgggcccagaagaagaggaaggggagggctatgaggaacctgacagtgaggaggactccgagttctatgagaacgactccaaccttgggcaggaccagctctcccaggatggcagcggctacgagaaccctgaggatgagcccctgggtcctgaggatgaagactccttctccaacgctgagtcttatgagaacgaggatgaagagctgacccagccggtcgccaggacaatggacttcctgagccctcatgggtcagcctgggaccccagccgggaagcaacctccctggggtcccagtcctatgaggatatgagaggaatcctgtatgcagccccccagctccgctccattcggggccagcctggacccaatcatgaggaagatgcagactcttatgagaacatggataatcccgatgggccagacccagcctggggaggagggggccgcatgggcacctggagcaccaggtgatcctcaggtggccagcctggatctcctcaagtccccaagattcacacctgactctgaaatctgaagacctcgagcagatgatgccaacctctggagcaatgttgcttaggatgtgtgcatgtgtgtaagtgtgtgtgtgtgtgtgtgtgtgtatacatgccagtgacacttccagtcccctttgtattccttaaataaactcaatgagctcttccaaaaaaaaaaaaaaaaaa

SEQ ID NO: 28 - protein, 556 aa, Homo sapiens [sequence omitted]

SEQ ID NO: 29 - protein, 313 aa, Homo sapiens
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHL

SEQ ID NO: 30 - DNA, 939 bp, Homo sapiens
  exon (1)..(939)
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatctt

SEQ ID NO: 31 - protein, 313 aa, Homo sapiens
MPPPRLLFFLLFLTPMEVRPEEPLVVKVEEGDNAVLQCLKGTSDGPTQQLTWSRESPLKPFLKLSLGLPGLGIHMRPLASWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHL